(19) United States
(12) Patent Application Publication
(10) Pub. No.: US 2025/0263750 A1
(43) Pub. Date: Aug. 21, 2025

(54) DETERMINISTIC MECHANOPORATION DEVICES, SYSTEMS AND METHODS

(71) Applicant: Basilard BioTech, Inc., Temecula, CA (US)

(72) Inventors: James ONUFFER, Alameda, CA (US); Faysal AHMED, San Diego, CA (US); Aaron CHRISTENSEN-QUICK, San Diego, CA (US); Brynley LEE, Temecula, CA (US); Masaru P. RAO, Riverside, CA (US); Dennis YOST, Los Gatos, CA (US)

(21) Appl. No.: 18/935,212

(22) Filed: Nov. 1, 2024

Related U.S. Application Data
(63) Continuation of application No. PCT/US2023/020744, filed on May 2, 2023.
(60) Provisional application No. 63/337,928, filed on May 3, 2022.
(60) Provisional application No. 63/380,483, filed on Oct. 21, 2022.

(51) Int. Cl. C12N 15/87

(52) U.S. Cl. CPC C12N 15/87

(57) ABSTRACT

Described are systems, devices, and methods for delivery of a payload to a cell, including gene transfection of the cell. Such systems, devices, and methods are configured to capture and form a pore in a cell through which the payload may be delivered. Additionally, such systems, devices and methods are configured to work with various cell types and to deliver the payloads to the nucleus of the cell.

### CROSS-REFERENCE TO RELATED APPLICATIONS

This application is a continuation of International Application PCT/US2023/020744, filed on May 2, 2023, which claims the benefit of U.S. Provisional Application No. 63/337,928, filed on May 3, 2022, and U.S. Provisional Application No. 63/380,483, filed Oct. 21, 2022, all of which are hereby incorporated by reference in their entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Intracellular delivery of exogenous materials into large populations of suspension cells is required for a growing number of life science applications. Therefore, there is a need for devices, systems and methods allowing for high throughput and efficient intracellular delivery of exogenous materials while maintaining viability of the cells after delivery. Gene transfection is one such application.

### SUMMARY

Provided herein are methods and systems for precision mechanoporation. In some embodiments, mechanoporation may comprise capturing a cell, mechanically creating a pore in the cell, and injecting a cargo into the cell or a section thereof through the pore. In some examples, a pore may be generated in the cells to inject, deliver, or facilitate a delivery of a cargo into the nucleus of the cell. In some examples, a cargo may be injected/delivered into the cell to transfect the cell.

In an aspect, provided herein is a system for precision single-cell mechanoporation comprising: (a) a static array of wells, wherein each well of the array of wells is configured to capture a single cell therein. In some cases, cell capture may be performed via sedimentation (e.g., gravitational sedimentation) and flow-focusing of the single cell; (b) a static needle in the bottom surface of each well of the static array of wells configured to inject a cargo into the nucleus of the single cell captured in the well, in some cases without back-and-forth movement of the needle; and (c) one or more modules for generating a pressure gradient, wherein the pressure gradient is configured to induce a pressure-driven flow of a single cell suspension in the system, thereby leading to gravitational sedimentation, flow-focusing, and capture of the single cell in the well. In some embodiments, capturing the single cell in the well is accomplished without centrifugation of the system or the static array.

In some embodiments, the length of the needle to the depth of the well comprises a ratio of about 30% to about 60%. In some embodiments, the length of the needle to the depth of the well comprises a ratio of about 45% to about 65%. In some embodiments, the length of the needle to the depth of the well comprises a ratio of approximately 50%. In some embodiments, the needle (e.g., the static needle) has a length of from 1 micron to 8 microns, from 2 microns to 4 microns, and/or from 3 microns to 4 microns.

In some embodiments, the well comprises a diameter of at most about 20 micrometers (microns), 18 microns, 16 microns, 14 microns, 12 microns, 10 microns, 8 microns, or smaller.

In some embodiments, the system is capable of processing at least 0.5 million cells per cycle. In some embodiments, the system is capable of processing at least 1 million cells per cycle. In some embodiments, the system further comprises a computer software for controlling the performance of the system.

In an aspect, provided herein is a method of cell mechanoporation which may in some cases be used for intracellular cargo delivery, the method comprising: (a) providing or obtaining a mechanoporation system, wherein the mechanoporation system comprises: (i) a static array of wells, wherein each well of the static array of wells is configured to capture a single cell therein via sedimentation (e.g., gravitational sedimentation) and flow-focusing of the single cell; (ii) a static needle in the bottom surface of each well of the static array of wells; (iii) one or more modules for generating a pressure gradient in the system; and, (iv) one or more liquid reservoirs.

The method further comprises (b) providing or obtaining a cell suspension in the one or more liquid reservoirs of (iv); (c) generating a pressure gradient across the one or more liquid reservoirs of (iv) and the static array of wells (i) using the one or more modules of (iii), thereby generating a pressure-driven flow (e.g., pressure-driven microfluidic flow) of the cell suspension from the reservoirs across the static array of wells; (d) capturing single cells from the cell suspension in the wells of the static array of wells, without centrifugation, thereby generating a single captured cell in the well; and, (e) creating a pore in the single captured cell, and injecting/delivering a cargo into the single cell or a section or component thereof (e.g., nucleus of the single captured cell) using the static needle of (ii), in some cases, without back-and-forth movement of the needle.

In some embodiments, the single captured cell remains viable after performing the method thereon. In some embodiments, at least about 70% of a population of cells processed using the method remain viable. In some embodiments, at least about 90% of a population of cells processed using the method remain viable. In some embodiments, at least about 10% of the wells of the static array of wells capture a single cell therein upon performing the method. In some embodiments, at least about 15% of the wells of the static array of wells capture a single cell therein upon performing the method. In some embodiments, at least about 20% of the wells of the static array of wells capture a single cell therein upon performing the method. In some embodiments, at least about 40% of the wells of the static array of wells capture a single cell therein upon performing the method.

In some embodiments, the cargo load delivered into individual cells among a population of cells processed using the method, is substantially uniform. In some embodiments, a coefficient of variability (% CV) in cargo load across individual cells among a population of cells processed using the method is at most about 50%. In some embodiments, a coefficient of variability (% CV) in cargo load across individual cells among a population of cells processed using the method is at most about 40%. In some embodiments, a coefficient of variability (% CV) in cargo load across individual cells among a population of cells processed using the method is at most about 30%. In some embodiments, a coefficient of variability (% CV) in cargo load across individual cells among a population of cells processed using the method is at most about 20%. In some embodiments, a coefficient of variability (% CV) across individual cells among a population of cells processed using the method is at most about 10%. In some embodiments, a coefficient of variability (% CV) across individual cells among a population of cells processed using the method is at most about 5%. In some embodiments, the needle (e.g., the static needle) has a length of from 1 micron to 8 microns, from 2 microns to 4 microns, and/or from 3 microns to 4 microns.

In some embodiments, the system comprises: a substrate comprising: a surface; and a capture site, the capture site comprising: a well or trap having a well boundary shape at the surface and having a well depth, the well configured to support a cell; and a projection/needle extending from the bottom of the well toward the surface of the substrate, the projection/needle comprising a projection/needle length, and wherein the projection/needle length and the well depth comprise a projection length-well depth ratio. In some embodiments, the projection/needle length-well depth ratio is about 25% to about 100%. In some embodiments, the projection length-well depth ratio is about 40% to about 60%. In some embodiments, the projection length-well depth ratio is about 50%. In some embodiments, the well boundary shape comprises a circle, ellipse, square, rectangle, triangle, or any combination thereof. In some embodiments, the well boundary shape comprises a boundary shape area. In some embodiments, the well boundary shape comprises a well diameter. In some embodiments, the well diameter is about 10 microns to about 15 microns. In some embodiments, the well diameter is about 10 microns. In some embodiments, the port comprises a port boundary shape. In some embodiments, the port boundary shape comprises a circle, ellipse, square, rectangle, triangle, arc, or combination thereof. In some embodiments, the port boundary shape comprises a long axis. In some embodiments, the port comprises a plurality of ports. In some embodiments, the port comprises an aspiration via. In some embodiments, the port comprises a lumen. In some embodiments, the projection/needle comprises the lumen. In some embodiments, the plurality of ports comprises a lumen and an aspiration via. In some embodiments, the port area and boundary shape area comprise a port area-boundary shape area ratio. In some embodiments, the needle/projection length is about 3 to about 4 microns. In some embodiments, the needle/projection length is about 3.5 microns. In some embodiments, the well depth is about 4 microns to about 6 microns. In some embodiments, the well depth is about 5 microns. In some embodiments, the handle layer thickness is about 600 microns to about 700 microns. In some embodiments, the handle layer thickness is at least about 650 microns. In some embodiments, the handle layer thickness is about 675 microns. In some embodiments, the device layer thickness is about 25 microns to about 35 microns. In some embodiments, the device layer thickness is at least about 25 microns. In some embodiments, the device layer thickness is about 30 microns.

A system for gene transfection, the system comprising: a device in accordance with some embodiments; and a pressure source; wherein, the pressure source is configured to produce a pressure time-waveform allowing for the capture of, perforation of, payload insertion to and release of a cell.

A method for gene transfection comprising: obtaining the system or the device in accordance with some embodiments; introducing the cell to the surface; applying a pressure-time waveform and allowing for capture of, perforation of and release of the cell; and allowing for insertion of a payload into the cell.

A method for gene transfection comprising: obtaining a system or device in accordance with some embodiments; introducing a cell to the surface; applying a pressure-time waveform and allowing for capture, perforation, insertion of a payload into the cell and release of the cell. In some embodiments, the pressure-time waveforms are applied across the port area. In some embodiments, the port comprises at least one port. In some embodiments, the pressure-time waveform comprises, a square shaped, a pulse-shaped, a sinusoidal-shaped, a chirp-shaped or any shape of waveform. In some embodiments, wherein the pressure-time waveform comprises a maximum pressure of about 300 mbar to about 500 mbar. In some embodiments, the pressure-time waveform comprises a duration of about 10 minutes to about 20 minutes. In some embodiments, the pressure-time waveform comprises at least one cycle. In some embodiments, the cycle comprises a pulse. In some embodiments, the pressure-time waveform comprises the pulse. In some embodiments, the pressure time-waveform comprises a plurality of pulses. In some embodiments, the pressure time waveform comprising the plurality of pulses is referred to as a pulsatile waveform. In some embodiments, the pulsatile waveform is configured to produce pulsatile flow. In some embodiments, the pulsatile flow results in capture and release of the cell. In some embodiments, the pulsatile flow results in: capturing; compressing; perforating; recoiling; inserting of payload; and releasing of the cell, and wherein the capturing, compressing, perforating, recoiling, inserting of payload and releasing of the cell may or may not occur at the same time, and may occur in any order.

Described herein are various embodiments of a system, comprising: a substrate comprising: a surface; and a capture site, the capture site comprising: a well having a well boundary shape at the surface and having a well depth, the well configured to support a cell; and a projection/needle extending from the bottom of the well toward the surface of the substrate, the projection comprising a projection/needle length, and wherein the projection/needle length and the well depth comprise a projection length-well depth ratio. In some embodiments, the projection/needle length-well depth ratio is about 25% to about 100%. In some embodiments, the projection/needle length-well depth ratio is about 40% to about 60%. In some embodiments, the projection/needle length-well depth ratio is about 50%. In some embodiments, the well boundary shape comprises a clover leaf, a circle, an ellipse, a square, a rectangle, or a triangle. In some embodiments, the well boundary shape comprises a well boundary shape area. In some embodiments, the well boundary shape comprises a well diameter. In some embodiments, the well diameter is about 2 microns to about 30 microns. In some embodiments, the well diameter is about 5 microns to about 15 microns. In some embodiments, the well diameter is about 10 microns to about 12 microns. In some embodiments, the well diameter is about 11.5 microns to about 12.5 microns. In some embodiments, the well diameter is about 12 microns. In some embodiments, the port comprises a port boundary shape. In some embodiments, the port boundary shape is an arc, a circle, an ellipse, a square, a rectangle, or triangle. In some embodiments, the port boundary shape comprises a long axis. In some embodiments, the port boundary long axis is about 1 micron to about 10 microns. In some embodiments, the port boundary long axis is about 2 microns to about 6 microns. In some embodiments, the port boundary long axis is about 4 microns. In some embodiments, the port boundary shape comprises a short axis. In some embodiments, the port boundary shape short axis is about 0.1 microns to about 8 microns. In some embodiments, the port boundary shape short axis is about 0.2 microns to about 4 microns. In some embodiments, the port boundary long axis is about 2 microns. In some embodiments, the port comprises a plurality of ports. In some embodiments, the port comprises an aspiration via. In some embodiments, the port comprises a lumen. In some embodiments, the projection/needle comprises the lumen. In some embodiments, the plurality of ports comprises a lumen and an aspiration via. In some embodiments, a port area and well boundary shape area comprise a port area-well boundary shape area ratio. In some embodiments, the port area-well boundary shape area ratio is about 1% to about 50%. In some embodiments, the port area-well boundary shape area ratio is about 2% to about 10%. In some embodiments, the port area-well boundary shape area ratio is about 5% to about 8%. In some embodiments, the port area is 6.28 microns squared, and a well boundary shape area is 113 microns squared. In some embodiments, the port area is 6.28 microns squared, and the well boundary shape area is 78.5 microns squared. In some embodiments, a port-to-port gap is about 0.1 microns to about 10 microns. In some embodiments, the port-to-port gap is about 0.5 microns to about 5 microns. In some embodiments, the port-to-port gap is about 1 micron. In some embodiments, the aspiration via comprising an aspiration via channel length of about 1 micron to about 100 microns. In some embodiments, the port is an aspiration via comprising an aspiration via channel length of about 5 microns to about 50 microns. In some embodiments, the port is an aspiration via comprising an aspiration via channel length of about 24 microns to about 25 microns. In some embodiments, the aspiration via channel length of about 23.5 microns to about 24.5 microns. In some embodiments, the port is an aspiration via comprising an aspiration via channel length is about 24 microns. In some embodiments, the device further comprises a device (e.g., chip) shape, the device shape comprising a square, circle, ellipse, rectangle, or a triangle. In some embodiments, the device shape is a square and comprising a device edge length. In some embodiments, the device edge length may be about 5 millimeters to about 100 millimeters. In some embodiments, the device edge length may be about 21 millimeters to about 25 millimeters. In some embodiments, the device further comprises a sub-array. In some embodiments, a gap between the sub-array and a neighboring sub-array is about 10 microns to about 4000 microns. In some embodiments, the gap between the sub-array and the neighboring sub-array is about 100 microns to about 400 microns. In some embodiments, the gap between the sub-array and the neighboring sub-array is about 200 microns. In some embodiments, the sub-array shape comprises a square, circle, ellipse, rectangle, or triangle shape. In some embodiments, the sub-array is a square. In some embodiments, the square sub-array comprises an edge. In some embodiments, the square sub-array comprises an edge length. In some embodiments, the square sub-array edge length is about 10 microns to about 20 millimeters. In some embodiments, the square sub-array edge length is about 1000 microns to about 2000 microns. In some embodiments, the square sub-array edge length is about 1373 microns to about 1383 microns. In some embodiments, the edge length is about 1373 microns. In some embodiments, an array of arrays (e.g., sub-array layout) comprises 1 row of sub-arrays to 100 rows of sub-arrays, and the array of arrays (e.g., sub-array layout) comprises 1 column of sub-arrays to 100 columns of sub-arrays. In some embodiments, the array of arrays (e.g., sub-array layout) comprises 5 rows of sub-arrays to 50 rows of sub-arrays, and the array of arrays (e.g., sub-array layout) comprises 5 columns of sub-arrays to 50 columns of sub-arrays. In some embodiments, the array of arrays (e.g., sub-array layout) comprises 10 rows of sub-arrays and 10 columns of sub-arrays. In some embodiments, the array of arrays (e.g., sub-array layout) comprises a critical feature area. In some embodiments, the critical feature area comprises a length and a width. In some embodiments, the critical feature area length is about 1 millimeter to about 50 millimeters. In some embodiments, the critical feature area length is about 10 millimeters to about 20 millimeters. In some embodiments, the critical feature area length is about 13.6 millimeter to about 15.5 millimeters. In some embodiments, the critical feature area width is about 1 millimeter to about 50 millimeters. In some embodiments, the critical feature area width is about 10 millimeters to about 20 millimeters. In some embodiments, the critical feature area width is about 13.6 millimeter to about 15.5 millimeters.

The devices and systems disclosed herein may comprise a needle, a projection, or a needle-like project in the well. This may be also referred to as a static needle. The terms needle, projection, needle-like projection, and static needle may be used interchangeably throughout the disclosure.

In some embodiments, the projection/needle length is about 1 micron to about 8 microns. In some embodiments, the projection/needle length is about 3 microns to about 4 microns. In some embodiments, the projection/needle length is about 3.5 microns. In some embodiments, a projection/needle length recess from the surface is about 0 microns to about 7 microns. In some embodiments, a projection/needle length recess from the surface is about 2 microns to about 3 microns. In some embodiments, a projection/needle length recess from the surface is about 1.5 microns to about 2.5 microns. In some embodiments, a projection/needle length recess from the surface is about 2.5 microns. In some embodiments, the projection/needle comprises a projection/needle base diameter of about 1 micron. In some embodiments, the projection/needle comprises a projection/needle tip diameter. In some embodiments, the projection/needle tip diameter is about 50 nanometers to about 200 nanometers. In some embodiments, a projection/needle tip diameter-to-projection/needle base diameter ratio is about 1% to about 50%. In some embodiments, the projection/needle tip diameter-to-projection base diameter ratio is about 5% to about 25%. In some embodiments, the projection/needle tip diameter-to-projection/needle base diameter ratio is about 10%. In some embodiments, the well depth is about 2 microns to about 12 microns. In some embodiments, the well depth is about 4 microns to about 6 microns. In some embodiments, the well depth is about 5 microns to about 6 microns. In some embodiments, the well depth is about 5.5 microns to about 6.5 microns. In some embodiments, the well depth is about 6 microns. In some embodiments, the well boundary shape area is about 3 square microns to about 200 square microns. In some embodiments, the well boundary shape area is about 75 square microns to about 115 square microns. In some embodiments, the well boundary shape area is about 70 square microns to about 175 square microns. In some embodiments, the well comprises a well center-to-center spacing (e.g., well diagonal and lateral spacing). In some embodiments, the well center-to-center spacing is about 5 microns to about 30 microns. In some embodiments, the well center-to-center spacing is about 7 microns to about 21 microns. In some embodiments, the well center-to-center spacing (e.g., well diagonal and lateral spacing) is about 12 microns to about 14 microns. In some embodiments, the well center-to-center spacing (e.g., well diagonal and lateral spacing) is about 13.5 microns to about 14.5 microns. In some embodiments, the well center-to-center spacing (e.g., well diagonal and lateral spacing) is about 14 microns. In some embodiments, the sub-array comprises a sub-array layout pattern. In some embodiments, the sub-array layout pattern comprises a hexagonal pattern (e.g., honeycomb or diamond pattern), or a linear (e.g., square) pattern. In some embodiments, the sub-array comprises a well-to-well gap spacing. In some embodiments, the well-to-well gap spacing is about 1 micron to about 50 microns. In some embodiments, the well-to-well gap spacing is about 1.5 to about 2.5 microns. In some embodiments, the well-to-well gap spacing is about 2 microns. In some embodiments, the device further comprises a handle layer thickness. In some embodiments, the handle layer thickness is at least about 525 microns. In some embodiments, the handle layer thickness is about 525 microns to about 750 microns. In some embodiments, the handle layer thickness is about 590 microns to about 650 microns. In some embodiments, the handle layer thickness is about 610 microns. In some embodiments, the device further comprises a device layer thickness. In some embodiments, the device layer thickness is about 25 microns to about 35 microns. In some embodiments, the device layer thickness is at least about 30 microns. In some embodiments, the device layer thickness is about 30 microns. In some embodiments, the device further comprises a buried oxide layer. In some embodiments, the buried oxide layer comprises a buried oxide layer thickness. In some embodiments, the buried oxide layer thickness is about 0.1 microns to about 100 microns. In some embodiments, the buried oxide layer thickness is about 1 micron to about 10 microns. In some embodiments, the buried oxide layer thickness is about 2 microns. In some embodiments, the device further comprises a dopant. In some embodiments, the dopant is a p-type dopant. In some embodiments, the p-type dopant is boron. In some embodiments, the dopant is an n-type dopant. In some embodiments, the n-type dopant is phosphorous, arsenic or antimony.

Described herein are various embodiments of a system for gene transfection, the system comprising: a device in accordance with some embodiments; and a pressure source; wherein, the pressure source is configured to produce a pressure time-waveform allowing for the capture of, perforation of, payload insertion to and release of a cell. In some embodiments, the system further comprises a cartridge assembly, a vacuum source, a pressure controller, a vacuum controller, a fluid reservoir, a flow sensor, a pressure sensor, a valve, a valve controller, or a combination thereof.

Described herein are various embodiments of a method for gene transfection, comprising: obtaining the system, or the device, in accordance with some embodiments; introducing the cell to the surface; applying a pressure-time waveform and allowing for capture of, perforation of and release of the cell; and allowing for insertion of a payload into the cell.

Described herein are various embodiments of a method for gene transfection, comprising: obtaining the system or the device, in accordance with some embodiments; introducing a cell to the surface; applying a pressure-time waveform and allowing for capture, perforation, insertion of a payload into the cell and release of the cell. In some embodiments, the pressure-time waveforms are applied across the port area. In some embodiments, the port comprises at least one port. In some embodiments, the pressure-time waveform comprises, a square shaped waveform, a pulse-shaped waveform, a sinusoidal-shaped waveform, a chirp-shaped waveform, or any shape of waveform. In some embodiments, the pressure-time waveform comprises a maximum pressure of about 100 mbar to about 6000 mbar. In some embodiments, the pressure-time waveform comprises a maximum pressure of about 300 mbar to about 500 mbar. In some embodiments, the pressure-time waveform comprises a duration of less than about 21 minutes. In some embodiments, the pressure-time waveform comprises a duration of about 20 minutes or less. In some embodiments, the pressure-time waveform comprises a duration of about 10 minutes to about 20 minutes. In some embodiments, the pressure-time waveform comprises at least one cycle. In some embodiments, the at least one cycle comprises a pulse. In some embodiments, the pressure-time waveform comprises the pulse. In some embodiments, the pressure time-waveform comprises a plurality of pulses. In some embodiments, the pressure time waveform comprising the plurality of pulses is referred to as a pulsatile waveform. In some embodiments, the pulsatile waveform is configured to produce a pulsatile flow. In some embodiments, the pulsatile flow results in capture and release of the cell. In some embodiments, the pulsatile flow results in: capturing; compressing; perforating; recoiling; inserting of payload; and releasing of the cell, and wherein the capturing, compressing, perforating, recoiling, inserting of payload and releasing of the cell may or may not occur at the same time, and may occur in any order.

The system, in accordance with some embodiments, may be configured to perform the methods as described herein in accordance with some embodiments.

Various embodiments of the method as described herein may be performed on any industrial system.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-IF show non-limiting examples of a gene delivery device.

FIG. 2 shows a non-limiting example of a gene dene delivery device.

FIGS. 3A-3C show non-limiting examples of a gene dene delivery device.

FIGS. 4A-4M show results from a simulation involving design stress analysis.

FIGS. 5A-5E show non-limiting examples of port shapes.

FIGS. 6A-6E show non-limiting examples of various port (e.g., aspiration via) arrangements.

FIG. 7 shows non-limiting examples of a full chip array, sub-array and a single capture site.

FIG. 8 shows results for max fluid pressure testing (e.g., release flow across a chip).

FIGS. 9A-9C show schematics of a constant pressure system and a cartridge assembly, according to some embodiments.

FIGS. 10A-10C show schematics of various capture site designs, according to some embodiments.

FIGS. 11A-11C show non-limiting examples of capture sites, including port (e.g., aspiration via) design and area statistics.

FIGS. 12A-12B show non-limiting examples of alternative Next Gen Prototype V2—Arc Via designs.

FIGS. 13A-13B show non-limiting examples of capture sites, including port (e.g., aspiration via) design and area statistics.

FIGS. 14A-14B show non-limiting examples of aspiration via designs comprising overlapping arc vias of different dimensions.

FIGS. 15A-15B show non-limiting examples of circular port (e.g., aspiration via) designs with dimensions.

FIGS. 16A-16B show non-limiting examples of capture site patterning and spacing.

FIGS. 17A-17C show non-limiting example projection/needle heights/lengths for various designs in accordance with some embodiments.

FIGS. 18A-18B show results of a flow velocity magnitude simulation.

FIGS. 19A-19B show results of a flow velocity magnitude simulation.

FIGS. 20A-20B show results of a flow velocity magnitude simulation.

FIGS. 21A-21B show a cross section and simulation data for a Next Gen Prototype V2—Arc Via design with different projection (e.g., needle/static needle/needle-like projection) heights/lengths.

FIGS. 22A-22C show various views of a cartridge assembly in accordance with some embodiments.

FIGS. 23A-23C show various views of a chip card in accordance with some embodiments.

FIGS. 24A-24B show examples of chip card assemblies in accordance with some embodiments.

FIG. 25 shows a non-limiting example of a system unit operation procedure in accordance with some embodiments.

FIG. 26 shows a non-limiting example of an assembly of chips in accordance with some embodiments.

FIGS. 27A-27C show results of a simulation of principal stresses of the device in accordance with some embodiments.

FIGS. 28A-28B show results of a design simulation showing that selective capture site removal in highest stress regions reduces maximum principal stress.

FIGS. 29A-29C show non-limiting example images of selective site removal implemented in fabricated device chips.

FIGS. 30A-30B show results of design simulations (at full device level) demonstrating that increasing device and handle layer thicknesses reduce max principal stresses.

FIGS. 31A-31C show various examples of device designs in accordance with some embodiments.

FIG. 32 shows a system diagram in accordance with some embodiments.

FIG. 33A-33B show an exploded-view and an assembled-view of a chip card (e.g., device), respectively in accordance with some embodiments.

FIGS. 34A-34B show scanning electron microscopy (SEM) images of a top-view of a capture site, including the well, and a tilt-view of the capture site in accordance with some embodiments.

FIGS. 35A-35B show a top-down view of a capture site and a tilt-view of a capture site in accordance with some embodiments.

FIGS. 36A-36B show top-down views and labeled description of the port's (e.g., aspiration via's) of a capture site in accordance with some embodiments.

FIG. 36C depicts a labeled cross section of a capture site and the port in accordance with some embodiments.

FIGS. 37A-37D show tilt-view SEM images of a capture site, focusing in on the projection (e.g., needle/static needle/needle-like projection) in accordance with some embodiments.

FIGS. 38A-38D show top-down view diagrams of the device in accordance with some embodiments.

FIGS. 39A-39C show additional top-down view diagrams of the device in accordance with some embodiments.

FIGS. 40A-40B show top-down view diagrams of capture site packing patterns in accordance with some embodiments.

FIGS. 41A-41C show cross sectional diagrams of the wafer used to make the device in accordance with some embodiments.

FIGS. 42A-42C show various diagrams of a device having a back side aspiration port in accordance with some embodiments.

FIGS. 43A-43M depict a process for fabricating a gene transfection device in accordance with some embodiments.

FIGS. 44A-44H depict a process for fabricating a gene transfection device in accordance with some embodiments.

FIGS. 45A-45B depict various port boundary shapes in accordance with some embodiments.

### DETAILED DESCRIPTION

Systems, devices and methods for the delivery of payloads to large numbers of cells may have vast applications for research and development, diagnostics and therapeutics. For example, stem cell-based therapies benefit from the ability to efficiently transfect large numbers of cells with genetic material or genome editing tools, while maintaining cell viability.

Provided herein are methods and systems for precision mechanoporation. In some embodiments, precise mechanoporation may comprise capturing a plurality of cells, mechanically creating pores in the cells, and delivering or injecting a cargo into the cells through the pores. Delivering a cargo into a cell or facilitating the delivery of a cargo into a cell may be performed in a variety of forms and/or using a variety of techniques. For examples, cargo delivery may comprise cargo injection, cargo diffusion (i.e., the diffusion of the cargo into the cell), delivery of cargo with the aid of electrostatic charge or other suitable forms of cargo delivery. Cargo diffusion may in some cases comprise passive cargo diffusion. In some examples, any combination of the aforementioned techniques may be used for cargo delivery. In some examples, pores may be generated in the cells to deliver/inject a cargo into a section, subsection, component, constituent, or organelle of the cell. In some examples, the cargo may be injected/delivered to the nucleus of the cell. In some examples, a cargo may be injected into the cell to transfect the cell.

In an aspect, provided herein is a system for precision single-cell mechanoporation. The system may comprise a static array of wells. In some examples, each well of the array of wells may be configured to capture a single cell therein. In some cases, a single cell may be trapped in a well of the static array of wells via sedimentation (e.g., gravitational sedimentation) and flow-focusing of the single cell into the well. In some examples, the array of wells may be positioned horizontally. The wells may be in the bottom of the system on a horizontal surface or substrate. Alternatively, in some examples, the array of wells may be positioned in any intended position, orientation, or angle. For example, the array of wells may be positioned vertically. In some examples, the array of wells may be upside down (e.g., in the top surface of a microfluidic device/chip as opposed to its bottom surface). In some examples, the array of wells may be positioned at an angle of at least about 5, 10, 20, 30, 40, 45, 50, 60, 70, 80, 90, 100, 120, 130, 140, 150 degrees or larger relative to the horizontal plane. The positioning, orientation, and angle of the wells and/or array of wells may be adjustable before or during device operation and may be optimized based on the application and/or use case.

A single cell solution/suspension may be loaded into the system and set to flow therein. The suspension may comprise an aqueous solution (e.g., a buffer) with a plurality of cells suspended therein. Preferably, the cells in the solution may be substantially single cells, that is, the cells may not be preferred to be clumped together, although, some cell clumps may be present in the cell suspension or solution. The buffer compositions, cell density, and other compositions and properties of the solution may be adjusted to optimize the condition of the sample for performing the process. An optimized condition may comprise minimal to no cell clumps present in the solution.

Single cells may be trapped, captured, and/or encapsulated in single wells. A single cell may be trapped in a single well of the static array of wells. In some cases, such trapping may be performed as a result of or upon gravitational sedimentation of the single cell in the well. Once the cell travels down (sediments) down onto the well and focuses into the well and onto the needle/projection. This process may be referred to as cell flow-focusing because the flow (e.g., the pressure-driven flow in the microfluidic device or the pressure-driven microfluidic flow) focuses the cell on the needle, without the need for centrifugation of the traps or the system to lead the cells into the wells. Microfluidic flow may comprise or be a flow of fluid or a fluid flowing in microfluidic devices mentioned anywhere in this disclosure. The bottom of the trap may comprise a static needle which may be configured to precisely inject a cargo into the nucleus of the single cell which is captured/trapped in the well, in some examples, without back-and-forth movement of the needle.

The system may comprise one or more modules for generating a pressure gradient, wherein the pressure gradient is configured to induce a pressure-driven flow of a single cell suspension in the system, thereby leading to gravitational sedimentation, flow-focusing, and capture of the single cell in the trap. In some examples, capturing the single cell in the trap is accomplished without centrifugation of the system or the static array, and injection may be performed without moving the needle.

In some examples, a constant flow rate may be induced in the system. Alternatively, a constant pressure may be set, and the flow may self-adjust accordingly. Pressure-driven flow may comprise a few advantages over constant set flow rate, including higher stability, less system hiccups, lower failure rates, and shorter system ramp-up time leading to a shorter total cycle time, less sample waste, and higher overall throughput.

In some examples, the acts of cell capturing and cell injection (e.g., mechanoporation) as described anywhere herein may be performed without the need for or reliance on centrifugation and/or needle movement. For example, in some embodiments, the system including the array of the traps is not centrifuged in order to facilitate the capturing of the cell in the well. Instead, the cell will flow as a result of flow movement in the system to enter the well and land on the needle. The flow may be induced in the system by generating a pressure gradient (e.g., pressure-driven flow). Pressure may be induced in the system using one or more modules which may create positive and/or negative pressures in device ports, such modules may comprise pumps and vacuum sources which may be connected to the device ports, and which are further described anywhere herein. Flow as induced by a pressure differential may assist the cell to sediment on the well, into the well, and on the needle inside the well. Such process may be referred to as cell gravitational sedimentation and flow focusing, as the forces of gravity and fluid flow (e.g., generated by a pressure differential) may lead the cell into the well and on the needle.

Performing the aforementioned processes and steps without relying on centrifugation may be advantageous. Eliminating the need for centrifugation significantly simplifies the system compared to traditional mechanoporation systems which may rely on centrifugation for cell trapping/capturing. A system with a centrifuge may be more complex, more expensive, more prone to failure, less amenable to streamlining, parallelization, scaling, and automation. The devices and systems presented herein may be significantly simplified, more efficient, less prone to failure, more amenable to parallelization, and automation, at significantly lower price points. Therefore, designing a system which can effectively and efficiently perform cell capturing and mechanoporation with significantly simplified and automatable components is highly valuable. The device is comprised of a smaller number of modules and moving parts, is easier to build, and transport, may be lighter in weight leading to the convenience of transport, and may comprise additional advantages compared to a complicated system which relies on centrifugation for cell capturing. In order for this process to be successfully accomplished however, the system geometries and operational conditions may need to be optimized, so that cell capturing can be conveniently performed without centrifugation. Various embodiments of the system which may facilitate such cell capturing are disclosed throughout this disclosure.

The needle/projection may inject, deliver, or facilitate a delivery of a cargo or load into the cell, in some cases, precisely into the nucleus of the cell, such as to transfect the cell. A precise and localized insertion of the cargo into the nucleus of the cell may be important for the applications of the system (e.g., successful transfection and gene delivery). Delivery may be any kind of delivery mentioned anywhere in the disclosure.

The details of the geometries of the wells may be important for their cell capturing efficiency. As previously described, in some examples, the cell capturing and mechanoporation described in the present disclosure may be performed without reliance on centrifugation and/or the movement of the needle, in some cases, merely by relying on cell sedimentation and flow focusing into the well and on the needle. In order for such process to be made possible or made efficient, the geometries of the wells may need to be optimized. The flow rates and densities of the cell suspension in the system may need to be optimized. The pressures used to induce pressure-driven flow in the system may be optimized. Optimal geometries and operational conditions of the device are detailed throughout this disclosure.

The optimal geometries for performing the methods of the present disclosure may be identified. Upon identification and design of such geometries, devices comprising such geometries should be fabricated and manufactured. In some cases, such fabrication and manufacturing processes may need advanced skills and techniques and may be technically challenging. The present disclosure further details methods of fabrication and manufacturing of the described devices. As an example, in some cases, it may be intended to reduce the size of given device features (e.g., well diameters or well depths (i.e., a thickness of a device layer)). Precise fabrication of ultra-small, miniaturized features may be challenging and may require or benefit from advanced fabrication techniques. In some cases, defined deposition and etching techniques may be used for fabricating the devices of the present disclosure. The fabrication techniques used are described in detail elsewhere herein.

In some examples, the system may be capable of processing at least about 500, 600, 700, 800, 900, 1000, 1500, 2000, 3000, 4000, 5000, 6000, 7000, 8000, 9000, 10000, 15000, 20000, 30000, 40000, 50000, 60000, 70000, 80000, 90000, 100000, 150000, 200000, 210000, 220000, 230000, 240000, 250000, 260000, 500000, 600000, 700000, 800000, 900000 or more cells per cycle. In some examples, the system may be capable of processing at least about 0.5 million cells per cycle. In some examples, the system may be capable of processing at least 1 million cells per cycle. In some examples, the system may be capable of processing at least 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100 million cells per cycle or more. In some examples, the system may further comprise a computer software for controlling the performance of the system.

In some cases, each single cell may be processed in a single well in each cycle. Therefore, for example, in some cases, a device comprise a certain number of wells may process about that same number of cells per cycle. For example, if a device comprises 10000 wells, it may process about 10000 cells per cycle. In some cases, a device of the present disclosure may comprise at least about 500, 600, 700, 800, 900, 1000, 1500, 2000, 3000, 4000, 5000, 6000, 7000, 8000, 9000, 10000, 15000, 20000, 30000, 40000, 50000, 60000, 70000, 80000, 90000, 100000, 150000, 200000, 210000, 220000, 230000, 240000, 250000, 260000, 500000, 600000, 700000, 800000, 900000 or more wells. In some examples, the device may comprise at least 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100 million wells or more.

In some examples, the static array of wells may comprise at least 1,000 wells, and the system may be capable of processing at least about 1,000 cells per cycle. In some examples, the static array of wells may comprise at least 2,000 wells, and the system may be capable of processing at least about 2,000 cells per cycle. In some examples, the static array of wells may comprise at least 15,000 wells, and the system may be capable of processing at least about 15,000 cells per cycle. In some examples, the static array of wells may comprise at least 20,000 wells and the system may be capable of processing at least about 20,000 cells per cycle. I some examples, the static array of wells may comprise at least 100,000 wells and the system may be capable of processing at least about 100,000 cells per cycle. In some examples, the static array of wells may comprise at least about 100,000 wells, and the system may be capable of processing at least about 100,000 cells per cycle. In some examples, the static array of wells may comprise at least 200,000 wells and the system may be capable of processing at least about 200,000 cells per cycle. Accordingly, the static array of wells may comprise any number of wells mentioned anywhere herein and be capable of processing any number of cells processed per cycle which is mentioned anywhere herein. For example, the static array of wells may comprise at least 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100 million wells and be capable of processing at least about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100 million cells per cycle.

In some examples, the cell may be captured in the well and mechanoporated as described anywhere herein without the need for the needle/projection to move. The terms needle and projection may be used interchangeably throughout this disclosure. A static non-moving needle may comprise a variety of advantages over a system with a moving needle. Moving and operating the needle may significantly complicate the system. Performing the applications of the present disclosure (e.g., trapping and mechanoporation of the cells) without moving the needle may significantly simplify the system. A system without centrifuge and moving needle may be significantly simpler, more convenient, cheaper, more efficient, easier to transport, more amenable to scaling, parallelization, and automation, compared to a system with one or more of both of centrifugation and/or a moving needle/projection required for mechanoporation and/or intracellular cargo delivery. Moreover, fabricating the systems of the present disclosure which in some examples do not comprise a moving needle and a dynamic centrifuge system may be performed more efficiently than more complicated systems, such that the failure rates of the manufacturing processes may be significantly lower in the systems of the present disclosure. The methods of fabrication and manufacturing of the system described in this section is disclosed in detail, elsewhere in the disclosure. Such manufacturing processes are compatible with generating a trapping array of wells each of which comprises a static needle in the bottom of the well.

In some examples, the methods and systems of the present disclosure facilitate performing the poration of individual cells in a population of single cells, such that the processed cells are treated in a substantially similar manner to each other. In this way, the poration and cargo delivery is substantially uniform across the processed cell population. In some cases, precision incision in each cell minimizes damage to the processed cells and provides a path for delivery to the nucleus of the cell. The absence of moving parts in the device/system may reduce/minimize complexity and enable massive parallelization, thus enabling the opportunity for scaling to higher mechanoporation and intracellular delivery throughputs which may be highly beneficial for cell and gene therapy (CGT) applications, among other applications.

The methods and systems of the present disclosure may comprise a unique an unprecedented ability to safely and precisely produce a singular pore in both the plasma and nuclear membranes in single cells among a large population of cells. This may lead to a high intracellular and/or intranuclear cargo delivery efficiency as well as a high cellular viability, upon processing. The methods and systems may facilitate direct intranuclear delivery of cargoes into cells while maintaining the viability and integrity of the cells.

In some cases, the treatment of the cells in the population may be substantially uniform. Many pre-existing non-viral techniques (e.g., electroporation, shear poration, chemical poration, and other techniques) typically suffer from poor uniformity across the cell population in terms of poration and cargo delivery and may often force users to choose between efficiency or viability. This may at least in part be due to their reliance upon inherently random poration mechanisms, and their inability to reliably and/or precisely breach the nuclear envelope.

In an aspect, provided herein is a method of parallelized deterministic mechanoporation for intracellular cargo delivery. The method may comprise (a) providing or obtaining a mechanoporation system. The mechanoporation system may comprise (i) a static array of wells, wherein each well of the static array of wells is configured to capture a single cell therein via sedimentation (e.g., gravitational sedimentation) and flow-focusing of the single cell as described anywhere throughout the present disclosure. The mechanoporation system may further comprise (ii) a static needle in the bottom surface of each well of the static array of wells. The mechanoporation system may further comprise one or more modules for generating a pressure gradient in the system. The mechanoporation system may further comprise one or more liquid reservoirs for holding the samples to be processed which may contain a cell suspension for mechanoporation and cargo delivery.

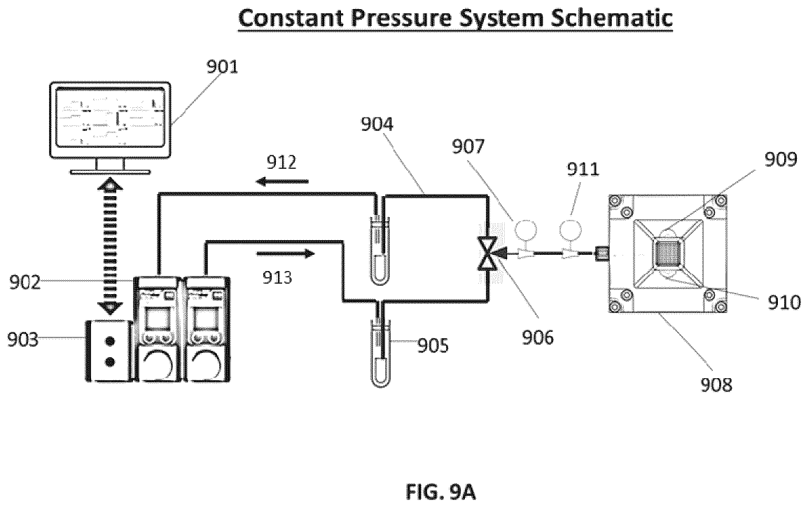

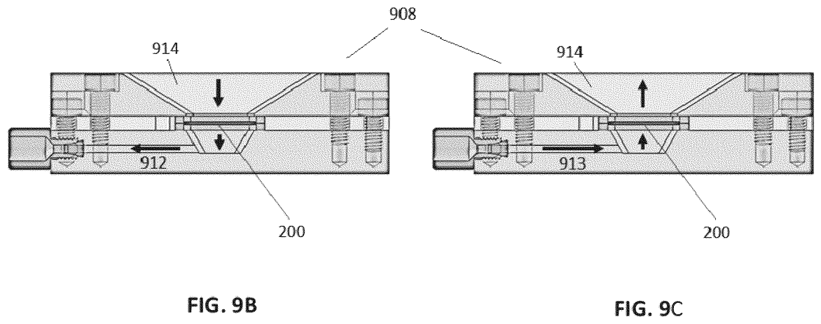

The method may further comprise providing or obtaining a cell suspension in the one or more liquid reservoirs of (iv), generating a pressure gradient across the one or more liquid reservoirs of (iv) and the static array of wells of (i) using the one or more modules of (iii), thereby generating a pressure-driven flow of the cell suspension from the reservoirs across the static array of wells. The method may further comprise capturing single cells from the cell suspension in the wells of the static array of wells, without centrifugation, thereby generating a single captured cell in the well. In some cases, the method may be performed using the system of FIG. 9A. An example may be Example 14.

The method may further comprise creating a pore in the single captured cell, and delivering a cargo into the nucleus of the single captured cell using the static needle of (ii). The cargo delivered into the single captured cell may in some cases be intended for transfecting the cell.

The methods and systems of the present disclosure may facilitate maintaining the viability of the processed cells. For example, the single captured cell may remain viable after performing the method thereon. In some examples at least about 20%, 30%, 40%, 50%, 60%, 70%, 80%, 85%, 90%, 95%, 99% or more of the population of cells processed using the method may remain viable after such processing.

The methods and systems of the present disclosure may facilitate achieving a high throughput, such that a large number of cells may be processed per unit time or per cycle. The number of total cells processed per cycle and/or per unit time may at least partially depend on the efficiency of cell capturing, cell capturing efficiency, and/or single cell capturing efficiency.

Typically, once a population of cells are introduced into the system, a sub-population/portion of the wells of the static array of wells may capture a single cell, a portion of the wells may remain empty, and a portion of the wells may capture more than one cell. In some cases, the distribution of the cells in the wells may follow a Poission or super-Poisson distribution. It may be desired to maximize the number of wells containing single cells and minimize the number of wells containing multiple cells. The methods and systems of the present disclosure may facilitate cell capturing at a superior single cell capturing efficiency. In some examples, at least about 10%, 11%, 15%, 20%, 25%, 30%, 35%, 40%, 50%, 60%, 70%, 80%, 90% or more of the wells of the static array of wells may capture a single cell therein upon performing the method.

In some examples, the cargo load delivered into individual cells among a population of cells processed using the method may be substantially uniform across the population of cells processed. Stated in different words, cell-to-cell variability among the single cells processed using the method, in terms of the amount of cargo delivered to them or to their nuclei may be low. Cargo load may be measured and characterized across the population. In some cases, cargo loads may be characterized by detecting a fluorescent signal (e.g., fluorescent) from each of the processed cells. Once the signals are detected, a coefficient of variance or coefficient of variability (% CV) may be used to characterize the uniformity of the cargo delivery across the population of cells. The coefficient of variability may be calculated as the standard deviation value of the signal indicative of the cargo load divided by the mean value of the same signal. The signal may be a fluorescent signal. In some examples, the signal may be captured via imaging and quantified via image processing.

In some examples, a coefficient of variability (% CV) across individual cells among a population of cells processed using the mechanoporation method may be at most about 70%, 50%, 40%, 30%, 20%, 15%, 10%, 9%, 8%, 7%, 6%, 5%, 4%, 3% or less.

The precision mechanoporation method may be performed without the need for centrifugation. The method may be performed without a dynamic needle moving up and down or back and front. The system may be substantially simple. The substantially simple system may be highly efficient to manufacture and operate at very low failure rates. The simple system may allow for achieving a high single cell capturing and cell viability efficiency as described in the preceding paragraphs. In some cases, such high levels of performance metrics may be achieved due to the optimized geometry of the device, the geometry of the wells, the operational conditions of the device (e.g., set pressures for inducing pressure-driven flow), buffers used in the cell suspension, and the improved fabrication technologies detailed throughout the disclosure.

The methods and systems described in the preceding paragraphs are further elaborated on in great detail in the following paragraphs. The geometries of the device are illustrated in the figures and examples. For instance, an exemplary constant pressure mechanoporation system is detailed in Example 14 and schematically illustrated in FIG. 9A.

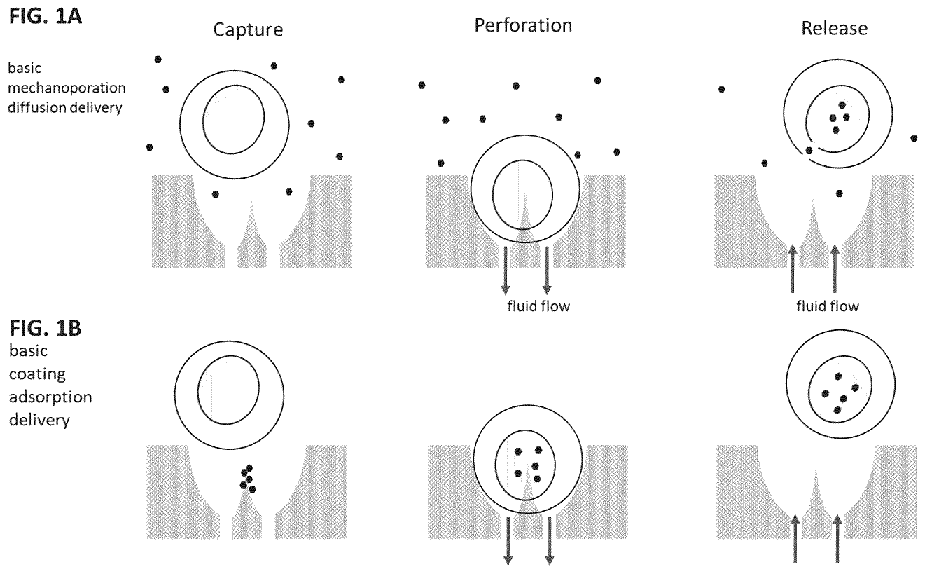

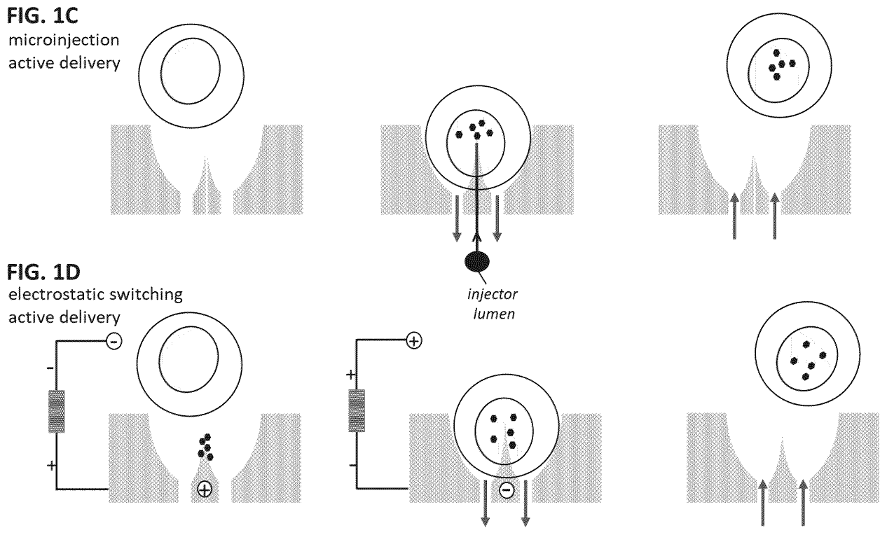

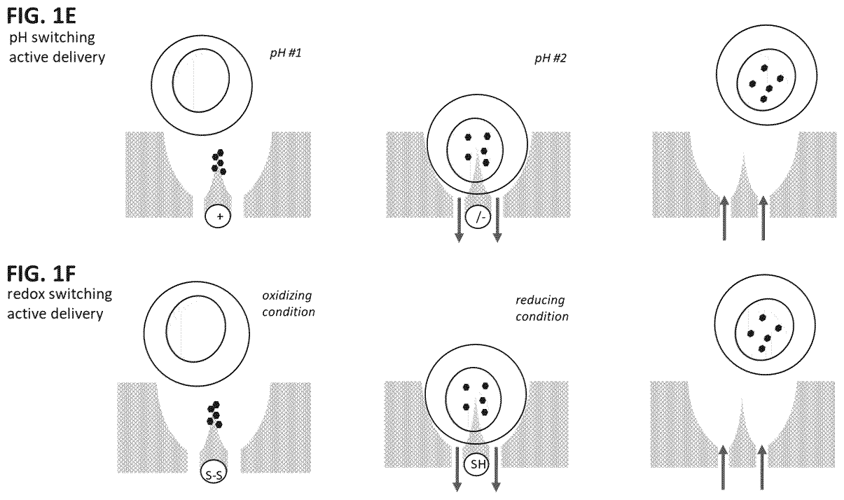

Provided herein are various embodiments of systems, devices, compositions and methods for payload insertion into cells. In some examples, systems, devices, compositions and methods described herein provide for the capture of, perforation (e.g., puncture, poration, or breach) of, release of and payload insertion to cells, as shown in FIGS. 1A-1F. Such features may be configured to work with various cell types and may deliver cargos (e.g., payloads) to cells, in some cases, to the nucleus of the cells.

In certain examples, systems, devices, compositions and methods described herein may provide for or facilitate the transfection of a large number of cells. In some cases, a large number of cells may be processed in a cycle of device run. A device cycle may be the duration of time from starting the experiment on the system, once all the system components have been set up until the end of the experiment (e.g., stopping and shutting off the run). The run cycle may depend on the intended experiment.

The run cycle may be determined and adjusted as needed, on a case-by-case basis, for example, for an intended experiment. The run cycle may depend on sample size, such as the number of cells that are to be processes. In some cases, a large number of cells can be processed in unit time. The number of cells process in unit time may be referred to as the run throughput. In some cases, experiments may comprise or be high-throughput experiments. High-throughput experiments may facilitate processing a large number of cells in a short time.

In some examples, the number of cells processed per cycle may be large. In some examples, cell processing may comprise cell transfection. In some examples, the number of cells processed in a cycle may be at least about 1000, 2000, 5000, 10000, 100000, 200000, 250000, 300000, 400000, 500000, 1 million or more cells/cycle. In some examples, the number of cells processed per cycle may be at least about 1, 2, 3, 4, 5, 6, 7, 8, 9, 10, 20, 30, 40, 50, 60, 70, 80, 90, 100, 200, 300, 400, 500, 600, 700, 800, 900, or more cells/cycle.

In some embodiments, a cycle may be referred to as a process whereby the capture of, perforation of, release of and payload insertion to cells is performed on the device or by the system. In some embodiments, following release of perforated cells, the device or system may be used for additional cycles. As shown in FIGS. 1A-1F, perforation may be utilized to produce a pore (e.g., hole) in each cell, thus enabling delivery of cargo directly to the interior of the cell and/or nucleus without damaging the cells and maintaining cell viability.

In some embodiments, as shown in FIG. 1A-1F, operations of various methods described herein may include capture of, perforation of, release of, and insertion of a payload into the cell. In some embodiments, various methods for perforation and insertion (e.g., delivery) of a payload may comprise mechanoporation diffusion delivery of a payload as depicted in FIG. 1A. In some embodiments, various methods for perforation and insertion (e.g., delivery) may comprise adsorption delivery of a payload as depicted in FIG. 1B. In some embodiments, various methods for perforation and insertion (e.g., delivery) may comprise microinjection active delivery of a payload as depicted in FIG. 1C. In some embodiments, various methods for perforation and insertion (e.g., delivery) may comprise electrostatic switching active delivery of a payload as depicted in FIG. 1D. In some embodiments, various methods for perforation and insertion (e.g., delivery) may comprise pH switching active delivery of a payload as depicted in FIG. 1E. In some embodiments, various methods for perforation and insertion (e.g., delivery) may comprise reduction and oxidation (redox) switching active delivery of a payload as depicted in FIG. 1F.

In some embodiments, the operations of a method may take place in an environment filled with the payload. In some embodiments, the operations of a method may take place whereby the payload may be coated, adsorbed or localized onto the surface of the device. In some embodiments, the payload may be specifically coated or absorbed to wells of the device. In some embodiments, the payload may be specifically coated or absorbed to the projection (e.g., needle) of the device. In some embodiments the device surface or portion of the device is treated or coated with compounds or polymers to assist or modulate payload adsorption to the surface of the device. Payload may be nonspecifically adsorbed to the device, or in other embodiments, payload may be attached through specific reversible chemistry or charge interactions. In some embodiments, the payload may be adsorbed onto the device using reversible disulfide bond formation and is released into the cell by reduction of the disulfide bond. In some embodiments the payload is adsorbed onto the surface of the device in a pH dependent manner and is released to the cells in a pH dependent manner. In some embodiments the payload is adsorbed onto the surface of the device in an electrochemical charge dependent manner and is released to the cell by reversing the electrochemical charge applied to the device. In some embodiments the payload is delivered to the cell using microinjection into the cells from the device capture site, including the device wells and projections (e.g., needles). In some embodiments, the payload comprises genetic material. In some embodiments, the genetic material comprises target genes. In some embodiments, the payload comprises gene/genome editing components or tools. In some embodiments, at capture, the cells are distributed into arrayed wells on the device using fluid flow. In some embodiments, fluid flow is controlled in a constant flow manner. In some embodiments, fluid flow is controlled in a constant pressure differential manner. In some embodiments, at perforation (e.g., poration), the cell may be perforated with a single perforation. In certain aspects, at release, the perforated cell may be placed back in the environment for the insertion of the target gene (e.g., “cargo,” “payload” or “genetic material”) into the cell. In some embodiments, at perforation, the insertion of the target gene (e.g., “cargo,” “payload” or “genetic material”) occurs during cell perforation.

In the current art, performance limitations of prevailing gene delivery techniques may include prevention of full realization of the Cell and Gene Therapy (CGT) industry's therapeutic promise. Currently, two limited capability methods are suitable and currently employed for therapeutic ex vivo gene delivery applications: electroporation and viral vectors.

In the current art, issues involving Viral Vector Transduction include: (1) poor efficiency, particularly for stem and primary human cells; (2) limited versatility, including packaging constraints; (3) limited scalability due to high production cost and long lead times; and (4) safety concerns, including insertional oncogenesis.

In the current art, issues involving electroporation include: (1) poor efficiency, particularly for stem and primary human cells; (2) limited versatility, including challenges with delivery of large cargo to the nucleus; (3) limited scalability, including uniformity challenges; and (4) safety concerns, including functional deficits.

In some embodiments, a nanomechanical method comprising precise (non-stochastic like electroporation) may be used for gene delivery. In some embodiments, mechanoporation allows for: (1) uniform single-site perforation in the cell; (2) nuclear delivery; (3) massive parallelization; (4) rapid cell processing; and (5) simplified operation.

In the current art, delivery of model cargos that require cytosolic localization (e.g., GFP mRNA) may find a smaller application space. The performance of these alternative methods is likely lower for delivering large cargos and may struggle to achieve required nuclear localization

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

### Applications and Reagents

Described herein are various systems, methods, reagents and devices for transfecting suspended cells, specifically designed to meet the needs of engineered cell product manufacturing. In some embodiments, systems, devices, reagents and methods are configured for delivery of various cargos to cells. In some embodiments, applications for delivery of various cargos to cells may comprise cellular engineering, gene transfection, genetic deletion, genetic addition, genetic engineering or genetic modification. In some embodiments, methods include deterministic mechanoporation (DMP) of large numbers of cells, each at a single site in their plasma membrane, the methods may allow rapid collection of the cells for subsequent processing. In some embodiments, DMP enables efficient delivery of large-molecule cargos while minimizing damage to the cell, thus allowing achievement of transfection yields that exceed current non-viral transfection techniques. DMP provides a mechanism for addressing roadblocks in the development and manufacture of ex vivo cell therapies based on engineered T cells (e.g., adoptive cancer immunotherapies based on chimeric antigen receptor modified T cells or engineered T cell receptor (TCR) therapies). Applications, systems, devices and methods for delivery of payloads to cells are described in Balas et al. U.S. Pat. No. 9,885,059B2 and Rao et al. US20200289568A1, wherein both references are herein incorporated by reference in their entirety.

In some embodiments, methods may comprise primary human T cell engineering. In some embodiments, methods may utilize model cargos that require nuclear localization (e.g., GFP plasmid). In some embodiments, applications may comprise HDR-mediated large gene insertion.

### Cells

In some embodiments, the systems devices and methods may provide for the mechanoporation of various cell types. In some embodiments, systems, devices and methods may be used with various cell types. In some embodiments, systems, devices and methods may be used with any cell type, wherein the cell type may be prepared as a suspension of single cells. In some embodiments, protoplasts prepared from cells may be used. In some embodiments, the cell preparation (e.g., suspension of cells) may be exposed to isotonic, hypotonic or hypertonic conditions to modulate cell size. In some embodiments, cell origins may comprise human, animal, plant or a combination thereof. In some embodiments, cell types may comprise T cells, Natural Killer Cells (NK Cells), NKT Cells, Monocytes, Myeloid Cells, Hematopoietic Stem Cells, Mesenchymal Stem Cells, Induced Pluripotent Stem Cells (iPSC), Hepatocytes, or Progenitor cells.

### Payload and Surface Coatings

In some embodiments, various payloads may be inserted into a cell. In some embodiments, payloads may comprise chemical compounds (organic or inorganic), proteins, peptides, polymers and nucleic acids. In some embodiments, various payloads may be inserted into a cell as a single agent. In some embodiments, various payloads may be inserted into a cell as mixtures. In some embodiments, the payload DNA may comprise any form of natural or synthetic DNA, including genomic DNA, viral DNA, cDNA, plasmid DNA, dsDNA, ssDNA, oligonucleotides and hybrid sequences or synthetic or semi-synthetic sequences thereof. In some embodiments, the payload RNA may comprise any form of RNA, including viral RNA, RNAi, siRNA, saRNA, mRNA, tRNA, rRNA, microRNA, gRNA, sgRNA and hybrid sequences or synthetic or semi-synthetic sequences thereof. In some embodiments, the payload proteins may comprise enzymes including CRISPR/Cas9 alone or in complex (RNP) with guideRNA or sgRNA complexes for gene editing using CRISPR. In some embodiments, payloads may comprise alternative class enzymes for CRISPR (other than Cas9). In some embodiments, payload proteins may comprise enzymes such as transposases including Sleeping Beauty transposase and PiggyBac transposase. In some embodiments, the payload may comprise TALENS and other synthetic hybrid molecules such as Cas-CLOVER. In some embodiments, payload proteins may comprise transcription factors natural or synthetic mediating activation or repression of gene expression. In some embodiments, payload proteins or peptides may comprise synthetic or mutated. In some embodiments, the payloads may be delivered in suspension as depicted in FIG. 1A. In some embodiments, the payloads may be present in solutions before, during, and/or after the perforation event. In some embodiments, the payloads may be formulated with condensation agents. In some embodiments these condensation agents may comprise proteins or peptides (protamine, neuphorin, etc.), lipids including cationic lipids (Lipofectamine, dimethyldidodecylammonium bromide, didecyldimethylammonium bromide, dimethyldioctadecylammonium bromide, etc), or polymers including cationic polymers (polyethyleneimine, etc.). In some embodiments, commercial transfection enhancers or transfection agents may be used. In some embodiments, the payload may be injected into the cell through a lumen located within a projection/needle as depicted in FIG. 1C. In some embodiments, the payload may be adsorbed onto the device, well, and/or projection/needle 209, as depicted in FIGS. 1B, 1D-1F.

In some embodiments, the payload may be adsorbed onto a coating applied to the device, well, and/or projection/needle. In some embodiments the coating may comprise a positively charged molecule, negatively charged molecule, or a molecule of mixed charge. In some embodiments, the coating may be applied as a mixture. In some embodiments, coatings may be applied in series. In some embodiments, the coating comprises a polymer. In some embodiments, the polymer comprises poly-L-lysine, poly-D-lysine, poly-L-arginine, poly-D-arginine, poly-L-ornithine, poly-D-omithine, poly-L-histidine, poly-D-histidine, poly-L-glutamate, poly-D-glutamate, alginate, hyaluronic acid, polyethyleneimine (PEI), polybrene, chitosan, pullulan, or poly(diallyldimethylammonium chloride). In some embodiments, the coating comprises block copolymers of the polymer with polyethylene glycol (PEG). In some embodiments, the coating comprises block copolymers comprising polyethylene glycol (PEG). In some embodiments, the PEG comprises poly-L-lysine-g-PEG (e.g., PLL-g-PEG). In some embodiments, the coating comprises mixtures of the polymer or coblock polymer. In some embodiments, the coating may comprise lipids. In some embodiments, the coating comprises cationic lipids such as Lipofectamine, dimethyldidodecylammonium bromide, didecyldimethylammonium bromide, or dimethyldioctadecylammonium bromide. In some embodiments, the coating may comprise polyhistidine containing polymers or mixtures. In some embodiments, the coating may comprise polyhistidine containing polymers or mixtures configured to allow for pH dependent cargo adsoption and release in cell compatible/physiological pH range. In some embodiments, the pH dependent payload (e.g., cargo) adsorption and release in a cell compatible/physiological pH range may be mediated by compounds other than histidine. In some embodiments, the coating comprises a protein. In some embodiments, the coating comprises protamine. In some embodiments, the coating comprises a functionalized silane. In some embodiments, the functionalized silane comprises an amino terminated silane. In some embodiments, the amino terminated silane comprises 3-Aminopropyltriethoxysilane (APTES), Trimethoxysilylpropyldiethylenetriamin (DETA), N-(2-aminoethyl)-3-aminopropyltrimethoxysilane (EDA), m,p-(aminoethyl-aminomethyl)phenethyltrimethoxysilane (PEDA), or a silane-PEG-amine. In some embodiments, the coating comprises a sulfhydryl terminated silanes. In some embodiments, the sulfhydryl terminated silane comprises 3-(mercaptopropyl)trimethoxysilane (MPTES/MTS) or mercaptomethyldimethylethoxysilane (MDS). In some embodiments, the coating comprises polyethyleneglycol (PEG) silanes. In some embodiments, the PEG-silanes comprise Methoxy-PEG-triethoxysilane (m-PEG-silane). In some embodiments, the coating comprises an inorganic material. In some embodiments, the coating comprises a metal. In some embodiments, the metal may comprise gold, zinc, or nickel. In some embodiments the coating may comprise a nitride material. In some embodiments, the coating may comprise carbon. In some embodiments, the coating may be applied by bulk adsorption, sputtering, evaporation, spotting, printing, or electrodeposition methods.

### Other Reagents

In some embodiments, system, devices, kits, or methods may comprise a buffer. In some embodiments, the buffer may comprise Phosphate Buffered Saline (PBS), Dulbecco's Phosphate Buffered Saline (DPBS), Hanks Balanced Salt Solution (HBSS), or HEPES.

In some embodiments, system, devices, kits, or methods may comprise media. In some embodiments, the media may comprise OptiMEM (Gibco), RPMI, X-Vivo15 (Lonza), X-Vivo20 (Lonza), CTS Optimizer T Cell Expansion SFM (Thermo), CTS AIM V Medium (Thermo), CTS KnockOut SR XenoFree Medium (Thermo), or CTS StemPro MSC SFM (Thermo).

In some embodiments, system, devices, kits, or methods may comprise serum and serum replacements. In some embodiments, the serum and serum replacements may comprise FBS, FCS, Human Serum, BSA, HSA, or CTS Immune Cell Serum Replacement (Thermo).

In some embodiments, system, devices, kits, or methods may comprise a surfactant. In some embodiments, the surfactant may comprise F-68 (Poloxamer 188, Kolliphor P 188, Lutrol F68, Pluronic F68, Synperonic F68). In some embodiments, the surfactant may comprise F-108 (Poloxamer 338, Pluronic F108, Synperonic F108). In some embodiments, system, devices, kits, or methods may comprise antioxidant, anti-apoptotic or cell protective components. In some embodiments, the antioxidant, anti-apoptotic, or cell protective component may comprise vitamin E, TROLOX, dexamethasone, RHO/ROCK inhibitors (such as Y27632, thiazovivan), pyruvate, ascorbate, or n-acetylcysteine (NAC). In some embodiments, system, devices, kits, or methods may comprise a transfection enhancer. In some embodiments, the transfection enhancer may inhibit or modulate cytosolic nucleic acid sensors such as Absent in Melanoma 2 (AIM2) and cyclic GMP-AMP synthase (cGAS) or inhibit or modulate toll-like receptor (TLR) signaling pathways such as TLR7 and/or TLR9. In some embodiments, these transfection enhancers may comprise glycerol, DMSO, NATE (InvivoGen), K4 Multiplier (Biontex), Trans-Booster (RJH Bioscience), chloroquine, E4664 or AT791. In some embodiments, system, devices, kits, or methods may comprise compounds or treatments that increase the size of nuclear pores in the cell. In some embodiments the compounds or treatments that increase the size of nuclear pores in the cell may comprise dexamethasone.

### Device

### Introduction

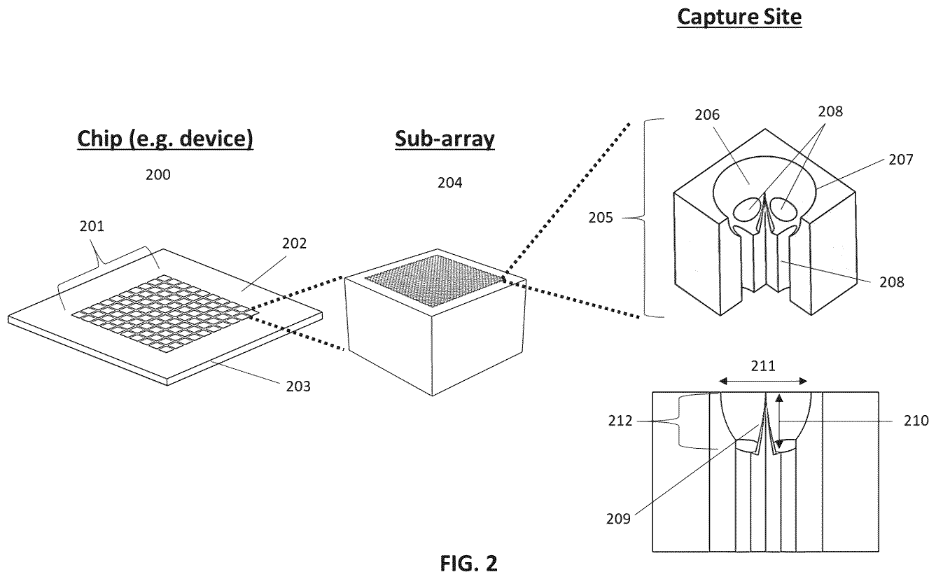

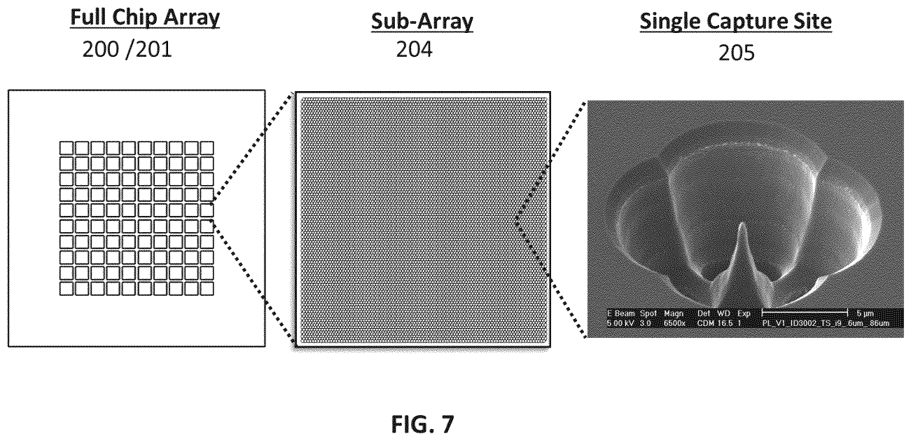

Described here are various embodiments of a device configured for inserting a payload into a cell. FIG. 2 shows an example device construction. In some embodiments, the device 200 comprises an array of sub-arrays 201, a surface 202, a substrate 203, or a combination thereof. In some embodiments, the array of sub-arrays 201 may be referred to as an array or full array. In some embodiments, sub-arrays 201 may comprise a plurality of capture sites 205. In some embodiments, the capture site 205 comprises a well 206, a well boundary shape 207, a port 208, a projection/needle 209 or a combination thereof. In some embodiments, each capture site of the device may be configured for capture of a single cell. Shown in FIG. 2 is a non-limiting example of a single sub-array 204 comprising approximately 2372 capture sites. As an example, a full device 200, comprising approximately 100 sub-arrays and approximately 237,200 capture sites are shown in FIG. 2. In some embodiments, as depicted in FIG. 7 is a non-limiting example of a single sub-array 201 comprising approximately 10,000 capture sites 205. As an example, a full device 200 comprising an array of sub-arrays 201, the array of sub-arrays comprising approximately 100 sub-arrays 204 and approximately 1,000,000 capture sites 205 is shown in FIG. 7.

In some examples, a single sub-array array may comprise at least 1, 10, 100, 1000, 10000, 100000, 200000, 300000, 400000, 500000, 600000, 700000, 800000, or more capture sites. In some examples, the array may comprise at least 1 million, 2 millions, 3 millions, 4 millions, 5 millions, 10 millions or more capture sites. In some examples, a sub-array may comprise at most about 100 millions, 80 millions, 70 millions, 60 millions, 50 millions, 40 millions, 30 millions, 20 millions, 10 millions, 5 millions, 2 millions, 1 million, or less capture sites. In some examples, a sub-array may comprise at most about 800000, 700000, 600000, 500000, 400000, 300000, 200000, 100000, 50000, 40000, 20000, 10000, 5000, 2000, 1000, 900, 800, 700, 500, 200, 100, or less capture sites.

### Array of Sub-Arrays

In some embodiments, the array of sub-arrays 201 comprises at least 1 sub-array 204. In some embodiments, the array of sub-arrays 201 comprise about 100 sub-arrays 204. In some embodiments, the array of sub-arrays 201 comprise sub-arrays 204 arranged in a linear (e.g., square packed) pattern. In some embodiments, the device 200 comprises one array of sub-arrays 201. In some embodiments, device 200 comprises at least one array of sub-arrays 201. In some embodiments the device 200 comprises two or more array of sub-arrays 201 arranged on one or more surfaces 202 of the device 200.

In some examples, the array of sub-arrays 201 may comprise at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 90, 100, 110, 120, 130, 140, 150, 200, 250, 300, 400, 500 or more sub-arrays. In some examples, the array may comprise at most about 1000, 900, 800, 700, 600, 500, 400, 300, 200, 100, 90, 80, 70, 60, 50, 40, 30, 20, 10 or less sub-arrays.

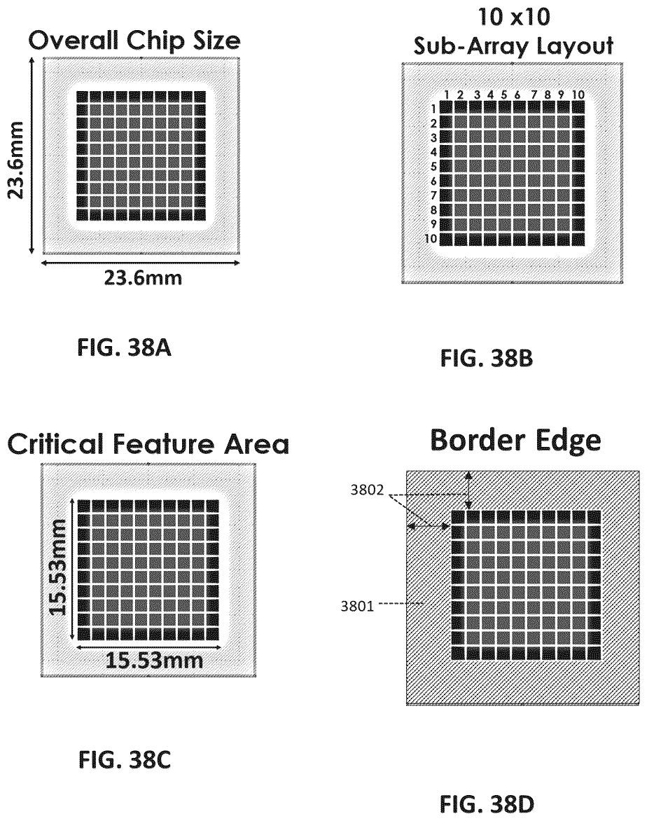

In some embodiments, the device (e.g., overall chip or full chip) 200 size is defined in terms of the edge length by the edge width of the device. In some embodiments, the device edge length is equal to the device edge width. In some embodiments, the length of the device edge length is 10 millimeters or less. In some embodiments, the device edge length 100 millimeters or more. In some embodiments, the length of the device edge length is about 10 millimeters to about 100 millimeters. In some embodiments, the length of the device edge length is about 20 millimeters to about 25 millimeters. In some embodiments, the length of the device edge length is about 23.1 millimeters to about 24.1 millimeters. In some embodiments, the device edge length is 23.6+/−0.5 millimeters for each of the four sides. In some embodiments, the device edge length (e.g., overall chip size), as shown in FIG. 38A, is defined as the length by width, which is shown to be 23.6 millimeters by 23.6 millimeters.

In some embodiments, the array of arrays (e.g., sub-array layout) 201 is defined as the number of sub-arrays 204 aligned horizontally across the device (e.g., overall chip or full chip) 200 and the number of sub-arrays aligned vertically across the chip. In some embodiments, as shown in FIG. 38B, the array of arrays (e.g., sub-array layout) 201 comprises an array of 10 sub-arrays by 10 sub-arrays. In some embodiments, the array of arrays may comprise an array of sub-arrays at least 2 sub-arrays aligned horizontally across the device. In some embodiments, the array of arrays may comprise an array of sub-arrays at least 100 sub-arrays aligned horizontally across the device. In some embodiments, the array of arrays may comprise an array of sub-arrays of 2 sub-arrays to 100 sub-arrays, aligned horizontally across the device. In some embodiments, the array of arrays may comprise an array of sub-arrays of 10 sub-arrays, aligned horizontally across the device. In some embodiments, the array of arrays may comprise an array of sub-arrays at least 2 sub-arrays aligned vertically across the device. In some embodiments, the array of arrays may comprise an array of sub-arrays at least 100 sub-arrays aligned vertically across the device. In some embodiments, the array of arrays may comprise an array of sub-arrays of 2 sub-arrays to 100 sub-arrays, aligned vertically across the device. In some embodiments, the array of arrays may comprise an array of sub-arrays of 10 sub-arrays, aligned vertically across the device.

In some embodiments, the sub-array comprises a critical feature area (e.g., active area). In some embodiments, the area of the device surface that contains the capture sites is referred to as the critical feature area. In some embodiments, the critical feature area (e.g., active area) is defined by the length of the sub-arrays aligned horizontally and the length of the sub-arrays aligned vertically. In some embodiments, length of the sub-arrays aligned horizontally is 10 millimeters or less. In some embodiments, length of the sub-arrays aligned horizontally is 20 millimeters or more. In some embodiments, the length of the sub-arrays aligned horizontally is about 15 millimeters to about 16 millimeters. In some embodiments, the length of the sub-arrays aligned horizontally is about 15 millimeters. In some embodiments, the length of the sub-arrays aligned horizontally is 15.53 millimeters. In some embodiments, length of the sub-arrays aligned vertically is 10 millimeters or less. In some embodiments, length of the sub-arrays aligned vertically is 20 millimeters or more. In some embodiments, the length of the sub-arrays aligned vertically is about 15 millimeters to about 16 millimeters. In some embodiments, the length of the sub-arrays aligned vertically is about 15 millimeters. In some embodiments, the length of the sub-arrays aligned vertically is 15.53 millimeters. In some embodiments, the critical feature area comprises a length of sub-arrays aligned vertically to be 15.53 millimeters by a length of sub-arrays aligned horizontally to be 15.53, as shown in FIG. 38C.

In some embodiments, the device comprises a border edge (e.g., border area) 3801 depicted as the hatched area of FIG. 38D. In some embodiments, the border edge comprises a border edge width 3802 also depicted in FIG. 38D. In some embodiments, the border edge width may be about 1 to about 10 millimeters. In some embodiments, the border edge width may be about 3 to 4 millimeters. In some embodiments, the border edge width may be 3.86 millimeters.

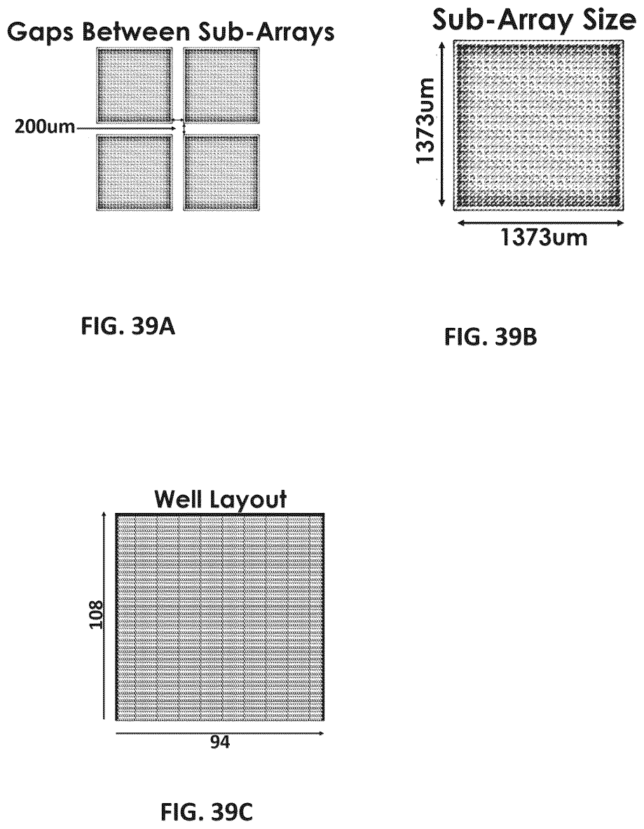

In some embodiments, the array of sub-arrays (e.g., sub-array layout) comprise a gap between sub-arrays. In some embodiments, the gap may be 1000 microns or more. In some embodiments, the gap may be less than 10 microns. In some embodiments, the gap is about 10 microns to about 1000 microns. In some embodiments, the gap is about 100 microns to about 300 microns. In some embodiments, the gap is 200 microns to 202 microns. In some embodiments, the gap is about 200 microns. In some embodiments, the gap is 200 microns +2, −0 microns. In some embodiments, as shown in FIG. 39A, the gap between sub-arrays is 200 microns.

### Sub-Arrays

In some embodiments, the device 200 comprises at least one sub-array. In some embodiments, the device 200 comprises about 1 sub-array 204 to about 140,000 sub-arrays. In some embodiments, the device comprises an array of sub-arrays 201, wherein the sub-arrays are arranged in an ordered fashion. In some embodiments, the device 200 comprises an array of sub-arrays, wherein the sub-arrays are arranged in a random fashion. In some embodiments, the device comprises an array of sub-arrays, wherein the sub-arrays are arranged in a linear fashion. In some embodiments, the sub-array comprises a capture site 205. In some embodiments, the sub-array comprises at least one capture site. In some embodiments, the sub-array may comprise from one capture site and about 2,000,000,000 capture sites. In some embodiments, the sub-array comprises 2,000,000 or more capture sites. In some embodiments, the sub-array comprises about 2372 capture sites. In some embodiments, the capture sites are arranged in the sub-array in a linear (e.g., square) pattern. In some embodiments, the capture sites are arranged in a diamond pattern (e.g., face centered, hexagonal or cubic centered) pattern. In some embodiments, the capture sites are arranged in a random pattern. In some embodiments, the capture sites of the sub-array are arranged in a hexagonal pattern.

In some embodiments, the sub-array size refers to the length of the sub-array by the width of the sub-array. In some embodiments, as depicted in FIG. 39B a sub-array comprises a sub-array size of 1373 microns by 1373 microns, where the width of the sub-array is the horizontal axis, and the length of the sub-array is the vertical axis. In some embodiments, the sub-array size (e.g., length by width) is 1373+10 microns, −0 microns and the width is 1373+10, −0 microns. In some embodiments, the length of the sub-array is about 10 microns or less. In some embodiments, the length of the sub-array is 10000 microns or more. In some embodiments, the length of the sub-array is about 10 microns to about 10000 microns. In some embodiments, the length of the sub-array is about 100 microns to about 5000 microns. In some embodiments, the length of the sub-array is about 1363 microns to about 1383 microns. In some embodiments, the length of the sub-array is about 1373 microns. In some embodiments the length of the sub-array is 1373 microns. In some embodiments, the width of the sub-array is about 10 microns or less. In some embodiments, the width of the sub-array is 10000 microns or more. In some embodiments, the width of the sub-array is about 10 microns to about 10000 microns. In some embodiments, the width of the sub-array is about 100 microns to about 5000 microns. In some embodiments, the width of the sub-array is about 1363 microns to about 1383 microns. In some embodiments, the width of the sub-array is about 1373 microns. In some embodiments, the 1 width of the sub-array is 1373 microns. In some embodiments, the length or width of the sub-array is 1373 microns or more. In some embodiments, the length or width of the sub-array is no less than 1373 microns. In some embodiments, the length or width of the sub-array may be greater than 1373 microns.

In some embodiments, the capture site layout, or equivalently the well layout, is defined as the number of wells aligned horizontally and the number of wells aligned vertically. In some embodiments, the wells are arranged in a square packed (e.g., linear) pattern. In some embodiments, the wells are arranged in a hexagonal packed (e.g., diamond packed) pattern. As shown in FIG. 39C the capture site layout, or equivalently the well layout, of a sub-array may be comprised of 108 rows of capture sites by 94 columns of capture sites. In some embodiments, the sub-array may comprise at least one row. In some embodiments, the sub-array may comprise at least 10 rows. In some embodiments, the sub-array may comprise at least 100, 150, 200, or 500 rows. In some embodiments, the sub-array may comprise 500 or more rows. In some embodiments, the sub-array may comprise at least one column. In some embodiments, the sub-array may comprise at least 10 columns. In some embodiments, the sub-array may comprise at least 100, 150, 200, or 500 columns. In some embodiments, the sub-array may comprise 500 or more columns.

### Substrate

Described herein are various devices 200 comprising substrates 203. In some embodiments, the substrate may comprise silicon, semiconductors, silicon, silicon dioxide, quartz, doped silicon, glass, plastics, ceramics, and metals or any combination thereof.

In some embodiments, the substrate may comprise silicon-on-insulator (SOI) substrates. In some embodiments, the insulator may comprise silicon oxide. In some embodiments, the insulator layer may be referred to as a buried oxide (BOX) layer. In some embodiments, the SOI substrates may comprise a device layer, an insulator layer, a handle layer, or a combination thereof.

In some embodiments, the handle layer thickness is about 300 microns to about 950 microns. In some embodiments, the handle layer thickness is at least about 300 microns.

In some embodiments, the device layer thickness is about 5 microns to about 100 microns. In some embodiments, the device layer thickness is at least about 5 microns. In some embodiments, the device layer thickness is about 30 microns.

In some embodiments, the substrate is a crystalline silicon wafer. In some embodiments, the crystalline silicon wafer comprises a crystal orientation. In some embodiments, the crystal orientation comprises of 100, 111, or 110. In some embodiments, substrate materials comprise of semiconductors, silicon, doped silicon, glass, plastics, ceramics, and metals or any combination thereof. In some instances, silicon may be doped. In some embodiments, substrate materials may be polycrystalline or amorphous. In some embodiments, substrate material size and dimensions determine the ease of manufacturing.

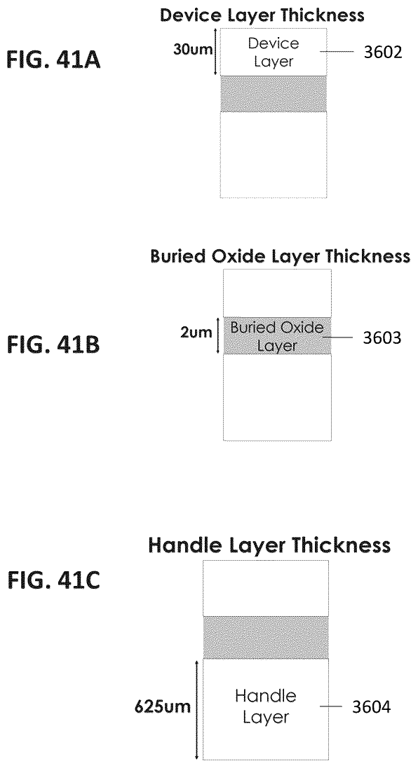

In some embodiments, the device layer thickness may be 30 micrometers (microns) thick as depicted in FIG. 41A. In some embodiments, the device layer thickness is 30+/−0.5 microns. In some embodiments, the device layer thickness is about 29.5 to about 30.5 microns. In some embodiments, the device layer thickness is 29.5 microns or less. In some embodiments, the device layer thickness is 30.5 microns or more. In some embodiments, the device layer thickness is about 30 microns. In some embodiments, the device layer thickness is 30 microns. In some embodiments, the device layer thickness refers to the distance from the plane of the well's top surface (e.g., device surface 202) to the top plane of the buried oxide layer. In some embodiments, the port channel length (e.g., aspiration via channel length) through the device layer controls the fluid transport and resulting pressure differential during device operation.

In some examples, the device layer thickness may be at most about 900, 800, 700, 600, 500, 400, 300, 200, 100 microns or less. In some examples, the device layer thickness may be at most about 100, 90, 80, 70, 65, 60, 55, 50, 45, 40, 35, 30, 25, 20, 19, 18, 17, 16 15, 14, 13, 12, 11, 10, 9, 8, 7, 6, 5, 4, 3, 2, 1 micron or less. In some examples, the device layer thickness may be at least about 0.5, 1, 2, 3, 4, 5, 6, 7, 8, 9, 10, 11, 12, 13, 14, 15, 16, 17, 18, 19, 20, 30, 40, 50, 60, 70, 80, 90, 100 or more.

As shown in FIG. 41B, in some examples, the BOX layer thickness may be 2 microns thick. In some embodiments, the BOX layer thickness may be 2+/−0.1 microns. In some embodiments, the BOX layer thickness may be about 1.9 microns to about 2.1 microns. In some embodiments, the BOX layer thickness may be about 1.9 microns or less. In some embodiments, the BOX layer thickness may be about 2.1 microns or more. In some embodiments, the BOX layer thickness refers to the distance from top surface of the BOX layer to the bottom surface of the BOX layer.

In some cases, the BOX layer thickness may be at most about 200, 150, 100, 90, 80, 70, 60, 50, 40, 30, 20, 10, 9, 8, 7, 6, 5, 4, 3, 2, 1, 0.9, 0.8, 0.7, 0.6, 0.5, 0.4 microns or less. In some examples, the BOX layer thickness may be at least about 0.5, 0.6, 0.7, 0.8, 0.9, 1, 1.5, 2, 2.5, 3, 3.5, 4, 5, 6, 7, 8, 9, 10, 20, 30, 40, 50, 60, 70, 80, 90, 100, 150 microns or more.

As shown in FIG. 41C, the handle layer of the wafer may be 610 microns thick. In some embodiments, the handle layer thickness may be referred to as the distance from the bottom surface of the buried oxide layer to the backside surface of the wafer. In some embodiments, the handle layer thickness may be 610 microns or less. In some embodiments, the handle layer thickness may be about 610 microns or more. In some embodiments, the handle layer thickness may be 610+/−20 microns. In some embodiments, the handle layer thickness may be about 500 microns to about 700 microns. In some embodiments, the handle layer thickness may be about 620 microns to about 630 microns. In some embodiments, the handle layer thickness may be about 610 microns. In some embodiments, the handle layer thickness may be 610 microns.

In some examples, the handle layer of the wafter may be at least about 10, 20, 30, 40, 50, 60, 70, 80, 90, 100, 150, 200, 300, 400, 500, 600, 700, 800, 900 microns or thicker. In some examples, the handle layer of the wafter may be at most about 900, 800, 700, 600, 500, 400, 300, 200, 100, 90, 80, 70, 60, 50, 40 30, 20 microns or thinner.

### Capture Sites

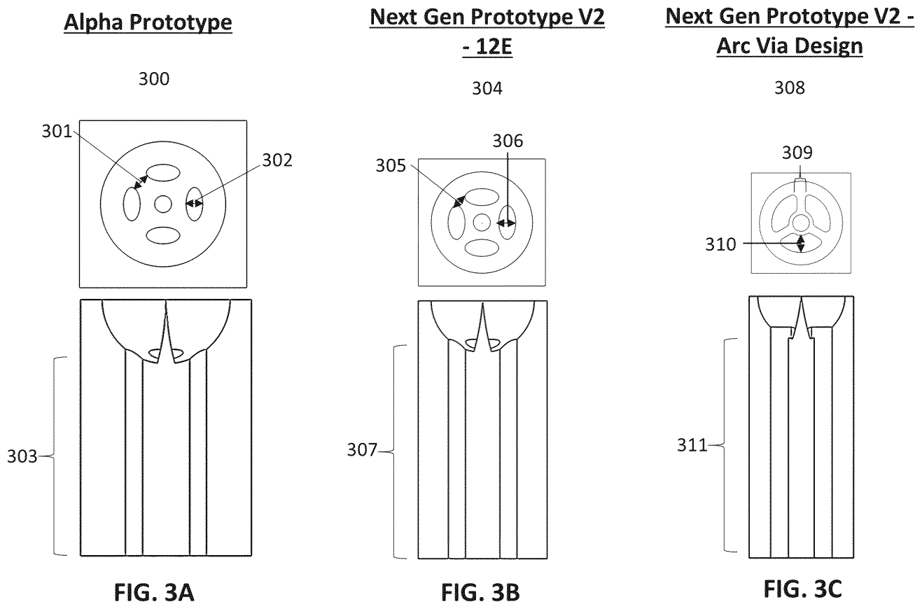

Described herein are various devices 200, comprising: a substrate 203 comprising: a surface 202; and a capture site 205, the capture site comprising: a well 206 having a well boundary shape (e.g., circle) 207 at the surface 202 and having a well depth 212 and a well shape diameter 211, the well configured to support a cell; and a projection/needle 209 extending from the bottom of the well toward the surface of the substrate, the projection/needle comprising a projection/needle length 210, and wherein the projection/needle length and the well depth comprise a projection length-well depth ratio as shown in FIG. 2. In some embodiments, the capture site comprises a port 208. In some embodiments, the capture site comprises a plurality of ports, as shown in FIGS. 3A-3C. In some embodiments, the capture site is configured for the capture of, perforation (e.g., poration) of and release of a cell. In some embodiments, the well boundary shape comprises a well boundary perimeter.

In some embodiments, the device 200 does not comprise an electrode. In some embodiments, the device 200 does not comprise an electrode configured for capture or release of the cell. In some embodiments, the device does not comprise an electrode configured for electrophoresis. In some embodiments, the device does not comprise an electrode configured for electrophoretic capture and release of cells.

### Well

In some embodiments, the capture site 205 comprises a well 206. In some embodiments, the well is configured for the capture of a cell as shown in FIGS. 1A-1F. In some embodiments, the well boundary shape 207 comprises a circle, ellipse, square, rectangle, triangle, or a clover leaf shape. In some embodiments, the clover leaf well perimeter shape is the resultant well perimeter shape produced by the well and aspiration via etching processes. In some embodiments, the well boundary shape comprises a well boundary shape area. In some embodiments, the well boundary shape comprises a circle. In some embodiments, the well boundary shape comprises a well diameter. In some embodiments, the well boundary shape comprises a perimeter. In some embodiments, the well boundary shape diameter is between about 100 microns and about 1 micron. In some embodiments, the well boundary shape diameter is about 10 microns.

In some examples, the well boundary shape dimeter may be at least about 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 200, 210, 220, 230, 240, 250, 260, 270, 280, 300, 400, 500, microns or larger. In some examples, the well boundary shape diameter may be at most about 800, 700, 600, 500, 400, 300, 200, 100, 90, 80, 70, 60, 50, 40, 30, 20, 10, 9, 8, 7, 6, 5, 4, 3, 2, 1 or smaller.

In some embodiments, the well depth 212 is about 50 microns to about 0.5 microns. In some embodiments, the well depth is about 7.5 microns to about 5 microns. In some embodiments, the well depth is about 5 microns.

In some examples the well depth may be at most about 500, 400, 300, 200, 100, 50, 40, 30, 20, 10, 9, 8, 7, 6, 5, 4, 3, 2, 1, 0.5, 0.4, 0.3, 0.2, or less. In some examples, the well depth may be at least about 0.1, 0.2, 0.3, 0.4, 0.5, 0.6, 0.7, 0.8, 0.9, 1, 2, 3, 4, 5, 6, 7, 8, 9, 10, 11, 15, 20, 30, 40, 50, 60, 70, 80, 90, 100, 200, 300, 400 microns or larger. In some examples, the well depth may be in the range between 0.5 micron to 10 microns, between 0.5 micron to 8 microns, between 0.5 micron to 7 microns, or between 0.5 micron to 5 microns.

In some embodiments, the well perimeter is about 1.5 microns to about 160 microns. In some examples, the well perimeter may be at least about 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 17, 19, 20, 21, 22, 23, 24, 25, 30, 35, 40, 50, 60, 70, 80, 90, 100, 110, 120, 130, 140, 150, 160 microns or larger. In some examples, the perimeter may be at most about 400, 300, 200, 190, 180, 170, 160, 150, 140, 130, 120, 110, 100, 90, 80, 70, 60, 50, 40, 30, 20, 10, 9, 8, 7, 6, 5, 4, 3, 2, 1, 0.9, 0.8, 0.7, 0.6, 0.5, or less.

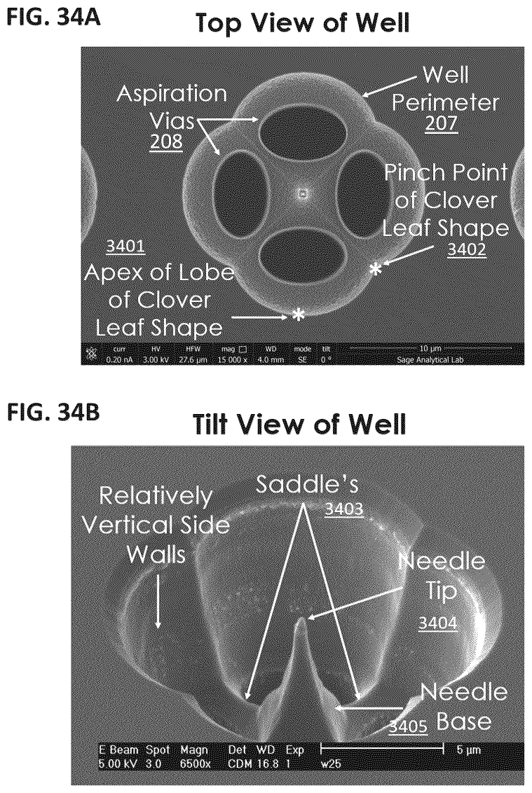

In some embodiments, as shown in FIGS. 34A and 34B, a well 206 comprises a clover leaf shaped well boundary perimeter. In some embodiments, the clover leaf shaped well boundary comprises an apex of the lobe 3401 and a pinch point 3402 as depicted in FIG. 34A. In such embodiments, the capture site also comprises sloped side walls (e.g., relatively vertical side walls), saddles, a projection/needle, and a projection/needle base. In some embodiments, contained within the well are aspiration vias as shown in FIG. 34A. In some embodiments, as shown in FIG. 34B, the well of the capture site may comprise relatively vertical side walls and the well may contain within itself, saddles 3403, a projection/needle tip (e.g., needle tip) 3404, and projection/needle base (e.g., needle base) 3405.

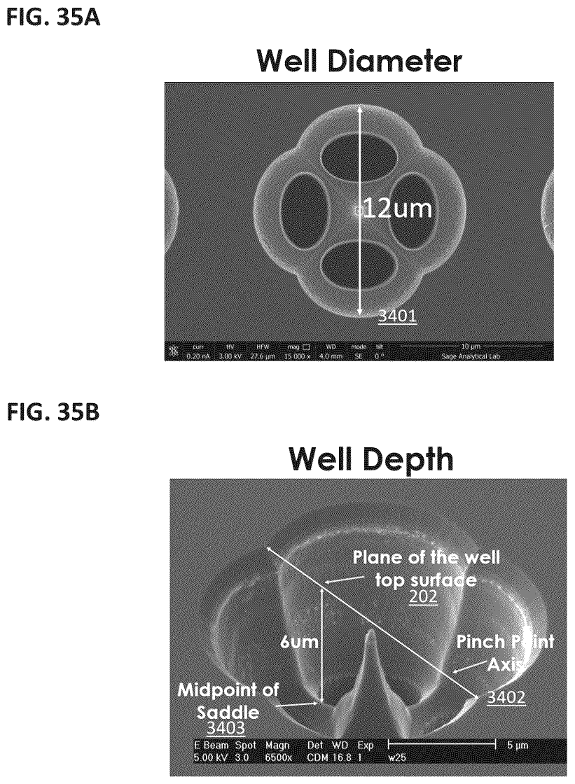

In some embodiments, as shown in FIG. 35A the well diameter as measured from one apex of lobe of clover leaf shape 3401 to the opposite apex lobe may be 12 microns. In some embodiments, the well diameter may be 12+/−0.5 microns as measured from one apex of the lobe of the clover leaf shaped well boundary perimeter (e.g., well perimeter) to the opposite lobe's apex. In some embodiments, the well diameter from the apex of one lobe of the clover leaf to the apex of the opposing lobe is symmetric in the vertical and horizontal directions. As shown in FIG. 35B, the well depth of the capture site may be 6 microns. In some embodiments, the well depth of the capture site may be 6 microns or more. In some embodiments, the well depth of the capture site may be 6 microns or less. In some embodiments, the well depth is measured from the midpoint of the saddle to top surface of the device (e.g., plane of the well top surface) 202. In some embodiments, the top surface of the device is identified by the axis line running between two pinch points of the clover leaf shaped well boundary, as shown in FIG. 35B. In some embodiments, the well depth is defined as the distance from the plane of the well top surface, down to the midpoint of a saddle. In some embodiments, the plane of the top surface of the well extends from the pinch point 3402 of the clover leaf to the pinch point of the opposing clover leaf.

In some examples, the well diameter may be at most about 200, 100, 90, 80, 70, 60, 50, 40, 30, 20, 19, 18, 17, 16, 15, 14, 13, 12, 11, 10, 9, 8, 7, 6, 5, 4, 3, 2 microns or less. In some examples, the well diameter may be at least about 1, 2, 3, 4, 5, 6, 7, 8, 9, 10, 11, 12, 13, 14, 15, 16, 17, 18, 19, 20, 30, 40, 50, 60, 70, 80, 90, 100, 200 or more.

### Projection/Needle

In some embodiments, the capture site 205 comprises a projection (e.g., needle, needle-like projection, static needle, needle protrusion, or protrusion) 209. In some embodiments, the capture site comprises one needle/projection. In some embodiments, the capture site comprises a plurality of needles/projections. In some embodiments, the plurality of needles/projections comprises 2 projections/needles to 100 projections/needles. In some embodiments, the projection/needle is configured to perforate the cell, forming a perforation (e.g., pore) in the cell membrane and/or cell wall as shown in FIGS. 1A-1F. In some examples, a projection may comprise or be a needle, a static needle, or a needle-like projection. The terms projection and needle may be used interchangeably. The needle may be a static needle.

In some examples, the capture site may comprise a plurality of projections/needles. The plurality of projections/needles may comprise at least 1, 2, 3, 4, 5, 6, 7, 8, 9, 10, 20, 30, 40, 50, 60, 70, 80, 90, 100, 150, 200, or more projections/needles. In some examples, the plurality of projections/needles may comprise at most about 200, 150, 100, 90, 80, 70, 60, 50, 40, 30, 20, 10, 9, 8, 7, 6, 5, 4, 3 projections/needles or less.

In some embodiments, the projection/needle 209 has a projection/needle length 210. In some embodiments, the projection/needle length may be about 50 microns to about 0.5 microns. In some embodiments, the projection/needle length is about 6 microns to about 2 microns. In some embodiments, the projection/needle length is about 3.5 microns. In some embodiments, the projection/needle is referred to as a needle or a protrusion.

In some examples the projection/needle length may be from at least about 0.5 microns to about 100 microns. In some examples, the projection/needle length may be at least 0.5, 0.6, 0.7, 0.8, 0.1, 1, 2, 3, 4, 5, 6, 7, 8, 9, 10, 11, 12, 13, 14, 15, 16, 17, 18, 19, 20, 30, 40, 50, 60 microns or longer. In some examples, the projection/needle length may be at most about 100, 90, 80, 70, 60, 50, 40, 30, 20, 10, 9, 8, 7, 6, 5, 4, 3, 2, 1, 0.9, 0.8, 0.7, 0.6, 0.5 or smaller.

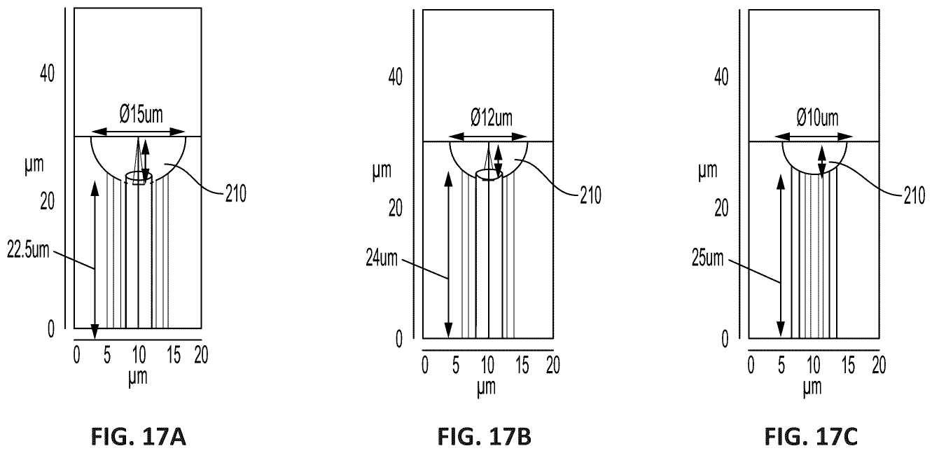

In some embodiments, the projection (e.g., needle, static needle, needle-like projection or protrusion) comprises a projection tip (e.g., needle tip or protrusion tip). In some embodiments, the projection tip comprises the top of the projection. In some embodiments, as depicted in FIGS. 17A-17C, the projection length is configured for flow focusing above the projection tip.

The terms needle, static needle, needle-like projection, and projection may be used interchangeably.

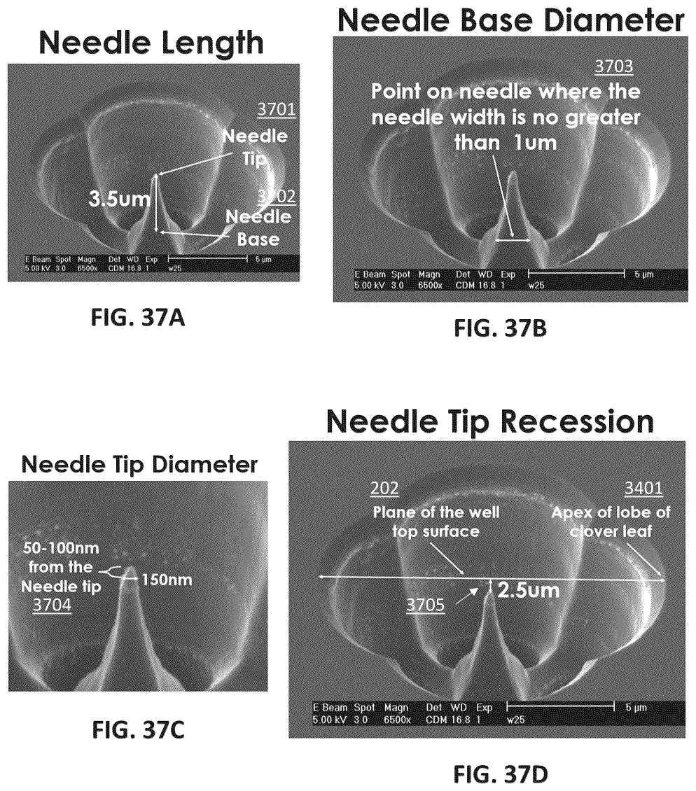

In some embodiments, the projection (e.g., needle or protrusion) comprises a projection base. In some embodiments, the projection/needle base refers to a horizontal cross section, or width, of the projection, as depicted in FIG. 37B, where the projection is 1 micron. In some embodiments, the projection base is equal to about 1 micron. In some embodiments, the projection base (e.g., needle base or protrusion base) refers position along the vertical axis of the projection, where the projection width (e.g., diameter) becomes no greater than 1 micron, as the projection's vertical axis is traced, starting from the projection tip and downward, toward the bottom of the well. In some embodiments, the projection base refers to a position between the projection tip down to the point on the lower distal portion of the projection where there is no projection width greater than 1 micron.

In some embodiments, the projection length may be engineered to accommodate a target cell to be captured. In some embodiments, the projection length is configured for a projection length to cell diameter ratio. In some embodiments, the projection length to cell diameter ratio may be determined by the diameter of the cell chosen, specifically to enter the nucleus but, in some cases, not go through the entire cell.

In some embodiments, the projection/needle comprises a projection tip. In some embodiments, the projection tip diameter is measured at a distance of 50 nanometers from the absolute tip (e.g., projection tip end). In some embodiments, a projection comprises a projection tip diameter to projection base diameter ratio. In some embodiments, the projection tip diameter is about 100 nanometers. In some embodiments, the projection base diameter is about 2000 nanometers.

In some embodiments, the projection tip diameter to projection base diameter is about 100:2000. In some embodiments, the projection tip diameter to projection base diameter is about 1:20. In some embodiments, the projection tip diameter to projection base diameter is about at most 1:20. In some embodiments, the projection tip diameter to projection base diameter is about at least 1:20.

In some embodiments, the capture site, or the well, comprises a projection (e.g., needle, static needle, needle-like projection, needle protrusion, or protrusion). In some embodiments, the projection (e.g., needle or protrusion) is centered at the bottom of the well and projects upward.

In some embodiments, the projection comprises a projection length. In some embodiments, the projection length is 1 micron or less. In some embodiments, the projection length is 10 microns or more. In some embodiments, the projection length is about 2 microns to about 5 microns. In some embodiments, the projection length is 3 microns to about 4 microns. In some embodiments, the projection length is about 3.5 microns. In some embodiments, the projection length is 3.5 microns. In some embodiments, the projection length is 3.5+/−0.5 microns. In some embodiments, the projection (e.g., needle) is the protrusion created by the etching process. In some embodiments, the projection comprises the shape of a pointed pillar or conical needle.

In some embodiments, the projection/needle comprises a projection/needle base diameter. In some embodiments, the projection base diameter is 0.1 microns or less. In some embodiments, the projection base diameter is 10 microns or more. In some embodiments, the projection base diameter is 0.1 microns to 10 microns. In some embodiments, the projection base diameter is 0.5 microns to 1.5 microns. In some embodiments, the projection base diameter (needle base diameter), is 1+/−0.5 microns at the location shown in FIG. 37B. The projection base (e.g., needle base) is defined as position along the projection height axis, where the width is no greater than 1 micron, as depicted in FIG. 37B. In some embodiments, the projection base is as close to the bottom of the well as possible.

In some examples, the projection/needle base diameter may be at most about 10, 9, 8, 7, 6, 5, 4, 3, 2, 1, diameter or less. In some examples, the projection base diameter may be at least about 0.1, 0.2, 0.3, 0.4, 0.5, 0.6, 0.7, 0.8, 0.9, 1, 2, 3, 4, 5, 6, 7, 8, 9 microns or more.

In some embodiments, the projection tip (e.g., needle tip) is defined as the top of the projection (e.g., needle, needle protrusion, or protrusion). The projection tip diameter (e.g., needle tip diameter) is measured from a point 50-100 nanometers downward from the tip of the projection. In some embodiments, the projection tip diameter is about 10 nanometers or less. In some embodiments the projection tip diameter is about 500 nanometers or more. In some embodiments, the projection tip diameter is about 10 nanometers to about 500 nanometers. In some embodiments, the projection tip diameter is about 100 nanometers to about 200 nanometers. In some embodiments, the projection tip diameter is about 150 nanometers. In some embodiments, the projection tip diameter is 150 nanometers as shown in FIG. 37C. In some embodiments, the verifiable acceptance criteria is for the projection tip diameter is 150+/−50 nanometers.

In some embodiments, the projection/needle tip recession (e.g., needle tip recession) is defined as the distance from the plane of the well top surface (e.g., device top surface 202) down to the tip of the needle. As shown in FIG. 37D, the plane of the top surface extends from the apex of one lobe of the clover leaf to the apex of the opposing lobe. In some embodiments, the projection tip recession is 1 micron or less. In some embodiments, the projection tip recession is 1 micron or more. In some embodiments, the projection tip recession is about 1 to about 10 microns. In some embodiments, the projection tip recession is about 2.5 microns to about 3 microns (e.g., 2.5+0.5, −0 microns). In some embodiments, the projection tip recession is about 2.5 microns. In some embodiments, the projection tip recession is 2.5 microns as depicted in FIG. 37D.

In some embodiments, the projection/needle 209 does not move relative to the device 200. In some embodiments, the projection 209 does not move relative to the surface 202 of the device 200. In some embodiments, the device does not comprise an actuator configured for moving the projection.

### Projection/needle Length—Well Depth Ratio

Described herein are various devices 200, comprising: a substrate 203 comprising: a surface 202; and a capture site 205, the capture site comprising: a well 206 having a well boundary shape 207 at the surface and having a well depth 212, the well configured to support a cell; and a projection 209 extending from the bottom of the well toward the surface of the substrate, the projection comprising a projection length 210, and wherein the projection/needle length and the well depth comprise a projection length-well depth ratio. In some embodiments, the projection length-well depth ratio is about 10% to about 100%. In some embodiments, the projection length-well depth ratio is from 40% to about 60%. In some embodiments, the projection length-well depth ratio is about 50%. The terms needle and projections may be used interchangeably throughout the disclosure.

In some examples, the projection/needle length-well ratio may be at most about 90%, 80%, 70%, 60%, 50%, 40%, 30%, 20%, 10%, 5%, or less. In some examples, the projection length-well ration may be at least about 5%, 10%, 20%, 30%, 40%, 50%, 60%, 70%, 80%, or larger.

The projection length-well ratio may in some cases be an important parameter for achieving the single cell capturing/encapsulation. Therefore, optimizing this ratio may aid in optimizing the methods performed using the devices of the present disclosure. This ratio may be designed as intended based on the application, and fabricated. The fabrication techniques detailed elsewhere herein may allow for achieving the desired projection length-well ratio. The terms needle and projections may be used interchangeably throughout the disclosure.

### Ports

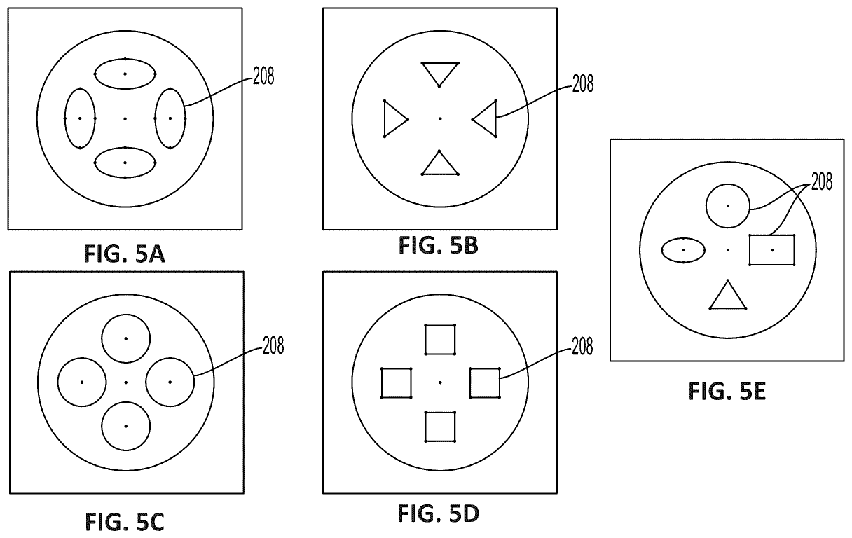

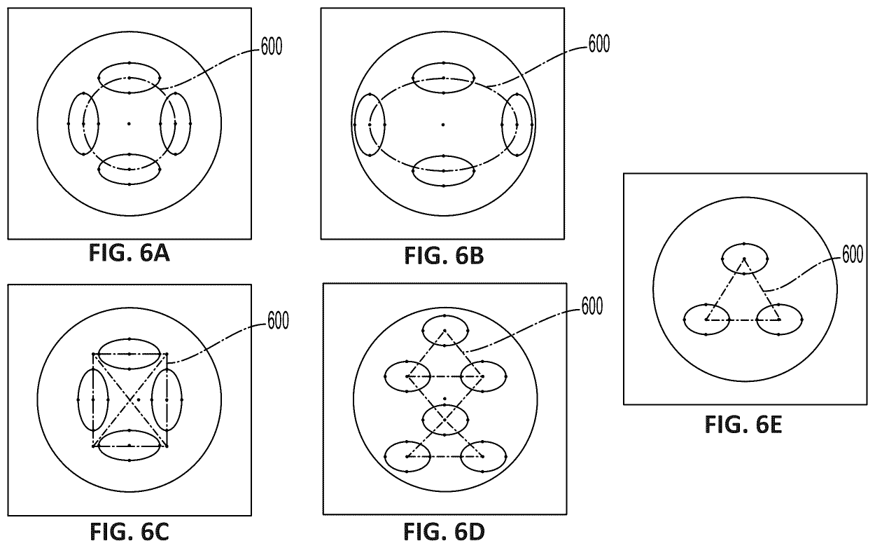

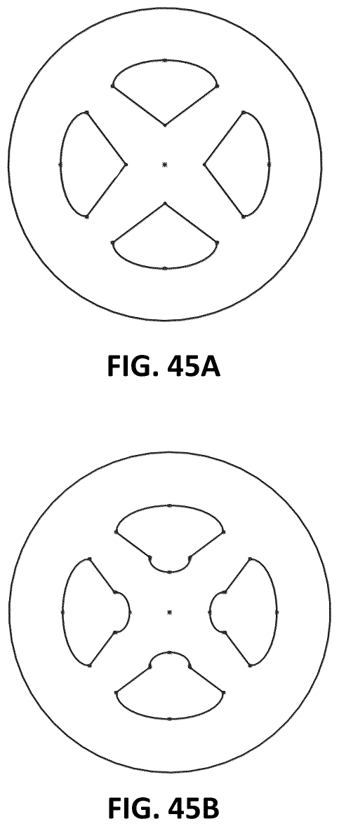

In some embodiments, capture site 205 comprises a port 208. In some embodiments, the port comprises an aspiration via. In some embodiments, the aspiration via is fluidly connected to and/or within a system, the system comprising a pump as shown in FIG. 9A. In some embodiments, the capture site is configured for the capture of, perforation of, and release of, a cell, as shown in FIGS. 1A-1F. In some embodiments, the port comprises a port boundary shape. In some embodiments, the port boundary shape comprises a circle, an ellipse, a square, a rectangle, a triangle, an arc via, or a partial arc via (e.g., arc) as depicted in FIGS. 5A-5E. In some embodiments, the port boundary shape comprises an irregular shape. FIGS. 45A and 45B depict ports comprising port boundary shapes in accordance with some embodiments. In some embodiments, the port boundary shape is configured to affect (e.g., manipulate) the final shape of the projection after the etching process. In some embodiments, the port boundary shape is configured to pinch off the projection from the oxide layer during fabrication of the projection. In some embodiments, the port boundary shape is configured to prevent merging port (e.g., aspiration via) channels during fabrication of the projection. In some embodiments, the port comprises a port boundary shape area. In some embodiments, the port boundary shape comprises a long axis. In some embodiments, the port comprises a plurality of ports. In some embodiments, the plurality of ports comprises about 1 port to about 100 ports. In some embodiments, the port arrangement 600 comprises of circular, elliptical, square, rectangular, triangular, diamond, hexagonal, or any combination thereof, as depicted in FIGS. 6A-6E. In some embodiments, the port comprises a lumen. In some embodiments, the projection 209 comprises the lumen. In some embodiments, the plurality of ports comprises a lumen and/or an aspiration via. In some embodiments, the capture site comprises a plurality of ports. In some embodiments, the port boundary shape area comprises a port boundary shape area about 1 micron squared to about 100 microns squared. In some embodiments, the aspiration via arrangement and shape are configured for uniformity of velocity magnitude across the channel length, inside and above the well.

In some embodiments, the port comprises a port channel length (e.g., aspiration via channel length) as depicted in FIGS. 3A-3C, wherein the port channel lengths 303, 307 and 311 are shown in FIGS. 3A, 3B, and 3C, respectively. In some embodiments the port channel length may be about 5 microns to about 100 microns. In some embodiments, the port channel length is greater than 3 microns. In some embodiments, the port channel length is greater than 5 microns. In some embodiments, the port length is greater than 10 microns. In some embodiments, the port channel length is greater than 20 microns. In some embodiments, the port length is 22.5 microns as depicted in FIG. 17A. In some embodiments, the port channel length is 24 microns as depicted in FIG. 17B. In some embodiments, the port length is 25 microns as depicted in FIG. 17C.

In some embodiments, a channel, comprising an elliptically shaped opening, that is formed in the device layer and travels down to region of the wafer that contains, or did contain, the buried oxide layer, is referred to as the aspiration via channel.

In some embodiments the capture site comprises a saddle 3403 depicted in FIG. 34B. In some embodiments, the saddle may comprise the resultant ridge line located in between adjacent ports (e.g., aspiration vias) produced by the well etching process.

In some embodiments, the well of the capture site comprises side walls. In some embodiments, the side walls of the well, located on the perimeter of the aspiration vias and rising roughly vertically up to the chip surface, that are produced by the well etching process as depicted in FIG. 34B.

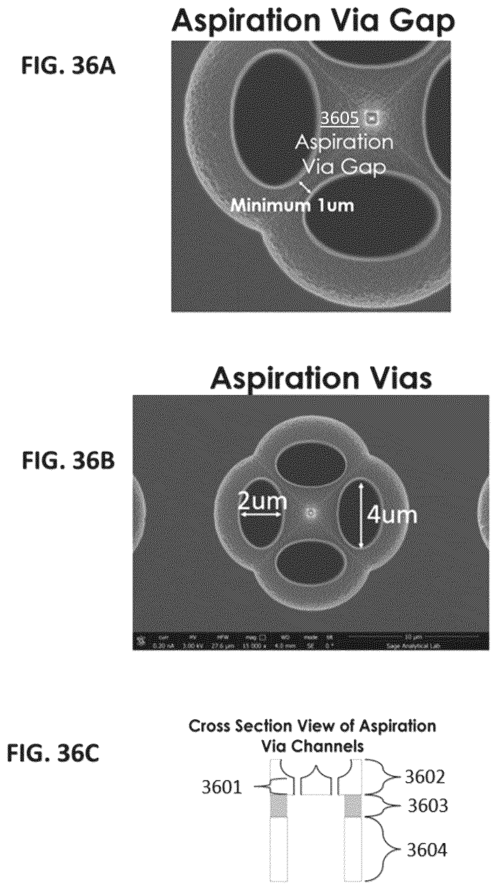

In some embodiments, a gap may be present between the boundary perimeters of two ports (e.g., aspiration vias) as depicted in FIG. 36A. In such embodiments, the gap between two aspiration via boundary perimeters is referred to as the aspiration via gap. In some embodiments, the gap refers to the closest distance between adjacent aspiration vias. In some embodiments, the aspiration via gap (e.g., port gap) is less than 1 micron. In some embodiments, the aspiration via gap is about 1 micron. In some embodiments, the aspiration via gap is greater than 1 micron. In some embodiments, the aspiration via gap is 0.8 microns. In some embodiments, the aspiration via gap is 1.2 microns. In some embodiments, the aspiration via gap is 0.8 microns to 1.2 microns. In some embodiments, the aspiration via gap is 1+/−0.2 microns.

In some embodiments, an elliptical port size (e.g., elliptical aspiration via size) may be defined by the short (e.g., width) axis and long (e.g., length) axis of the elliptical port boundary. In some embodiments, the long axis is 4+/−0.2 microns and the short axis is 2+/−0.2 microns. In some embodiments, the long axis of the port (e.g., aspiration via) is 4 microns and the short axis of the ports is 2 microns, as depicted in FIG. 36B. In some embodiments, there are four elliptical shaped ports located at bottom of each well distributed concentrically around the well center point. In some embodiments, the port is centered between the edge of the well to the center point of the well. In some embodiments, the port controls the fluid transport and resulting pressure differential during device operation. In some embodiments, as depicted in FIG. 36C the aspiration via channel length, extends from the bottom of a well to the top of the where the buried oxide layer resides prior to processing. FIG. 36C shows where the device layer, the buried oxide layer, and the handle layer of the wafer from which the device was made reside relative to where the device features reside. Such device features may include the well, ports, port channels, and projection. In some embodiments, the port channel length (e.g., aspiration via channel length) may be 24+/−0.5 microns. In some embodiments, the port channel length may be 23.5 microns to 24.5 microns. In some embodiments, the port channel length is about 23.5 microns to about 24.5 microns. In some embodiments, the port channel length may be 23.5 microns or less. In some embodiments, the port channel length may be 24.5 microns or more. In some embodiments, the capture site comprises four port channels. In some embodiments, as depicted in FIG. 36A-36B, the ports are located within the well. In some embodiments, the ports (e.g., aspiration vias) start at the bottom of the well and travel down completely through the device layer.

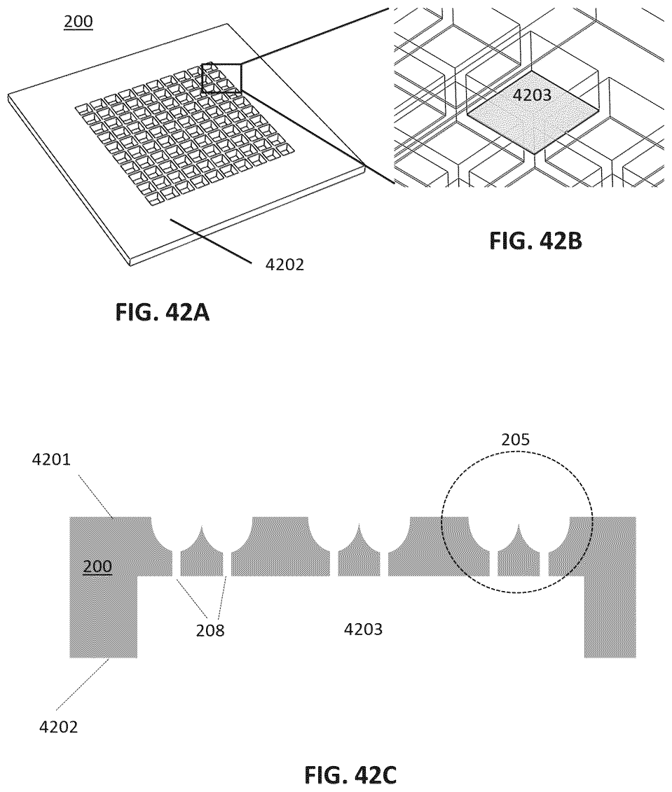

Described herein are various embodiments of a device comprising an aspiration port on the back side of the device. FIG. 42A shows a three-dimensional view of the back side 4202 of a device 200 having a 10 by 10 array of back side aspiration ports 4203. FIG. 42B shows a zoomed-in view of the area outlined in FIG. 42A. In some embodiments, the device 200 comprises a front side 4201 and a back side 4202 as depicted in FIG. 42C. In some embodiments, the front side of the device comprises an array of sub-arrays 201, where the array of sub-arrays comprises a plurality of capture sites 205. In some embodiments, the back side of the device comprises a back side aspiration port 4203 as depicted in FIG. 42C. In some embodiments, the back side aspiration port 4203 is fluidly connected to a plurality of ports (e.g., aspiration vias) 208 contained within each well of each capture site of the plurality of capture sites located on the front side of the device as depicted in FIG. 42C. In some embodiments, the back side aspiration port is fluidly connected to a plurality of aspiration vias contained within each well of each capture site of a sub-array of capture sites. In some embodiments, the back side aspiration port is fluidly connected a constant pressure system in accordance with some embodiments. In some embodiments, the back side aspiration port is fluidly connected a constant pressure system as described in Example 14 of the present disclosure.

### Ratios

In some embodiments, the port boundary shape area and well boundary shape area comprise a port boundary shape area to well boundary shape area ratio. In some embodiments, the port boundary shape area to well boundary shape area ratio is about 1:1.1 to about 1:10. In some embodiments, the port boundary shape area to well boundary shape area is about 25 square microns to about 176 square microns. In some embodiments, the port boundary shape area to well boundary shape area ratio is about 1:7. In some embodiments, the port boundary shape area to well boundary shape area ratio is about 25 square microns to about 78 square microns. In some embodiments, the port boundary shape area to well boundary shape area ratio is about 1:3.

In some embodiments, the port comprises an aspiration via. In some embodiments, the aspiration via comprises an aspiration via shape. In some embodiments, the aspiration via shape comprises an ellipse. In some embodiments, the capture site, comprises a plurality of elliptical shaped aspiration vias. In some embodiments, the capture site comprises four elliptically shaped aspiration vias as shown in FIGS. 2, 3A-3B, 5A, 5E, 6A-6E, 10A, 10B, 1A, 11B, 16A and 16B.

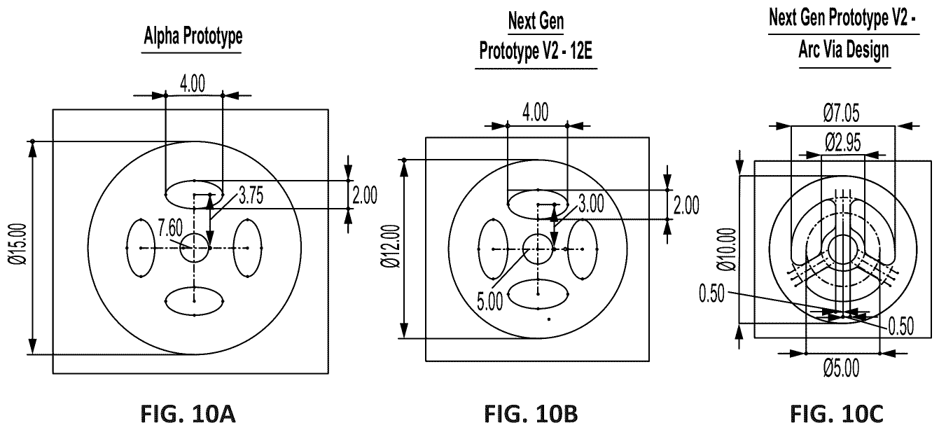

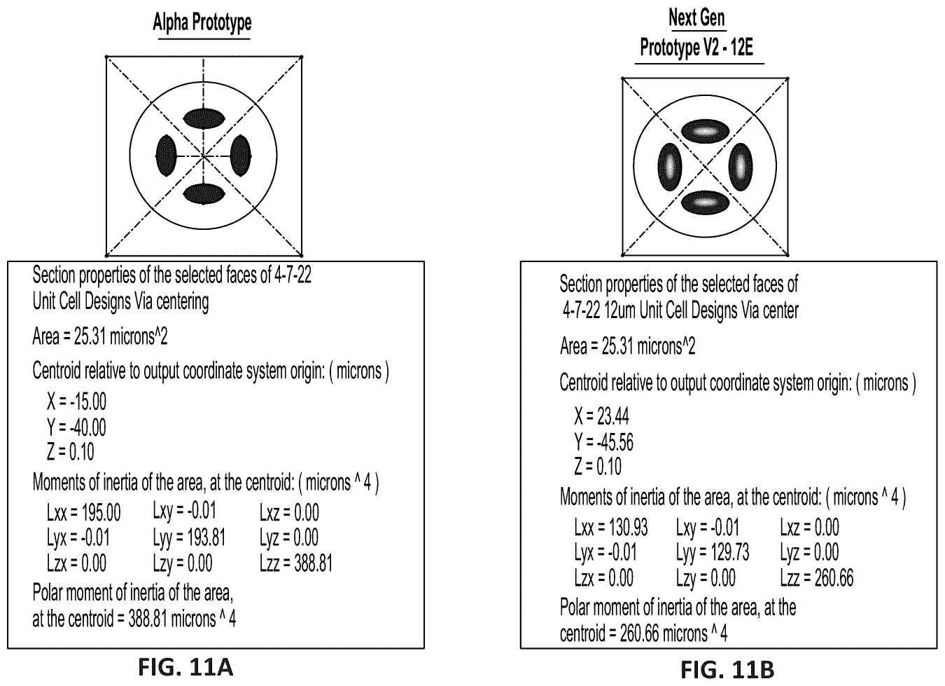

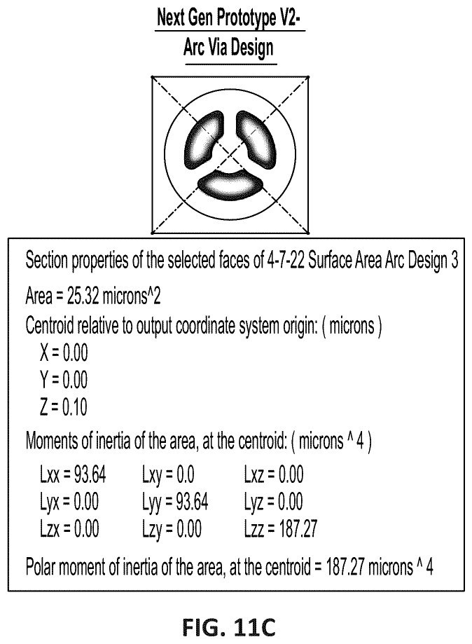

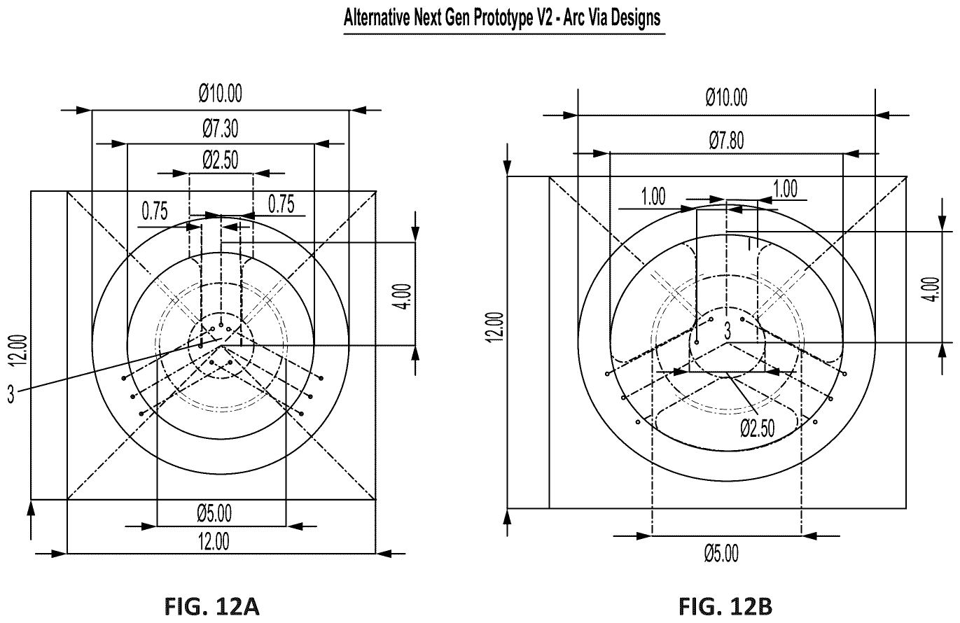

In some embodiments, the four aspiration vias as depicted in FIGS. 11A, comprise a total surface area of 25.31 square microns, as listed in FIG. 11B. In some embodiments, the capture site and elements of the capture site comprise spatial parameters. In some embodiments, the spatial parameters comprise a port boundary shape area. In some embodiments, the spatial parameters comprise a centroid relative to output coordinate system origin. In some embodiments, the spatial parameters comprise moments of inertia of the area, at the centroid. In some embodiments, the spatial parameters comprise polar moment of inertia of the area, at the centroid. In some embodiments, the spatial parameters comprise angles between the principal axes. In some embodiments, the spatial parameters comprise principal moments of inertia of the area, at the centroid. In some embodiments, the spatial parameters comprise moments of inertia of the area, at the output coordinate system. Spatial parameters and values of the capture site and elements of the capture site, including the aspiration vias, comprise the values are displayed in FIGS. 11A-11C in accordance with some embodiments. In some embodiments, the aspiration via comprises an aspiration via shape. In some embodiments, the aspiration via shape comprises an arc via. In some embodiments, the arc via comprises a shape as depicted in FIGS. 10C, 11C, 12A, 12B, 13A, and 13B. FIG. 12A shows a non-limiting example of an arc via-shaped aspiration via designed to be located within a 10-micron diameter well in accordance to some embodiments. In some embodiments, the arc-shaped aspiration via comprises segments that are not connected. In some embodiments, the arc via segments that are not connected are referred to as partial arc vias and/or arcs. In some embodiments, the capture site 205, comprises a unit cell. In some embodiments, the capture site, comprises a unit cell width. In some embodiments, the unit cell width is 12 microns as depicted in FIGS. 12A and 12B. In some embodiments, the capture site 205 comprises a minimum gap between the arc-shaped aspiration vias (e.g., arcs). In some embodiments, the minimum gap between arc-shaped aspiration vias is 1 micron. In some embodiments, the arc-shaped aspiration vias are centered on the well diameters as depicted in FIGS. 10-16. In some embodiments, an arc-shaped aspiration via comprises a thickness. In some embodiments, the thickness of the arc-shaped aspiration via may be adjusted to keep surface area equivalency between alternative aspiration via designs. In some embodiments, the arc-shaped aspiration via comprises corner fillets. In some embodiments, the arc-shaped aspiration via comprises 0.5 microns corner fillets.

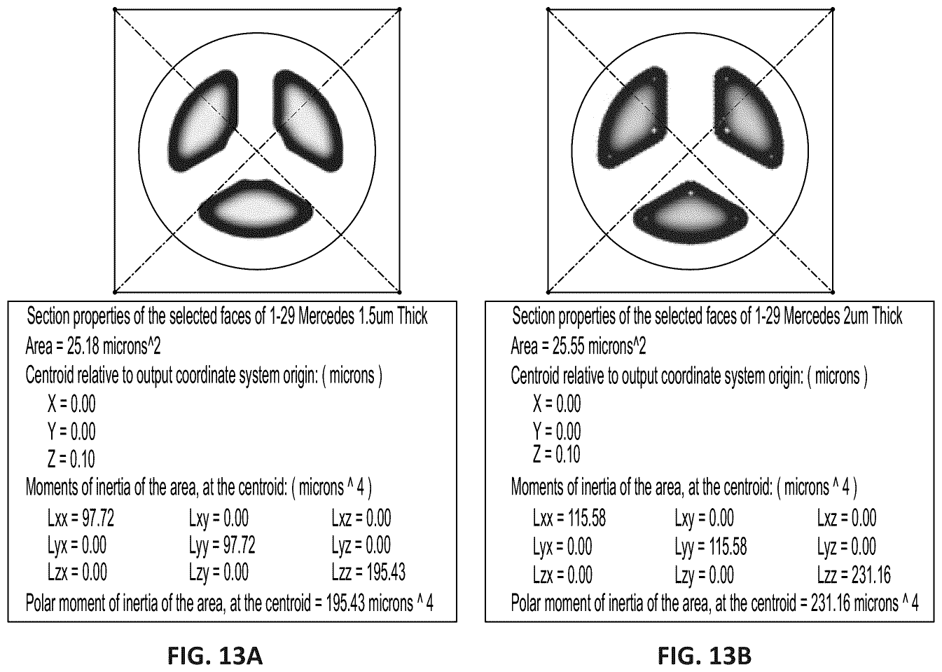

FIGS. 12A-12B show non-limiting examples of alternative Next Gen Prototype V2—Arc Via designs. In some embodiments, the minimum gap between arc-shaped aspiration vias is 1.5 microns as depicted in FIG. 12A. In some embodiments, the minimum gap between arc-shaped aspiration vias is 2 microns as depicted in FIG. 12B. In some embodiments, the minimum gap between arc-shaped aspiration vias is between about 100 nanometers and about 90 microns. In some embodiments, the total surface area of arc-shaped aspiration vias of a capture site is 25.18 microns squared or 25.55 microns squared as depicted in FIGS. 13A-13B, respectively.

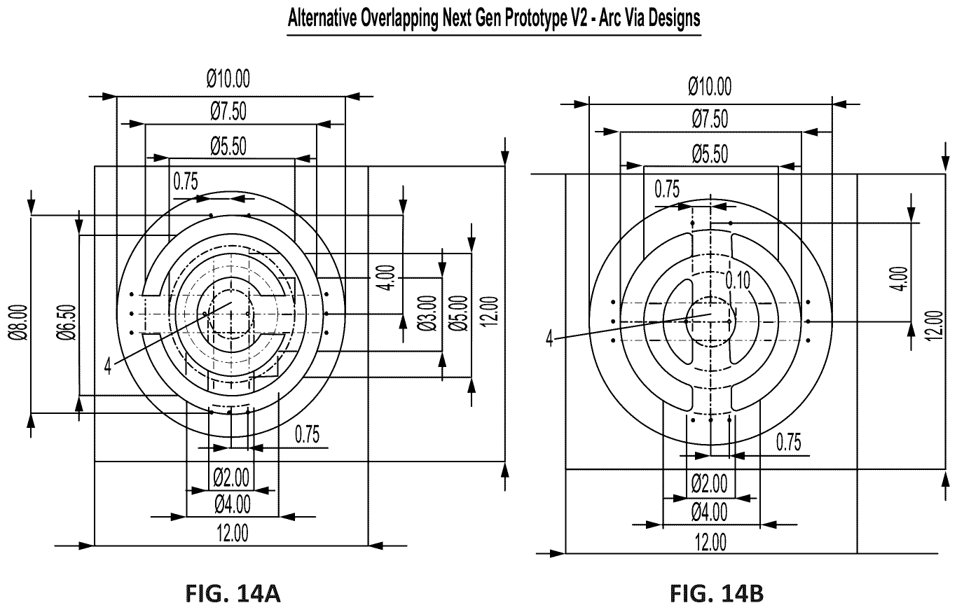

In some embodiments, the aspiration via shape may comprise an arc via. In some embodiments the arc shaped aspiration via may comprise a plurality of arc shaped vias, a plurality of partial arc via shaped aspiration vias (e.g., arc) or a combination there of. In some embodiments, the arc shaped aspiration via may comprise overlapping arc-shaped aspiration vias, as shown in FIGS. 14A and 14B. In some embodiments, the partial arc via is referred to as an arc. In such an embodiment, the well may comprise a diameter of 10 microns. In such an embodiment, the overlapping arc (e.g., partial arc shaped aspiration via) comprises a thickness of 1 micron as depicted in FIG. 14A. In some embodiments, the minimum gap between arcs is 1 micron, as depicted in FIG. 10C. In some embodiments, the arc comprises a thickness of 2.4 microns with a minimum gap of 1.5 microns, as depicted in FIG. 12A. In some embodiments, the arc comprises a thickness of 2.6 microns with a minimum gap of 2 microns, as depicted in FIG. 12B.

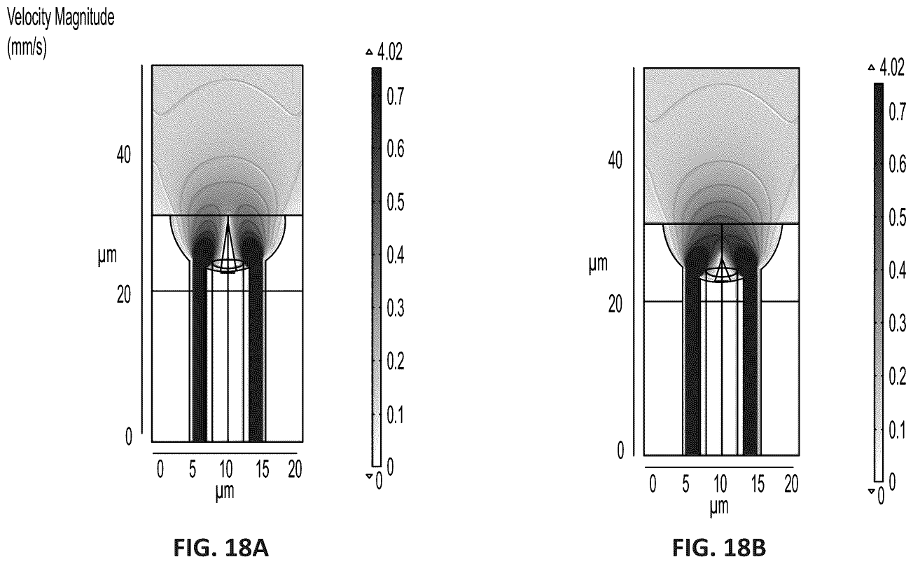

In some embodiments, as depicted in FIG. 13A, the arc-shaped vias are configured for uniformity of flow velocity magnitude across the well. In some embodiments, uniformity of flow velocity magnitude may be achieved at both the well 206 (e.g., capture site 205) and chip scale (e.g., chip comprising one or more array(s) of sub-arrays 200. At the well 206 scale the flow velocity magnitude profile may be uniform across each port (e.g., aspiration via) channel as depicted in FIG. 18-20. At the chip scale, the flow velocity magnitude is uniform above each well, across the chip surface as depicted in FIG. 18-20.

In some embodiments, a projection 210 length may be optimized for flow velocity magnitude of the fluid over the projection (e.g., needle) 209. In some embodiments, wherein the projection length is less than the well depth 212 (e.g., the projection tip is close in proximity the aspiration vias) the flow velocity magnitude of the fluid over the projection increases relative to a projection tip closer to the surface 202 (e.g., the top surface of the device 200). In some embodiments, wherein the projection length is closer to the upper surface and the projection tip is further in proximity to the aspiration vias 208, the flow velocity magnitude above the projection is reduced relative to some embodiments.

In some embodiments, the flow velocity magnitude into and/or out of the aspiration via is controlled by a pressure applied from the pump 902 as depicted in FIG. 9A. In some embodiments, the flow velocity magnitude into the aspiration via is actuated by applying a negative pressure 912 to the aspiration via channel. In some embodiments, the flow velocity magnitude out of the aspiration via is actuated by applying positive pressure 913 to the aspiration via channel. In some embodiments, the flow velocity magnitude of the fluid is simulated for various capture site configurations. In some embodiments, the capture site comprises a design as depicted in FIGS. 18A-18B. In some embodiments, as depicted in FIG. 18A, the projection length is about the same as the well depth, wherein the projection length to well depth ratio is about 1. In some embodiments, as depicted in FIG. 18B the projection length 210 to well depth ratio is much less than 1, wherein the projection tip is below the surface 202 (e.g., top surface of device) and closer to the aspiration vias 208, relative to the embodiment shown in FIG. 18A. In such embodiments, wherein the projection length is below the surface 202 (e.g., top surface of device) and closer to the aspiration vias 208, the flow velocity magnitude is higher and more uniform near the projection tip as shown in FIG. 18B, as compared to that shown in FIG. 18A. In some embodiments, the reduction in projection length 210 (e.g., shorter distance between the projection tip to the aspiration vias) improves the flow to be more focused over the projection tip.

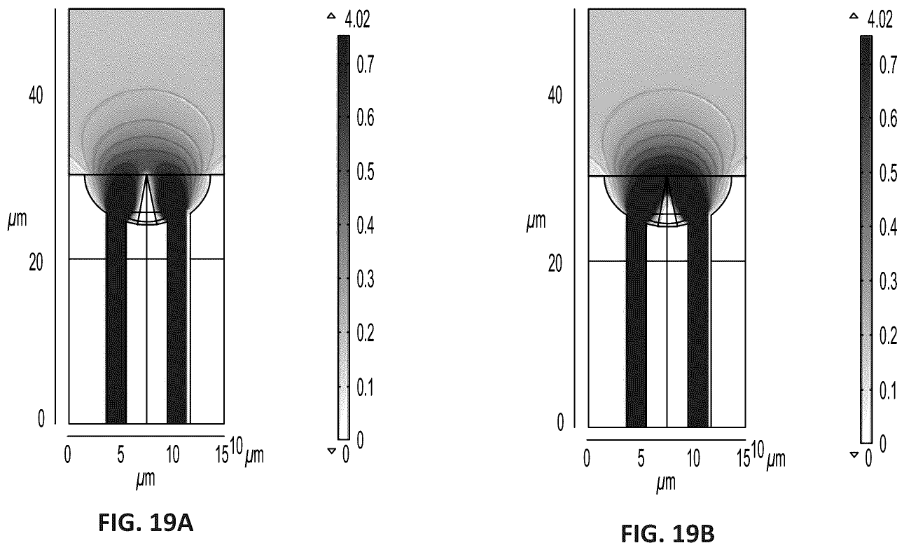

In some embodiments, the capture site comprises a design, wherein the well shape exhibits a 12-microns diameter, as depicted in FIG. 19A-19B. In some embodiments, as depicted in FIG. 19B the projection length 210 to well depth ratio is less than 1, wherein the projection tip is below the surface 202 (e.g., top surface of device) and closer to the aspiration vias 208, relative to the embodiment shown in FIG. 19A. In such embodiments, wherein the projection length is below the surface and closer to the aspiration vias, the flow velocity magnitude is higher and more uniform near the projection tip as shown in FIG. 19B.

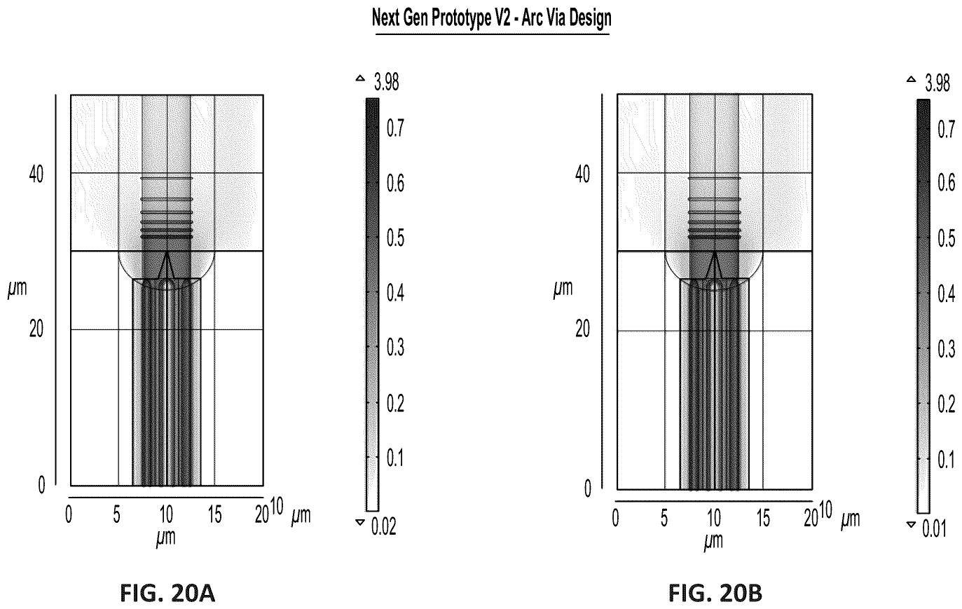

In some embodiments, the capture site comprises a Next Gen Prototype V2—Arc Via design, as depicted in FIGS. 20A and 20B. In some embodiments, as depicted in FIG. 20B the projection length 210 to well depth ratio is much less than 1, wherein the projection tip is below the surface 202 and closer to the aspiration vias 208, relative to the embodiment shown in FIG. 20A. In such embodiments, wherein the projection length is below the surface 202 and closer to the aspiration vias 208, the flow velocity magnitude is higher and more uniform near the projection tip as shown in FIG. 20B.

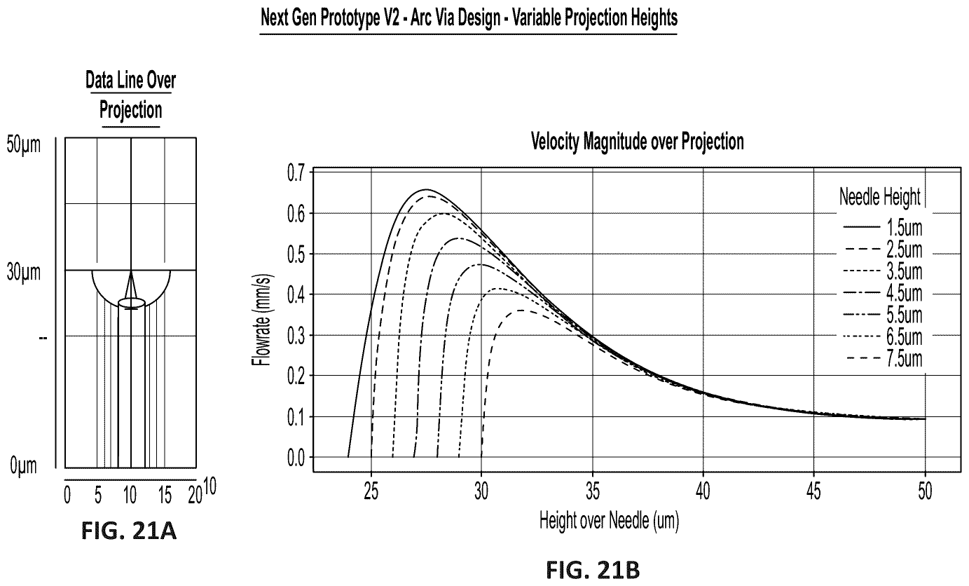

Provided herein are various embodiments, of capture sites 205, wherein the projection length 210 of the projection 209 of each embodiment may vary from other embodiments of the various embodiments. FIGS. 21A and 21B show, for various embodiments, that the velocity magnitude values above the projection increasing as projection lengths are decreased. FIG. 21A shows a vertical data line drawn from the projection tip to a height of 50 microns over a 0 point of the simulation.

In some embodiments, aspiration via comprise an arc shape, as depicted in FIGS. 11C, 12A-12B, and 14A-14B. In such an embodiment, the well may comprise a diameter of 10 microns. In such an embodiment, a first arc (e.g., partial arc shaped aspiration via) comprises a thickness of 0.75 microns and a second arc comprises a thickness of 1 micron. In some embodiments, the minimum gap between arcs is 1 micron. In some embodiments, the arc-shaped aspiration vias are configured for uniformity of fluid dynamics across the well.

In some embodiments, the well may comprise 1 to about 10 arc-shaped aspiration vias. In some embodiments, the well may comprise 1 to about 20 arcs (e.g., partial arc shaped aspiration via). In some embodiments, the arc-shaped aspiration via and/or arc comprises a thickness of about 0.5 microns to about 25 microns. In some embodiments, the minimum gap between arc-shaped aspiration vias and/or arcs is about 0.5 microns to about 25 microns. In some embodiments, the arc-shaped aspiration vias comprise an arc gap. In some embodiments, the arc gap may comprise an arc gap length that is 20% of the well boundary shape diameter. In some embodiments, the arc gap length may be 20 microns and the well boundary shape diameter may be 100 microns.

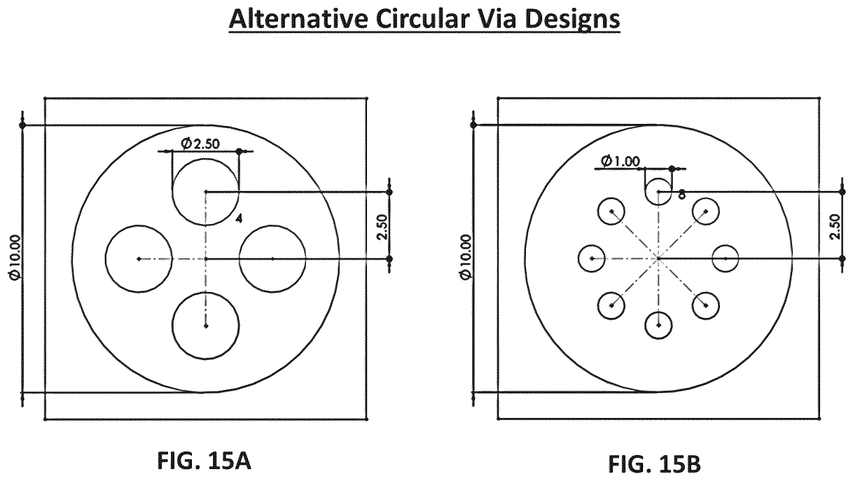

In some embodiments, the port shape or aspiration via shape may comprise a circle as shown in FIGS. 15A and 15B. In some embodiments, the aspiration via may comprise a plurality of aspiration vias. In some embodiments, the well of the capture site may comprise from 1 aspiration via to 20 aspiration vias. In some embodiments, the well of the capture site may comprise four aspiration vias. In some embodiments the well of the capture site may comprise 4 circular shaped aspiration vias as depicted in FIG. 15A. In some embodiments the well of the capture site may comprise 8 circular shaped aspiration vias as depicted in FIG. 15B. In some embodiments, the circle-shaped aspiration vias are arranged in an annular pattern as depicted in FIG. 15B. In some embodiments, the port or aspiration via comprises a diameter. In some embodiments, the port or aspiration via comprises a diameter about 1 micron to about 25 microns as depicted in FIG. 15B. In some embodiments the port or aspiration via comprises a diameter of about 1 micron as depicted in FIG. 15B. In some embodiments the port or aspiration via comprises a diameter of about 2.5 microns as depicted in FIG. 15A.

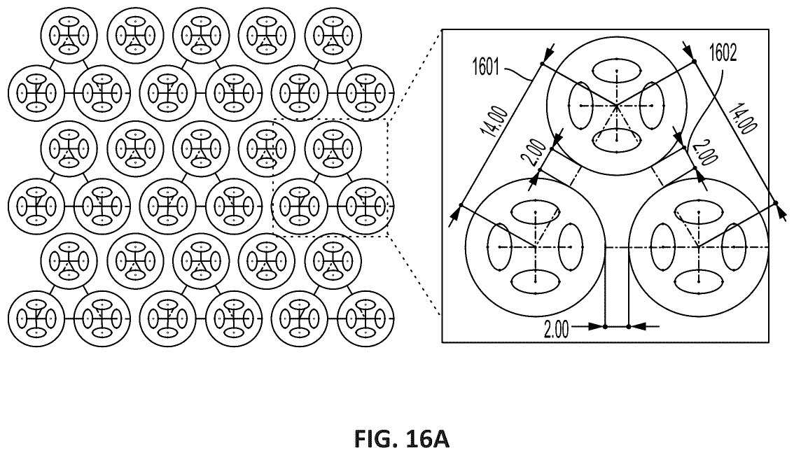

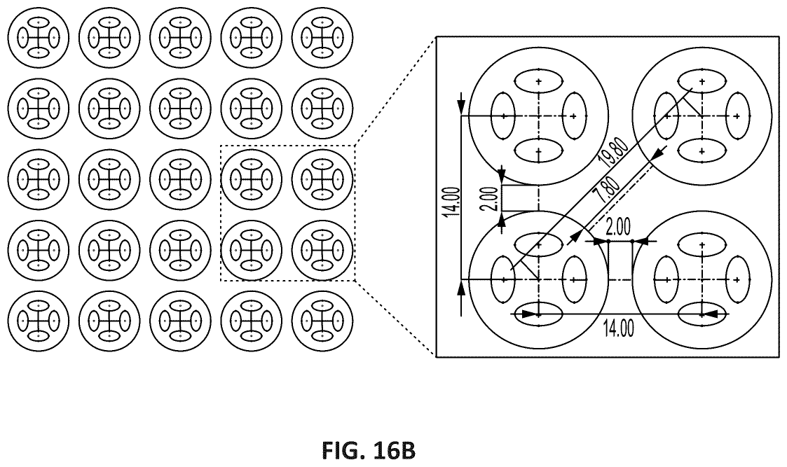

FIG. 16A-16B show non-limiting examples of capture site packing density and patterning. In some embodiments, the capture sites are packed in a linear packing pattern. In some embodiments, the linear packing pattern is a square-packed packing pattern as depicted in FIG. 16B. In some embodiments, the packing pattern is a face-centered pattern (e.g., diamond or hexagonal) as depicted in FIG. 16A. In some embodiments, the packing pattern is hexagonal as depicted in FIG. 16A

FIG. 16A-16B show non-limiting examples of capture site packing density, patterning and capture site (e.g., well) spacing. In some embodiments, a sub-array 204 comprises a well-to-well 206 spacing. In some embodiments, the well-to-well spacing refers to the shortest distance between a point on a first well boundary 207 (e.g., perimeter) and the closest point on the perimeter of a well in a second capture site, where the perimeter is the perimeter of the well at the surface 202 of the device 200. In some embodiments, the well-to-well spacing is about 0.1 micron to about 100 microns.

In some embodiments, the capture sites 205 are packed in a diamond packed-pattern (e.g., hexagonal). In some embodiments the adjacent well-to-well spacing is 2 microns for diamond packed-pattering, as depicted in FIG. 16A. In some embodiments, the center-to-center spacing is 14 microns as depicted in FIG. 16A.

In some embodiments, the capture sites 205 are packed in a square packed-pattern (e.g., square well packing). In some embodiments the adjacent well-to-well spacing 1602 is 2 microns and the diagonal well-to-well spacing is 7.80 microns in the linear (e.g., square-packed) pattern as depicted in FIG. 16B. In some embodiments, the adjacent center-to-center spacing 1601 is 14 microns and the diagonal center-to-center spacing is 19.80 microns as depicted in FIG. 16B.

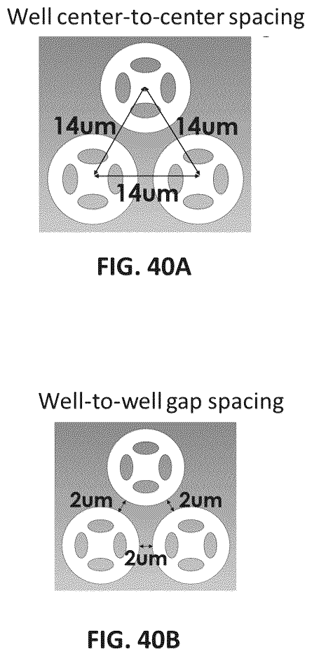

In some embodiments, the well center-to-center spacing refers to the distance between the center of a first well to the center of a second well, as depicted in FIG. 40A. In some embodiments, the well center-to-center spacing is not greater than 14 microns. In some embodiments, the well center-to-center spacing is 5 microns or less. In some embodiments, the well center-to-center spacing is 30 microns or greater. In some embodiments, the well center-to-center spacing is about 5 microns to about 30 microns. In some embodiments, the well center-to-center spacing is about 13.5 microns to about 14.5 microns. In some embodiments, the well center-to-center spacing is 14+/−0.5 microns. In some embodiments, the well center-to-center spacing is 14 microns as depicted in FIG. 40A.

In some embodiments, the well-to-well gap spacing (e.g., well-to-well spacing, well-to-well gap, or well boundary-to-well boundary gap) refers to the distance between the perimeter of a first well boundary to the perimeter of a neighboring well boundary, as shown in FIG. 40B. In some embodiments, the well-to-well gap spacing is 1 micron or less. In some embodiments, the well-to-well gap spacing is 10 microns or more. In some embodiments, the well-to-well gap spacing is about 1 to about 10 microns. In some embodiments, the well-to-well gap spacing is about 1.5 microns to about 2.5 microns. In some embodiments, the well-to-well gap spacing is about 2 microns. In some embodiments, the well-to-well gap spacing is 2+/−0.5 microns. In some embodiments, the well-to-well gap spacing is not greater than 2 microns.

In some embodiments, the well packing density is referred to as the number of wells, or equivalently, the number of capture sites, per unit area. In some embodiments, the well center-to-center distance, as measured horizontally and diagonally between neighboring wells, relates to the well packing density. In some embodiments, the sub-array of capture sites comprises a packing density. In some embodiments, the packing density may be dependent upon the well center-to-center spacing (e.g., capture site center-to-center spacing). In some embodiments, the wells (e.g., capture sites) are arranged in a hexagonal packing pattern (e.g., diamond packing pattern) as shown in FIG. 40A.

In some embodiments, as depicted in FIGS. 17A-17C a capture site 205 comprises an aspiration via channel depth (e.g., length) to well diameter ratio. In some embodiments, the capture site comprises an aspiration via depth (e.g., length) to aspiration minimum gap ratio.

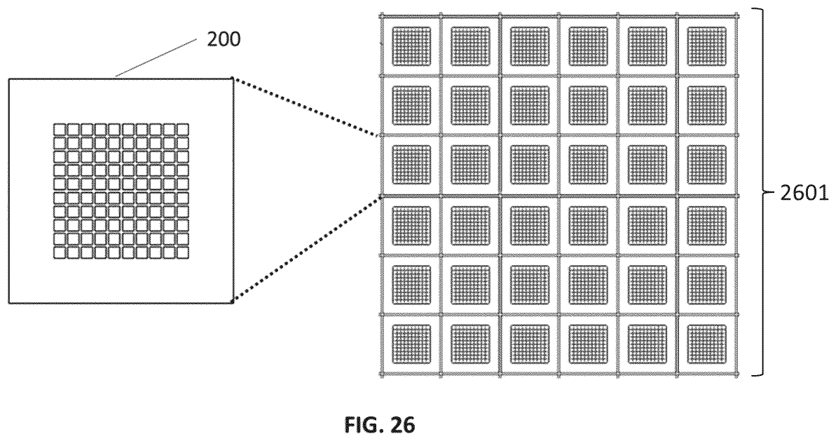

In some embodiments, the device 200 (e.g., chip) comprises an assembly of chips 2601 (e.g., an assembly of array of sub-arrays) as shown in FIG. 26.

### System

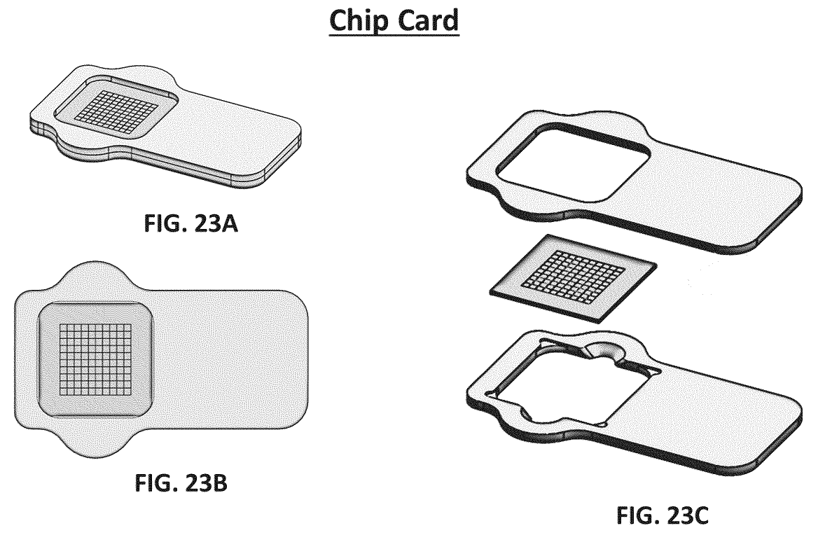

Described herein are various systems comprising the device described herein, reagents, a pump, or a combination thereof. In some embodiments, as depicted in FIG. 9A, the system comprises of a computer system 901, a positive pressure controller 903 and pump 902 to supply and control positive pressure. In some embodiments, as depicted in FIG. 9A the system comprises of a vacuum controller 903 and pump 902 to supply and control vacuum pressure. In some embodiments, the system comprises of reservoirs 905 for delivery of fluids into and out of the cartridge assembly 908 as depicted in FIGS. 9A-9B. In some embodiments, the system comprises of a link controller to display pressure and flow rate values. In some embodiments, the system comprises plastic tubing 904 to deliver fluids into the cartridge assembly 908. In some embodiments, the system comprises a 3-way valve 906 to switch between positive pressure into 913 the device and vacuum flow out 912 of the cartridge assembly 908. In some embodiments, the system comprises of a flow sensor 907 to observe flowrates. In some embodiments, the system comprises of a pressure sensor 911 to observe pressure. In some embodiments, the system comprises a cartridge assembly 908 that houses and provides sealing for the chip. In some embodiments, the system comprises of a chip card to hold the chip to enable protection and provide ease of use, as depicted in FIGS. 23A-23C. In some embodiments, the system comprises a chip that captures and perforates cells. FIG. 23A shows a 3D view of the chip card, according to some embodiments. FIG. 23B, shows a top-down view of the chip card according to some embodiments. FIG. 23C shows an exploded view of the chip card according to some embodiments.

### Methods

### Methods of Operation

Described herein are various embodiments of a method for the operation of devices and systems. Described herein are various embodiments of a method for the delivery of a payload to a cell. In some embodiments, the method comprises delivery of genetic material to a cell. In some embodiments, the method comprises delivery of genetic material to the interior of a cell. In some embodiments, the method comprises delivery of genetic material to the nucleus of a cell. In some embodiments, the method comprises delivery of genetic material to the interior of a nucleus of a cell. In some embodiments, the method comprises gene transfection. In some embodiments, the method provides for perforation of the cell membrane of a cell. In some embodiments, perforating the cell membrane creates a perforation in the cell membrane. In some embodiments, perforating the cell membrane creates a perforation (e.g., pore, poration, or hole) in the cell membrane, allowing for diffusion of the payload from the exterior of the cell, through the perforation and into the interior of the cell. In some embodiments, the method provides for perforating the cell membrane, creating a perforation (e.g., pore) in the cell membrane, the perforation allowing for diffusion of the payload from the exterior of the cell into the interior of the cell and/or the nucleus of the cell.

Described herein are various embodiments of a method comprising obtaining the system and/or the device as described herein; introducing the cell to the surface 202; applying a pressure-time waveform and allowing for capture of, perforation (e.g., poration) of and release of the cell and insertion a payload into the cell. In some embodiments, the pressure time waveform is configured for pulsatile flows (during capture/release flow) to improve cellular compression over the projection 209 and recoil away from the projection 209 for improvement and increase in perforation (e.g., poration) events.

In some embodiments, the cycle comprises a pulse. In some embodiments, the pressure-time waveform comprises the pulse. In some embodiments, the pressure time-waveform comprises a plurality of pulses. In some embodiments, the pressure time waveform comprising the plurality of pulses is referred to as a pulsatile waveform. In some embodiments, the pulsatile waveform is configured to produce pulsatile flow. In some embodiments, the pulsatile flow results in capture and release of the cell. In some embodiments, the pulsatile flow results in: capturing; compressing, perforating (e.g., porating), recoiling, inserting of payload and releasing of the cell, and wherein the capturing, compressing, perforating (e.g., porating), recoiling, inserting of payload and releasing of the cell may or may not occur at the same time, and may occur any order.

### Method of Fabrication

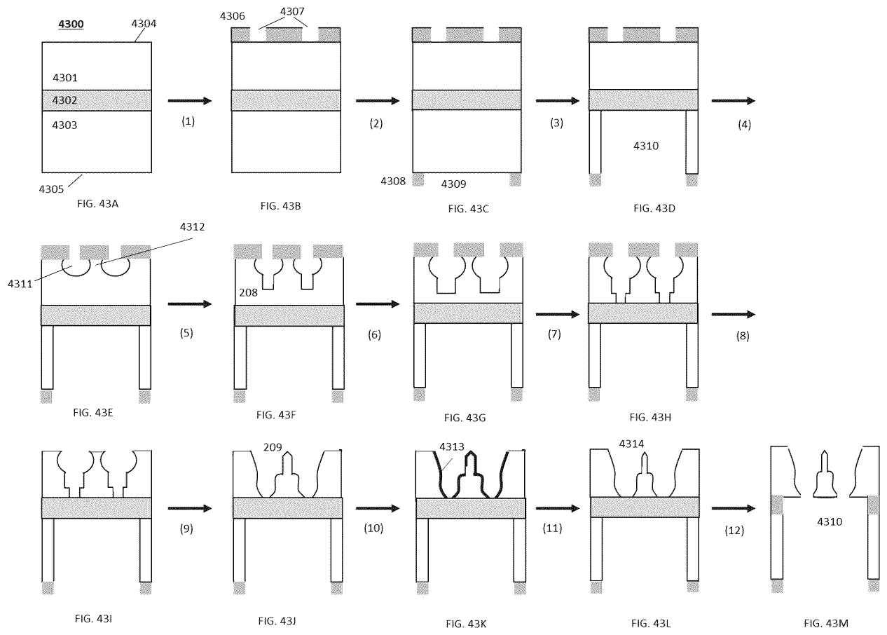

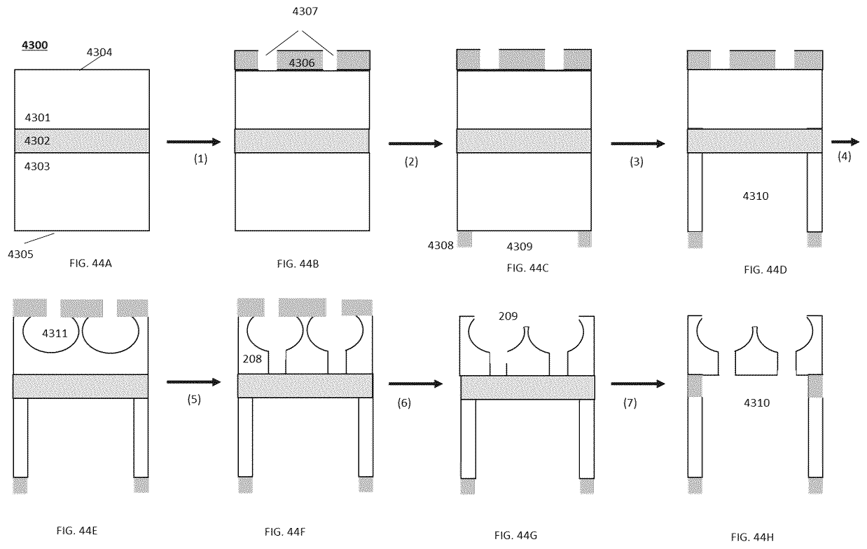

Described herein are various embodiments of methods for the fabrication of a device 200 (e.g., microneedle array) as depicted in FIG. 2. FIGS. 43 and 44 depict cross sectional views of various parts of the process of forming a device with a well, projection, and ports in accordance with some embodiments.

### Substrate

In some embodiments, a substrate may comprise a feature layer comprising one or more features, wherein the one or more features may comprise one or more wells, one or more projections, one or more ports (e.g., aspiration vias), or any combination thereof. In some embodiments, the substrate may comprise a structural layer. In some embodiments, the device may comprise one or more layers comprising single or multiple patterning layers, masking layers, feature layers, structural layers, or a combination thereof. In some embodiments, the layers may comprise silicon in single crystal (e.g., crystalline silicon), polycrystalline, or amorphous structure. In some embodiments, the layers may comprise crystalline silicon, polycrystalline silicon or amorphous silicon. In some embodiments the layers may comprise silicon nitride, silicon dioxide, buried oxide layer, silicon carbide, gallium arsenide, gallium phosphide, indium phosphide, indium arsenide, a semiconductor alloy, or any combination thereof. In some embodiments, a method for forming the oxide layer may comprise a deposition process. In some embodiments, the deposition process may comprise a plasma deposition process, a thermal oxidation deposition process, chemical vapor deposition (CVD) deposition process or any combination thereof.

In some embodiments, the patterning layer thickness may comprise a thickness. In some embodiments, the patterning layer may comprise a thickness of about 0.1 microns to about 10 microns. In some embodiments, the patterning layer may comprise a thickness of about 0.1 microns or greater. In some embodiments, the pattering layer may comprise a thickness of about 10 microns or greater.

In some embodiments, the masking layer may comprise a thickness. In some embodiments, the masking layer may comprise a thickness of about 0.1 microns to about 100 microns. In some embodiments, the masking layer may comprise a thickness of about 0.1 microns or less. In some embodiments, the masking layer may comprise a thickness of about 100 microns or more.

In some examples, the masking layer may comprise a thickness of at least about 0.1, 0.2, 0.3, 0.4, 0.5, 0.6, 0.7, 0.8, 0.9 1, 2, 3, 4, 5, 6, 7, 8, 9, 10, 20, 30, 40, 50, 60, 70, 80, 90 micron or more. In some examples, the masking layer may comprise a thickness of at most about 200, 100, 90, 80, 70, 60, 50, 40, 30, 20, 10, 9, 8, 7, 6, 5, 4, 3, 2, 1, 0.9, 0.8, 0.7, 0.6, 0.5, 0.4, 0.3, 0.2, or smaller.

In some embodiments, the feature layer may comprise a thickness. In some embodiments, the feature layer may comprise a thickness of about 0.1 microns to about 100 microns. In some embodiments, the feature layer may comprise a thickness of about 0.1 microns or less. In some embodiments, the feature layer may comprise a thickness of about 100 microns or more.

In some embodiments, the structural layer may comprise a thickness of about 0.1 microns to about 1000 microns. In some embodiments, the structural layer may comprise a thickness of 0.1 microns or less. In some embodiments, the structural layer may comprise a thickness of 1000 microns or more.

In some embodiments a substrate 4300 may comprise a device layer, an insulator layer, a handle layer, a masking layer, or any combination thereof. In some embodiments, the masking layer may comprise an oxide layer. In some embodiments, the masking layer may comprise silicon dioxide. In some embodiments the masking layer may comprise a 1-micron thick silicon dioxide layer.

In some embodiments, the substrate may comprise a handle layer and a device layer, wherein the device layer may comprise an insulator layer. In some embodiments, the device layer may comprise the insulator layer. In some embodiments, the device layer may comprise an oxide. In some embodiments, the device layer may comprise silicon dioxide.

### Patterning

In some embodiments of the method for fabrication, may comprise selectively exposing the substrate to various etchants through a mask. In some embodiments, the mask may comprise a hard mask. In some embodiments, the mask may comprise an oxide. In some embodiments, the mask may comprise a soft mask. In some embodiments, the soft mask may comprise a polymer. In some embodiments, a method for fabricating the mask may comprise photolithographically fabricating the soft mask. In some embodiments a hard mask is formed by a combination of photolithography followed by etching of the mask layer to expose regions of the substrate (e.g., surfaces of the device layer or handle layer) that were previously covered by the masking layer. In such embodiments, wherein a hard mask is formed, the photolithographically produced soft mask may be removed, revealing the hard mask.

In some embodiments, the soft mask (e.g., patterning layer) may comprise a thickness. In some embodiments, the soft mask may comprise a thickness of about 0.1 microns to about 10 microns. In some embodiments, the soft mask may comprise a thickness of about 0.1 microns or less. In some embodiments, the soft mask may comprise a thickness of about 10 microns or more.

In some embodiments, patterning may comprise alignment of a pattern on the back side of the wafer with a pattern on the front side of the wafer.

### Chemical Mechanical Polishing (CMP)

In some embodiments, methods may comprise chemical mechanical polishing (e.g., CMP). In some embodiments, methods may comprise CMP, wherein the CMP may comprise planarizing a substrate. In some embodiments, methods may comprise CMP, wherein the CMP may comprise thinning a substrate.

### Dry Etching/Milling

In some embodiments, the dry etching may be referred to as plasma etching. In some embodiments, dry etching may comprise anisotropic (e.g., in one direction) dry etching. In some embodiments, dry etching process may be configured to produce anisotropic trenches in silicon. In some embodiments, dry etching process may be configured to produce anisotropic trenches in an oxide. In some embodiments, dry etching process may be configured to produce anisotropic trenches in silicon oxide. In some embodiments, the dry etching may be referred to as milling. In some embodiments, the dry etching may comprise argon sputter etching, ion milling, plasma etching, or reactive ion etching. In some embodiments, the substrate may be oriented perpendicular to the direction of the etchant (e.g., argon ions). In some embodiments, the substrate may be oriented at an oblique angle relative to the etchant (e.g., argon ions). In some embodiments, the dry etching may comprise rotating the substrate. In some embodiments, the substrate may be oriented at an oblique angle relative to an etchant direction. In some embodiments, the dry etching may comprise orienting the substrate at an oblique angle relative to an etchant direction. In some embodiments, the dry etching may comprise orienting the substrate at an oblique angle relative to an etchant direction while rotating the substrate.

### Wet Etching

In some embodiments, wet etching may be used. In some embodiments, the wet etching may comprise isotropic (e.g., in all directions) wet etching. In some embodiments, the wet etching may be configured to isotropically etch silicon. In some embodiments, the isotropic wet etching of silicon may comprise an etchant comprising sodium hydroxide.

### Alpha Prototype Gen 1 Fab Process

Described herein are various embodiments of a method for fabricating a gene transfection device as depicted in FIGS. 43A-43M. In some embodiments, the method may comprise providing a substrate 4300 comprising a device layer (e.g., feature layer) 4301, an insulator layer (e.g., buried oxide layer or BOX layer) 4302, a handle layer (e.g., structural layer) 4303, or any combination thereof, as depicted in FIG. 43A.

In some embodiments, operation (1) may comprise forming a mask 4306 on the front surface 4304 of the substrate 4300 of FIG. 43A, operation (1) yielding the substrate 4300 of FIG. 43B. In such an embodiment, the mask 4306 may comprise openings 4307 exposing the device layer to etchants.

In some embodiments, operation (2) may comprise forming a mask 4308 on the back surface 4305 of the substrate 4300 of FIG. 43B, operation (2) yielding the substrate 4300 of FIG. 43C. In such an embodiment, the mask 4308 may comprise one or more openings 4309 exposing the handle layer (e.g., structural layer) 4303. In such embodiments, the one or more openings 4309 may be aligned to a sub-array 204 on the front side 4304 of the substrate. Such subarrays 204 can be seen in FIG. 2.

In some embodiments, operation (3) may comprise dry etching a back side sub-array trench 4310 into the handle layer 4303, operation (3) yielding the substrate 4300 as depicted in FIG. 43D.

In some embodiments, operation (4) may comprise isotropic wet etching of the front side 4304 of the substrate 4300. In such an embodiment, a well 4311 may be formed at each opening 4307 of the mask 4307 as depicted in FIG. 43E. In some embodiments, two or more wells 4311 are joined through isotropic etching to form a composite well. In some embodiments, the composite well may comprise a clover shaped well boundary perimeter due to the arrangement of the openings 4307. In some embodiments, the remaining substrate material at the center of the composite well may comprise a blunt projection 4312 as depicted in FIG. 43E.

In some embodiments, operation (5) may comprise anisotropically dry etching through the opening 4307 to form a straight walled channel in the center of each initial well 4311. In some embodiments, on or more straight walled channels may comprise one or more ports (e.g., aspiration vias) 208 as depicted in FIG. 43F.

In some embodiments, operation (6) may comprise isotropically dry etching a second time to widen the wells and channels, thereby narrowing the blunt projection 4312 as depicted in FIG. 43G.

In some embodiments, operation (7) may comprise anisotropically dry etching through the opening 4307 a second time to form a secondary inner port channel (e.g., aspiration via) 208 as depicted in FIG. 43H.

In some embodiments, operation (8) may comprise removing the mask 4306 by etching. In some embodiments, the mask 4306 may be an oxide based hard mask. In some embodiments, operation (8) yields the substrate 4300 as depicted in FIG. 43I. In some embodiments, the oxide based hard mask may be removed by dry etching. In some embodiments, the dry etching to remove the silicon dioxide hard mask may comprise selectively dry etching silicon dioxide over silicon. In some embodiments, the selectively dry etching silicon dioxide over silicon may refer to a process comprising etching silicon dioxide with an etchant comprising an etch rate for silicon dioxide that is greater than an etch rate for silicon. In some embodiments, the silicon dioxide etch rate is 2 times greater than the silicon etch rate. In some embodiments, the silicon dioxide etch rate is 2, 3, 4, 5, 6, 7, 8, 9, 10, or more times greater than the silicon etch rate.

In some embodiments, operation (9) may comprise ion milling (e.g., Argon sputter etching), operation (9) yielding the substrate as depicted in FIG. 43J. In some embodiments, the ion milling may comprise rotating the substrate. In some embodiments, the ion milling may comprise ion milling with an etchant. In some embodiments, the etchant may comprise Argon ions. In some embodiments, the ion milling may comprise orienting the etchant at an oblique angle relative to the surface of the substrate. In some embodiments, the oblique angle may be 45 degrees. In some embodiments, the ion milling of operation (9) may comprise orienting the etchant at an oblique angle relative to the surface of the substrate, while rotating the substrate. In some embodiments, operation (9) may be referred to as projection sharpening. In some embodiments, operation (9) may comprise rounding the edges of the well (e.g., composite well). In some embodiments, the blunt projection 4312 can be referred to as the sharpened projection, or projection 209 as depicted in FIG. 43J.

In some embodiments, operation (10) may comprise depositing an oxide layer 4313. In some embodiments the oxide layer may be titanium dioxide. In some embodiments, operation (10) may comprise the first part of a second projection sharpening protocol. The oxide layer can be seen in FIG. 43K.

In some embodiments, operation (11) may comprise removing the oxide layer 4313 by a wet etch. In some embodiments the wet etch of operation (11) may comprise a buffered oxide etch (e.g., BOE). In some embodiments, operation (11) may comprise the second part of a second projection/needle sharpening protocol, operation (11) yielding the substrate 4300 and sharpened projection 4314 as shown in FIG. 43L.

In some embodiments, operation (12) may comprise dry etching the insulator layer (BOX layer) 4302 to connect the aspiration via channels 208 on the front side of the substrate to the back side sub-array trench 4310 as depicted in FIG. 43M.

### Method of Fabrication—Next Gen 2 Fabrication Process

Described herein are various embodiments of a method for fabricating a gene transfection device as depicted in FIGS. 44A-44H. In some embodiments, the method may comprise providing a substrate 4300 comprising a device layer (e.g., feature layer) 4301, an insulator layer (e.g., buried oxide layer or BOX layer) 4302, a handle layer (e.g., structural layer) 4303, a front side 4304, a back side 4305, or any combination thereof, as depicted in FIG. 44A.

In some embodiments, operation (1) may comprise forming a mask 4306 on the front surface 4304 of the substrate 4300 of FIG. 44A, operation (1) yielding the substrate 4300 of FIG. 44B. In such an embodiment, the mask 4306 may comprise one or more openings 4307 exposing the device layer to etchants.

In some embodiments, operation (2) may comprise forming a mask 4308 on the back surface 4305 of the substrate 4300 of FIG. 44B, operation (2) yielding the substrate 4300 of FIG. 44C. In such embodiments, the mask 4308 may comprise one or more openings 4309 exposing the handle layer (e.g., structural layer) 4303. In such embodiments, the one or more openings 4309 may be aligned to a sub-array 204 on the front side 4304 of the substrate. Such subarrays 204 can be seen in FIG. 2.

In some embodiments, operation (3) may comprise dry etching a back side sub-array trench 4310 into the handle layer 4303, operation (3) yielding the substrate 4300 as depicted in FIG. 44D.

In some embodiments, operation (4) may comprise isotropic wet etching of the front side 4304 of the substrate 4300. In such an embodiment, a well 4311 may be formed at each opening 4307 of the mask 4307 as depicted in FIG. 44E. In some embodiments, multiple wells 4311 are joined through isotropic etching to form a composite well. In some embodiments, the composite well may comprise a clover shaped well boundary perimeter due to the arrangement of the openings 4307. In some embodiments, the remaining substrate material at the center of the composite well may comprise a blunt projection 4312 as depicted in FIG. 44E.

In some embodiments, operation (5) may comprise anisotropically dry etching through the opening 4307 to form a straight walled channel in the center of each initial well 4311. In some embodiments, one or more straight walled channels comprise one or more ports (e.g., aspiration vias) 208 as depicted in FIG. 44F.

In some embodiments, operation (6) may comprise removing the mask 4306 and exposing the projection 209 by dry etching yielding the substrate as shown in FIG. 44G. In some embodiments, the dry etching may comprise projection sharpening in accordance with some embodiments.

In some embodiments, operation (7) may comprise dry etching the insulator layer (BOX layer) 4302 to connect the aspiration via channels 208 on the front side of the substrate to the back side sub-array trench 4310 as depicted in FIG. 44H.

### Method of Manufacture for Surface Modification

Described herein are various methods comprising molecular and/or cell manipulation capabilities allowing for delivery, concentration, and/or mixing of cells and/or payloads (e.g., cargo) on or near the sub-array 204 of capture sites 205 before, during, and/or after payload insertion (e.g., transfection).

In some embodiments, the payload may be coated or adsorbed onto the device. In some embodiments, the device surface may be modified or coated with agents allowing for secondary payload coating or adsorption. In some embodiments, the coating method may comprise bulk adsorption, sputtering, evaporation, spotting, printing or electrodeposition methods.

### Numbered Embodiments

1. A system for precision single-cell mechanoporation comprising:
   a) a static array of wells, wherein each well of the array of wells is configured to capture a single cell of a single cell suspension therein;
   b) a static needle in the bottom surface of each well of the static array of wells configured to deliver or inject a cargo into the nucleus of the single cell captured in the well; and
   c) one or more modules for generating a pressure gradient, wherein the pressure gradient is configured to induce a pressure-driven flow of the single cell suspension in the system, thereby leading the single cell to get captured in the well;
   wherein capturing the single cell in the well is accomplished without centrifugation of the system or the static array.
2. The system of the previous embodiment, wherein the single cell is captured in the well via sedimentation and flow focusing of the single cell in the well.
3. The system of any one of the previous embodiments, wherein the static needle is configured to deliver or inject the cargo into a section or an organelle of the single cell.
4. The system of any one of the previous embodiments, wherein the static needle is configured to deliver or inject the cargo into the nucleus of the single cell.
5. The system of any one of the previous embodiments, wherein the static needle is configured to deliver or inject the cargo into the single cell without back-and-forth movement.
6. The system of any one of the previous embodiments, wherein the length of the needle to the depth of the well comprises a ratio of about 30% to about 60%.
7. The system of any one of the previous embodiments, wherein the length of the needle to the depth of the well comprises a ratio of about 45% to about 65%.
8. The system of any one of the previous embodiments, wherein the length of the needle to the depth of the well comprises a ratio of about 50%.
9. The system of any one of the previous embodiments, wherein the well has a diameter of at most about 20 micrometers (microns).
10. The system of any one of the previous embodiments, wherein the well has a diameter of at most about 15 micrometers (microns).
11. The system of any one of the previous embodiments, wherein the well has a diameter of at most about 9 micrometers (microns).
12. The system of any one of the previous embodiments, wherein the well has a diameter of at most about 8 micrometers (microns).
13. The system of any one of the previous embodiments, wherein the static array of wells comprises at least 15,000 wells.
14. The system of any one of the previous embodiments, wherein the static array of wells comprises at least 20,000 wells.
15. The system of any one of the previous embodiments, wherein the static array of wells comprises at least 100,000 wells.
16. The system of any one of the previous embodiments, wherein the static array of wells comprises at least 200,000 wells.
17. The system of any one of the previous embodiments, wherein the system is capable of processing at least 1000 cells per cycle.
18. The system of any one of the previous embodiments, wherein the system is capable of processing at least 2000 cells per cycle.
19. The system of any one of the previous embodiments, wherein the system is capable of processing at least 100,000 cells per cycle.
20. The system of any one of the previous embodiments, wherein the system is capable of processing at least 200,000 cells per cycle.
21. The system of any one of the previous embodiments, wherein the system is capable of processing at least about 15,000 cells per cycle.
22. The system of any one of the previous embodiments, wherein the system is capable of processing at least about 20,000 well per cycle.
23. The system of any one of the previous embodiments, wherein the system is capable of processing at least about 100,000 cells per cycle.
24. The system of any one of the previous embodiments, wherein the system is capable of processing at least about 200,000 cells per cycle.
25. The system of any one of the previous embodiments, further comprising a computer software for controlling the performance of the system.
26. The system of any one of the previous embodiments, wherein the one or more modules for generating a pressure gradient comprise a reservoir containing the single cell suspension, a pressure controller, a pump, and a pump power supply.
27. The system of any one of the previous embodiments, further comprising a valve, a flow sensor, and a computer with control software.
28. The system of any one of the previous embodiments, wherein the pump is connected to the pressure controller, and wherein the pressure controller is pneumatically connected to the reservoir.
29. The system of any one of the previous embodiments, wherein the well comprises a depth of at most about 20 micrometers (microns).
30. The system of any one of the previous embodiments, wherein the well comprises a depth of at most about 10 micrometers (microns).
31. The system of any one of the previous embodiments, wherein the well comprises a depth of at most about 9 micrometers (microns).
32. The system of any one of the previous embodiments, wherein the well comprises a depth of at most about 8 micrometers (microns).
33. The system of any one of the previous embodiments, wherein the static needle comprise a length of from 1 micron to 8 microns, from 2 microns to 4 microns, and/or from 3 microns to 4 microns.
34. A method of cell mechanoporation:
   a) providing or obtaining a mechanoporation system, wherein the mechanoporation system comprises:
      i. a static array of wells, wherein each well of the static array of wells is configured to capture a single cell therein;
      ii. a static needle in the bottom surface of each well of the static array of wells;
      iii. one or more modules for generating a pressure gradient in the system; and,
      iv. one or more liquid reservoirs;
   b) providing or obtaining a cell suspension in the one or more liquid reservoirs of (iv);
   c) generating a pressure gradient across the one or more liquid reservoirs of (iv) and the static array of wells (i) using the one or more modules of (iii), thereby generating a pressure-driven flow of the cell suspension from the reservoirs across the static array of wells, thereby capturing single cells from the cell suspension in the wells of the static array of wells, without centrifugation and generating a single captured cell in the well; and,
   d) creating a pore in the single captured cell and delivering a cargo into the single captured cell using the static needle of (ii).
35. The method of the previous embodiment, wherein capturing the single cell in the well comprises sedimentation and flow focusing of the single cell in the well.
36. The method of any one of the previous embodiments, wherein the cargo is delivered or injected into a section or organelle of the single cell.
37. The method of any one of the previous embodiments, wherein the cargo is delivered or injected into the nucleus of the single cell.
38. The method of any one of the previous embodiments, wherein the single captured cell remains viable after performing the method thereon.
39. The method of any one of the previous embodiments, wherein at least about 70% of a population of cells processed using the method remain viable.
40. The method of any one of the previous embodiments, wherein at least about 90% of a population of cells processed using the method remain viable.
41. The method of any one of the previous embodiments, wherein at least about 10% of the wells of the static array of wells capture a single cell therein upon performing the method.
42. The method of any one of the previous embodiments, wherein at least about 15% of the wells of the static array of wells capture a single cell therein upon performing the method.
43. The method of any one of the previous embodiments, wherein at least about 20% of the wells of the static array of wells capture a single cell therein upon performing the method.
44. The method of any one of the previous embodiments, wherein at least about 40% of the wells of the static array of wells capture a single cell therein upon performing the method.
45. The method of any one of the previous embodiments, wherein the cargo load delivered into individual cells among a population of cells processed using the method, is substantially uniform.
46. The method of any one of the previous embodiments, wherein a coefficient of variability (% CV) of cargo load across individual cells among a population of cells processed using the method is at most about 50%.
47. The method of any one of the previous embodiments, wherein a coefficient of variability (% CV) of cargo load across individual cells among a population of cells processed using the method is at most about 40%.
48. The method of any one of the previous embodiments, wherein a coefficient of variability (% CV) of cargo load across individual cells among a population of cells processed using the method is at most about 30%.
49. The method of any one of the previous embodiments, wherein a coefficient of variability (% CV) of cargo load across individual cells among a population of cells processed using the method is at most about 20%.
50. The method of any one of the previous embodiments, wherein a coefficient of variability (% CV) of cargo load across individual cells among a population of cells processed using the method is at most about 10%.
51. The method of any one of the previous embodiments, wherein a coefficient of variability (% CV) of cargo load across individual cells among a population of cells processed using the method is at most about 5%.
52. The method of any one of the previous embodiments, wherein the static needle comprise a length of from 1 micron to 8 microns, from 2 microns to 4 microns, and/or from 3 microns to 4 microns.
53. A device, comprising: a substrate comprising:
   a. a surface; and
   b. a capture site, the capture site comprising:
      i. a well having a well boundary shape at the surface and having a well depth, the well configured to support a cell; and
      ii. a projection extending from the bottom of the well toward the surface of the substrate, the projection comprising a projection length, and
         a. wherein the projection length and the well depth comprise a projection length-well depth ratio.
54. The device of any one of the preceding embodiments, wherein the projection length-well depth ratio is about 25% to about 100%,
55. The device of any one of the preceding embodiments, wherein the projection length-well depth ratio is about 40% to about 60%.
56. The device of any one of the preceding embodiments, wherein the projection length-well depth ratio is about 50%.
57. The device of any one of the preceding embodiments, wherein the well boundary shape comprises a circle, ellipse, square, rectangle, triangle, or any combination thereof.
58. The device of any one of the preceding embodiments, wherein the well boundary shape comprises a boundary shape area.
59. The device of any one of the preceding embodiments, wherein the well boundary shape comprises a well diameter.
60. The device of any one of the preceding embodiments, wherein the well diameter is about 10 microns to about 15 microns.
61. The device of any one of the preceding embodiments, wherein the well diameter is about 10 microns.
62. The device of any one of the preceding embodiments, wherein the port comprises a port boundary shape.
63. The device of any one of the preceding embodiments, wherein the port boundary shape comprises a circle, ellipse, square, rectangle, triangle, arc, or combination thereof.
64. The device of any one of the preceding embodiments, wherein the port boundary shape comprises a long axis.
65. The device of any one of the preceding embodiments, wherein the port comprises a plurality of ports.
66. The device of any one of the preceding embodiments, wherein the port comprises an aspiration via.
67. The device of any one of the preceding embodiments, wherein the port comprises a lumen.
68. The device of any one of the preceding embodiments, wherein the projection comprises the lumen.
69. The device of any one of the preceding embodiments, wherein the plurality of ports comprises a lumen and an aspiration via.
70. The device of any one of the preceding embodiments, wherein the port area and boundary shape area comprise a port area-boundary shape area ratio.
71. The device of any one of the preceding embodiments, wherein the projection length is about 3 to about 4 microns.
72. The device of any one of the preceding embodiments, wherein the projection length is about 3.5 microns.
73. The device of any one of the preceding embodiments, wherein the well depth is from about 4 microns to about 6 microns.
74. The device of any one of the preceding embodiments, wherein the well depth is about 5 microns.
75. The device of any one of the preceding embodiments, wherein the handle layer thickness is from about 600 microns to about 700 microns.
76. The device of any one of the preceding embodiments, wherein the handle layer thickness is at least about 650 microns.
77. The device of any one of the preceding embodiments, wherein the handle layer thickness is about 675 microns.
78. The device of any one of the preceding embodiments, wherein the device layer thickness is about 25 microns to about 35 microns.
79. The device of any one of the preceding embodiments, wherein the device layer thickness is at least about 25 microns.
80. The device of any one of the preceding embodiments, wherein the device layer thickness is about 30 microns.
81. A system for gene transfection, the system comprising:
   a. a device as described in any one of the previous embodiments; and
   b. a pressure source;
   wherein, the pressure source is configured to produce a pressure time-waveform allowing for the capture of, perforation of, payload insertion to and release of a cell.
82. A method for gene transfection, comprising:
   a. obtaining the system or the device as described in any one of the preceding embodiments;
   b. introducing the cell to the surface;
   c. applying a pressure-time waveform and allowing for capture of, perforation of and release of the cell; and allowing for insertion of a payload into the cell.
83. A method for gene transfection, comprising:
   a. obtaining a system or device as described in any one of the preceding embodiments;
   b. introducing a cell to the surface;
   c. applying a pressure-time waveform and allowing for capture, perforation, insertion of a payload into the cell and release of the cell.
84. The method of any one of the preceding embodiments, wherein the pressure-time waveforms are applied across the port area.
85. The method of any one of the preceding embodiments, wherein the port comprises at least one port.
86. The method of any one of the preceding embodiments, wherein the pressure-time waveform comprises, a square shaped, a pulse-shaped, a sinusoidal-shaped, a chirp-shaped or any shape of waveform.
87. The method of any one of the preceding embodiments, wherein the pressure-time waveform comprises a maximum pressure of about 300 to about 500 mbar
88. The method of any one of the preceding embodiments, wherein the pressure-time waveform comprises a duration of about 10 minutes to about 20 minutes.
89. The method of any one of the preceding embodiments, wherein the pressure-time waveform comprises at least one cycle.
90. The method of any one of the preceding embodiments, wherein the cycle comprises a pulse.
91. The method of any one of the preceding embodiments, wherein the pressure-time waveform comprises the pulse.
92. The method of any one of the preceding waveforms, wherein the pressure time-waveform comprises a plurality of pulses.
93. The method of any one of the preceding embodiments, wherein the pressure time waveform comprising the plurality of pulses is referred to as a pulsatile waveform.
94. The method of any one of the preceding embodiments, wherein the pulsatile waveform is configured to produce pulsatile flow.
95. The method of any one of the preceding embodiments, wherein the pulsatile flow results in capture and release of the cell.
96. The method of any one of the preceding embodiments, wherein the pulsatile flow results in:
   a. capturing;
   b. compressing;
   c. perforating;
   d. recoiling;
   e. inserting of payload; and
   f. releasing of the cell, and
      iii. wherein the capturing, compressing, perforating, recoiling, inserting of payload and releasing of the cell may or may not occur at the same time, and may occur in any order.
97. The device of any one of the preceding embodiments, wherein the well diameter is about 12 microns.
98. The device of any one of the preceding embodiments, wherein the well depth is about 6 microns.
99. The device of any one of the preceding embodiments, wherein the handle layer thickness is about 610 microns.

### EXAMPLES

The following illustrative examples are representative of embodiments of the devices, systems, and methods described herein and are not meant to be limiting in any way.

### Example 1—Gene Transfection Method (Method of Operation)

In this example a user obtains a device as shown in FIGS. 1A-1F and 2. The user also obtains cells and reagents, including genetic materials, to be inserted into the cells. The packaged device (e.g., device loaded in a flow cell or cartridge assembly 908) is fluidly connected to the system as shown in FIG. 9A. The reagents and cells are loaded into the system through inlet ports that are fluidly connected to the packaged device and allow for contacting the surface 202 of the device 200 comprising the array of sub-arrays 201, where the sub-arrays 204 comprise capture sites 205. The controller of the system is programmed for the proper parameters for the capture of, perforation of, release of and diffusive transport of the genetic material into the interior of the cell's nucleus as shown in FIG. 1A. The cartridge assembly 908 is primed with phosphate buffered saline (PBS) buffer. Cells are flown through the fluidic circuit and across the surface 202 of the device 200. The pump is fluidly connected to the packaged device, specifically to the device aspiration vias 208 as shown in FIG. 9A. Cells are introduced into the inlet 909 of the cartridge assembly. Capillary action allows the cells to flow across the array of sub-arrays 201 of the device 200 housed within the cartridge assembly 908. When the flowing cells are in proximity of the capture sites 205, the controller 903 actuates the pump 902 to apply pulsatile pressure waveform comprising negative (e.g., vacuum) pressure from 35 to 85 mbar over a period of 2 minutes. The ramp rate to maximum pressure of the pulsatile pressure waveform is 10 mbar per second. The pulsatile vacuum pressure waveform applied through the aspiration vias 208 causes a net flow toward the direction of the aspiration vias, allowing for capture of a single cell into each capture site 205 of the sub-array of capture sites 204. Upon contacting the capture site 205 the single cell is perforated by the projection 209 (e.g., needle). In this experiment, the device comprises a projection 209 with a projection length 210 of 3.5 microns and a well depth 212 of about 7.5 microns, as shown in FIG. 17B. After one minute, the controller actuates the pump to apply a positive pressure wave to the aspiration vias, allowing for release of the cells from the capture sites. The released, perforated (e.g., porated) cells are then exposed to a payload of nucleic acids in the surrounding solution. Diffusion of the payload, through the pores allows for insertion of the payload into the nucleus of the cells.

### Example 2: Alpha Prototype Capture Site Design

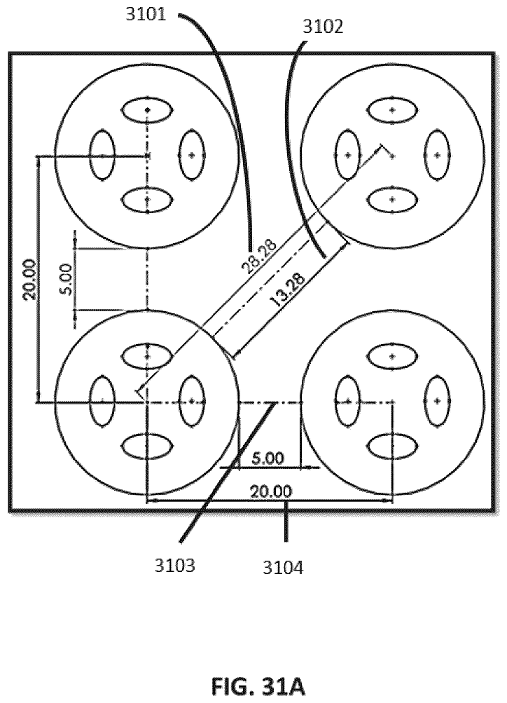

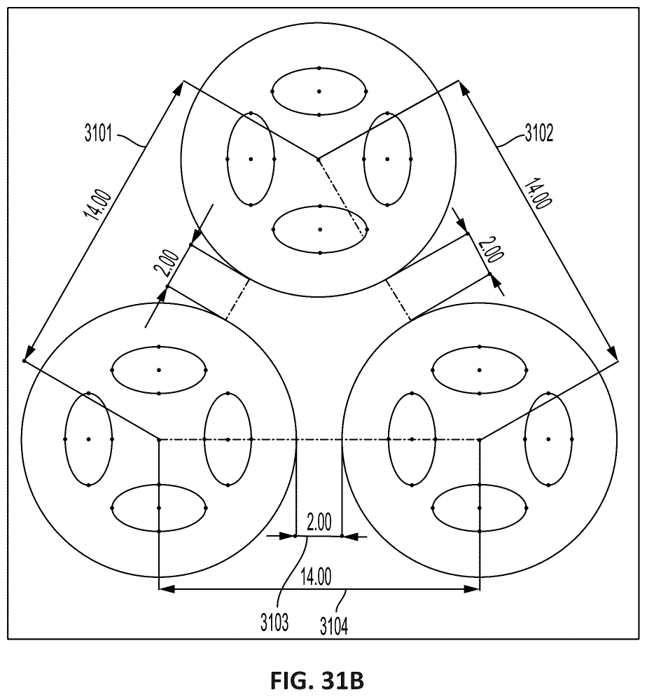

In this example, the capture site 205 comprises a circular well boundary shape 206 and a well boundary shape diameter of 15 microns FIG. 10A. This design capture site design 300 is referred to as the Alpha Prototype capture site design as shown in FIG. 3A. The well depth is 7.5 microns and the projection length 210 is 7.5 microns, yielding a projection length-well depth ratio of 100% FIG. 3A. The capture site comprises 4 elliptical aspiration ports FIG. 3A in a concentric port arrangement 600 (also shown in FIG. 6A). The aspiration port channel length is 22.5 microns FIG. 17A and the port boundary shape area is 25 microns squared FIG. 11A. The port boundary shape area is 25 microns squared, and the well boundary shape area is 176 microns squared, yielding a port boundary shape area to well boundary shape area of 1:7 FIG. 17A. The minimum gap between aspiration ports 301 is 2 microns. The elliptically shaped aspiration via exhibits a minimum diameter 302. The capture site sub-array exhibits square packing/patterning, adjacent well-to-well spacing of 5 microns, diagonal well-to-well spacing of 13 microns, adjacent center-to-center spacing of 20 microns, and diagonal center-to-center spacing of 28 microns. A cross section can be seen in FIG. 17A. In FIG. 31A diagonal center-to-center spacing 3101, diagonal well-to well spacing 3102, adjacent well-to-well spacing 3103 and adjacent center-to-center spacing 3104 are shown for a square packed sub-array of capture sites containing elliptically shaped aspiration vias.

### Example 3: Next Gen Prototype V2—12E Design

In this example, the capture site 205 comprises a circular well boundary shape 206, well boundary shape diameter of 12 microns FIG. 10B, a well depth of 6 microns, a projection length of 3.5 microns, and a projection length-well depth ratio of 1:2 FIG. 3B. This design capture site design 304 is referred to as the “Next Gen Prototype V2—12E” design. The capture site of the Next Gen Prototype V2—12E design comprises 4 elliptical aspiration ports of 4 microns (long axis) and 2 microns (short axis) FIG. 3B in a concentric port arrangement 600, an aspiration port channel length of 24 microns FIG. 17B, a port boundary shape area of 25 microns squared FIG. 11B, a port boundary shape area (25 microns squared), and a well boundary shape area (113 microns squared), yield a port boundary shape area to well boundary shape area ratio of 1:4.5. The minimum gap between aspiration ports 305 is 1 micron and the elliptically shaped aspiration via exhibits a minimum diameter 306 as shown in FIG. 3B. The capture site exhibits a hexagonal (e.g., diamond packing/patterning), an adjacent well-to-well spacing of 2 microns, a diagonal well-to-well spacing of 2 microns, an adjacent center-to-center spacing of 14 microns, and diagonal center-to-center spacing of 14 microns. The cross section of the capture site is depicted in FIG. 17B. In FIG. 31B diagonal center-to-center spacing 3101, diagonal well-to well spacing 3102, adjacent well-to-well spacing 3103 and adjacent center-to-center spacing 3104 are shown for a diamond packed (e.g., hexagonally packed) sub-array of capture sites containing elliptically shaped aspiration vias (e.g., ports).

### Example 4: Next Gen Prototype V2—Arc Via

In this example, the capture site 205 comprises a circular well boundary shape 206, a well boundary shape diameter of 10 microns FIG. 10C, a well depth of 5 microns, a projection length of 3.5 microns, and projection length-well depth ratio of 1:1.4 FIG. 3C. This capture site design 308 is referred to as the “Next Gen Prototype V2—Arc Via” design. The capture site comprises 3 arc shaped aspiration ports, a concentric port arrangement 600, an aspiration port channel length of 25 microns FIG. 17C, a port boundary shape area of 25 microns squared FIG. 11C, a port boundary shape area (25 microns squared) to well boundary shape area (78.5 microns squared) of 1:3, and a minimum gap between aspiration port 309 of 1 micron and the aspiration port thickness is 2 microns 310 as shown in FIG. 3C. The capture site exhibits a diamond packing/patterning, an adjacent well-to-well spacing of 2 microns, a diagonal well-to-well spacing of 2 microns, an adjacent center-to-center spacing of 12 microns, and a diagonal center-to-center spacing of 12 microns. The cross section of the capture is depicted in FIG. 17C. In FIG. 31C diagonal center-to-center spacing 3101, diagonal well-to well spacing 3102, adjacent well-to-well spacing 3103 and adjacent center-to-center spacing 3104 are shown for a diamond packed (e.g., hexagonally packed) sub-array of capture sites containing arc shaped aspiration vias.

### Example 5: Simulation of the Effect of Projection Length on Flow Velocity

A simulation to investigate the effects of projection length on flow velocity magnitude over the projection of the capture site, as shown in FIG. 21A, was carried out. In this experimental simulation flow velocity magnitude effects were simulated for the following projection lengths: 1.5, 2.5, 3.5, 4.5, 5.5, 6.5 and 7.5 microns as depicted in the series legend to the right of FIG. 21B. FIG. 21B plots the flow velocity magnitude over the projection in terms of height in microns, over the projection. As plotted in FIG. 21B, it is observed that the flow velocity magnitude over the projection is maximum for the shortest projection length of 1.5 microns. Additionally, the flow velocity magnitude decreases as the projection length increases.

### Example 6: Chip Card Assembly

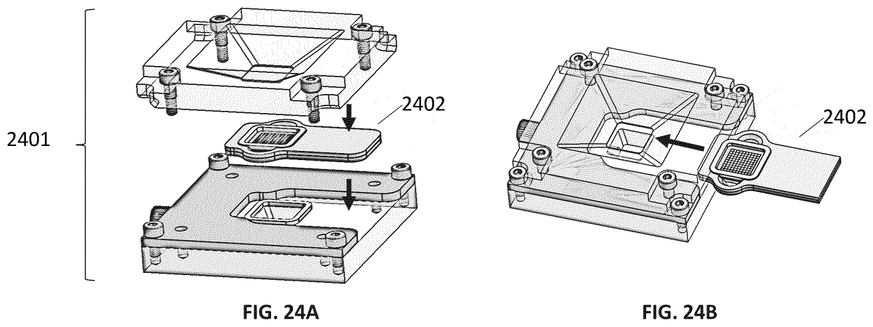

A chip card assembly 2401 is shown in two different forms as depicted in FIGS. 24A and 24B. A stacked assembly, wherein the chip card 2402 is bolted into the assembly, is shown in FIG. 24A. A slider assembly, wherein the chip card is slid into the chip card assembly is shown in FIG. 24B.

### Example 7: Cartridge Assembly

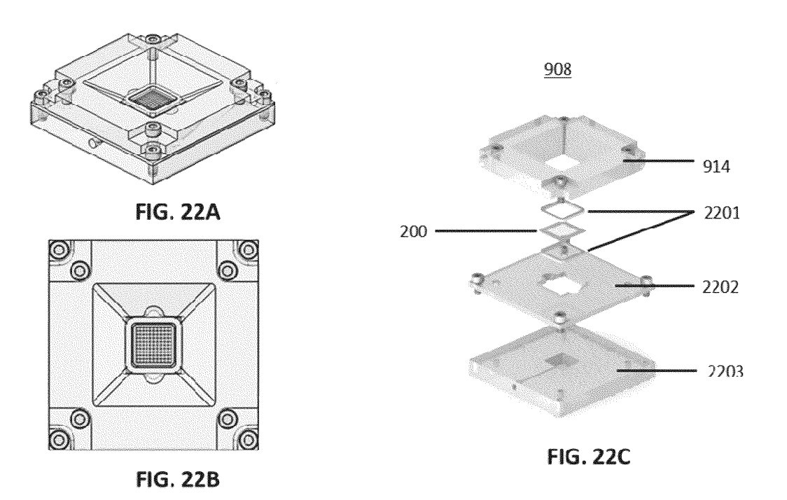

Examples of a cartridge assembly 908 connected to a constant pressure system schematic, under both negative pressure (e.g., capture flow direction) and positive pressure (e.g., release flow direction) are shown in FIGS. 9B-9C, respectively. As depicted in FIG. 9B, cells present in an exposed reservoir 914 are captured in the capture sites 205 by the flow driven by vacuum pressure 912 applied to the cartridge. As depicted in FIG. 9C, a positive pressure 913 is applied to allow for release of cells from the capture sites 205. FIG. 22A shows a 3D view of the cartridge assembly. FIG. 22B shows a top-down view of the cartridge assembly 908. FIG. 22C shows an exploded view of the cartridge assembly 908, comprising the device 200 a reservoir 914, gaskets 2201, a spacer 2202, and a manifold 2203.

### Example 8: System Unit Operations for the Celleto™ Device

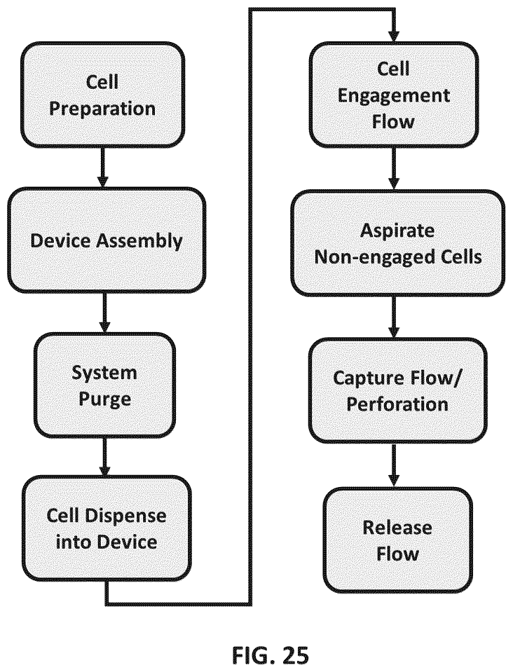

A system units operation protocol is shown in FIG. 25 for the Celleto™ device. In this example, the protocol comprises the following operations: cell preparation, device assembly, a system purge, cell dispensing into the device, cell engagement flow, aspirate non-engaged cells, capture flow/perforation and release flow.

### Example 9: Max Fluid Pressure Testing

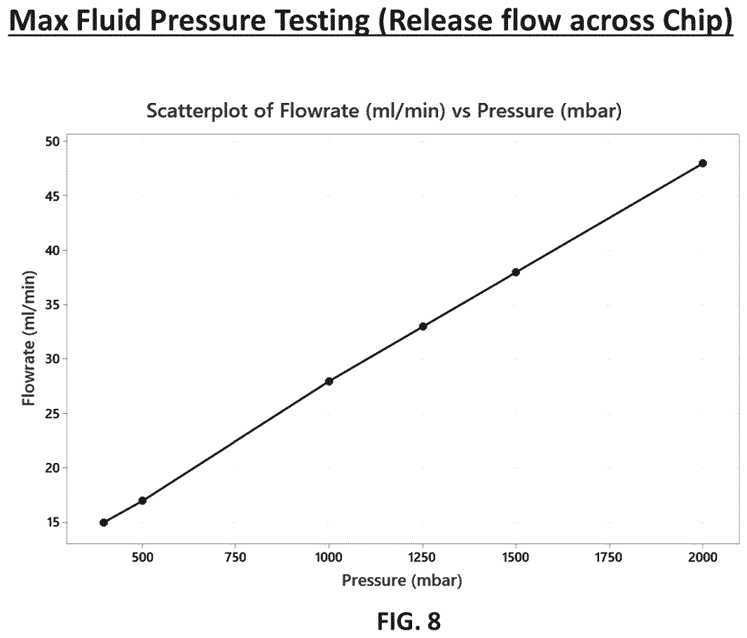

The Alpha Prototype device was tested against fluid pressure as applied by the system including the pressure controller 903 and the pump 902 as shown in FIG. 9A. In this example, a positive pressure was applied across the chip (e.g., device 200) to test release flow conditions (e.g., release of the cell from the device). FIG. 8 plots flow rate of fluid (out of the aspiration vias 208 and into the well of the capture site 205) along the y axis, versus the pressure applied in mbars, along the x-axis. The scatter plot represents the pressures applied with corresponding flow rates on the current Alpha Prototype chip simulating release flow. As the pressure was increased, the flowrate also increased linearly. The plot shows a max applied pressure of 2000 mbar and flowrates of up to 50 ml/min (which is the limit of the flow sensor). The chip was subjected to even higher pressures (up to 7000 mbar) and was able to withstand them with no damaging effects.

### Example 10: Maximum Principal Stress from Release Flow

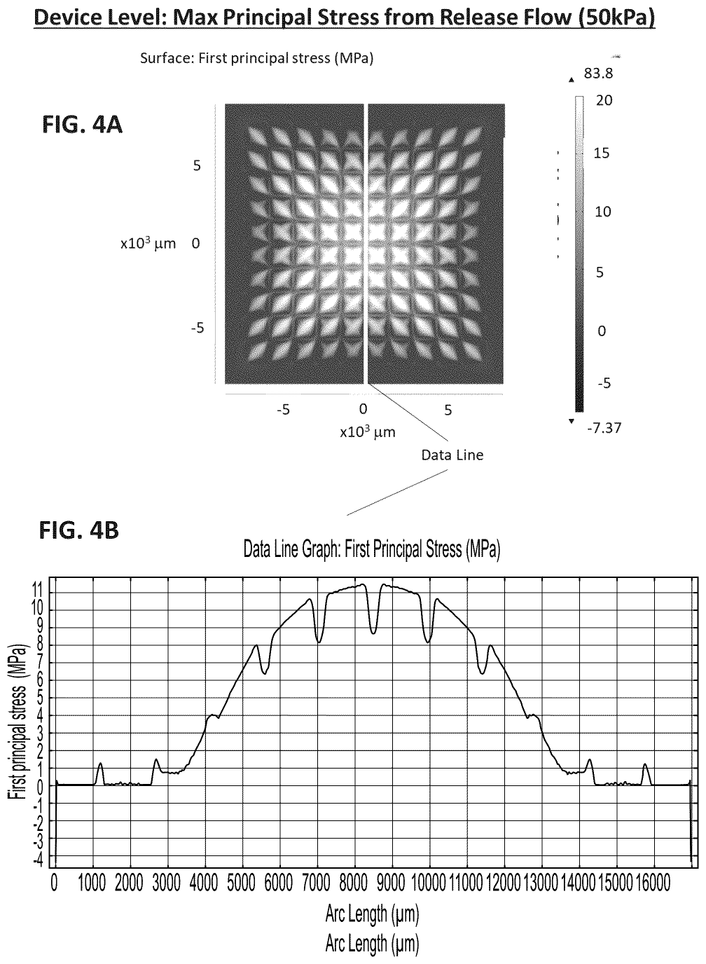

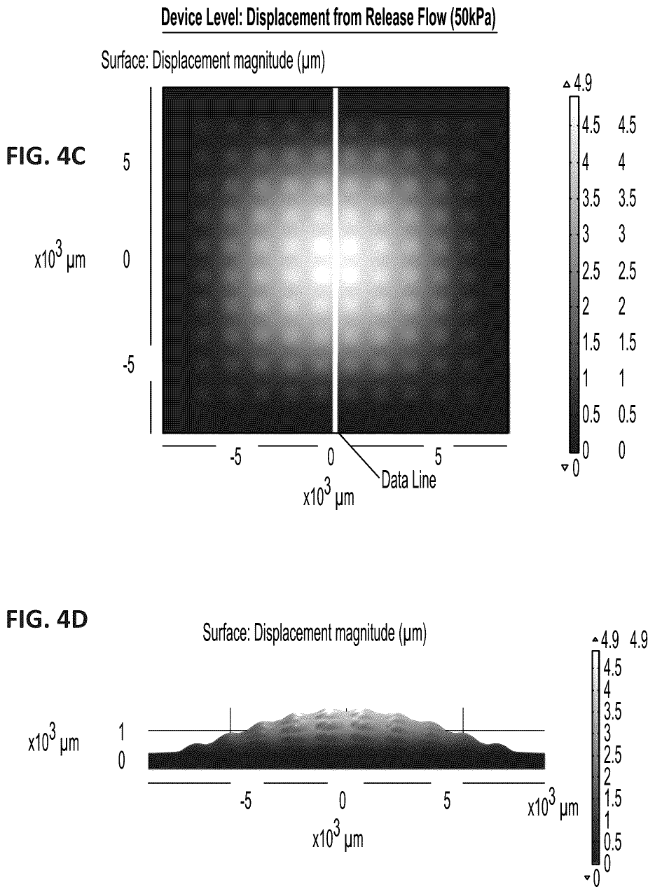

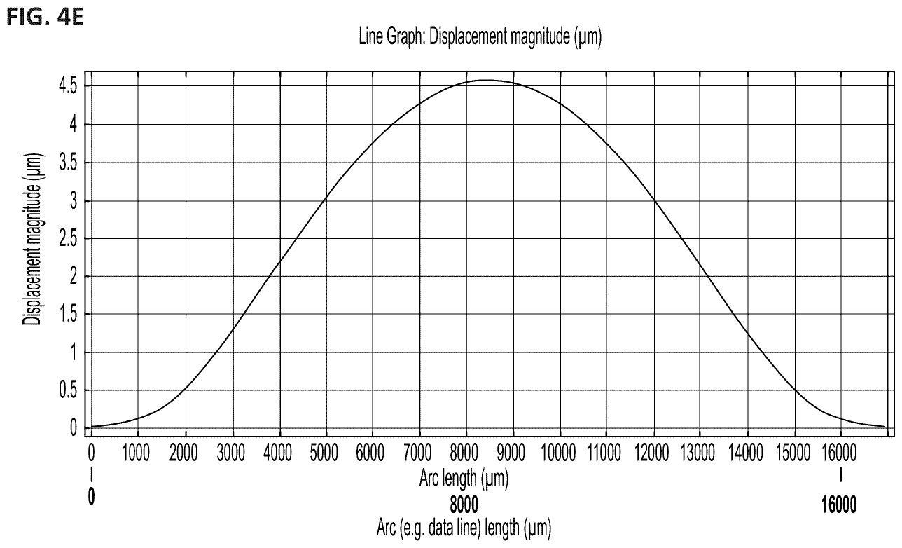

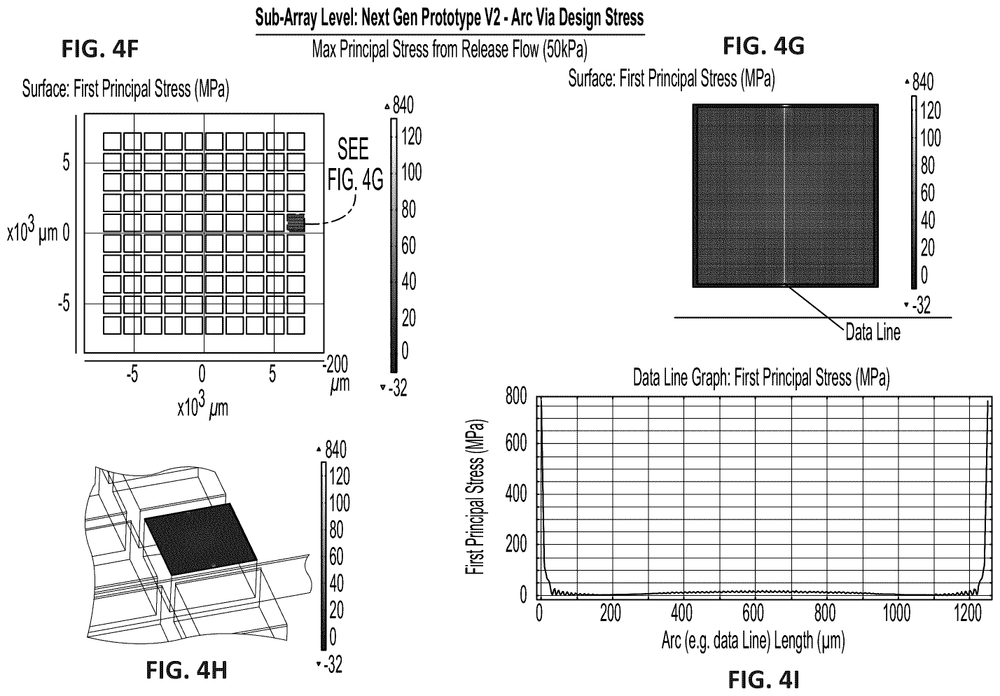

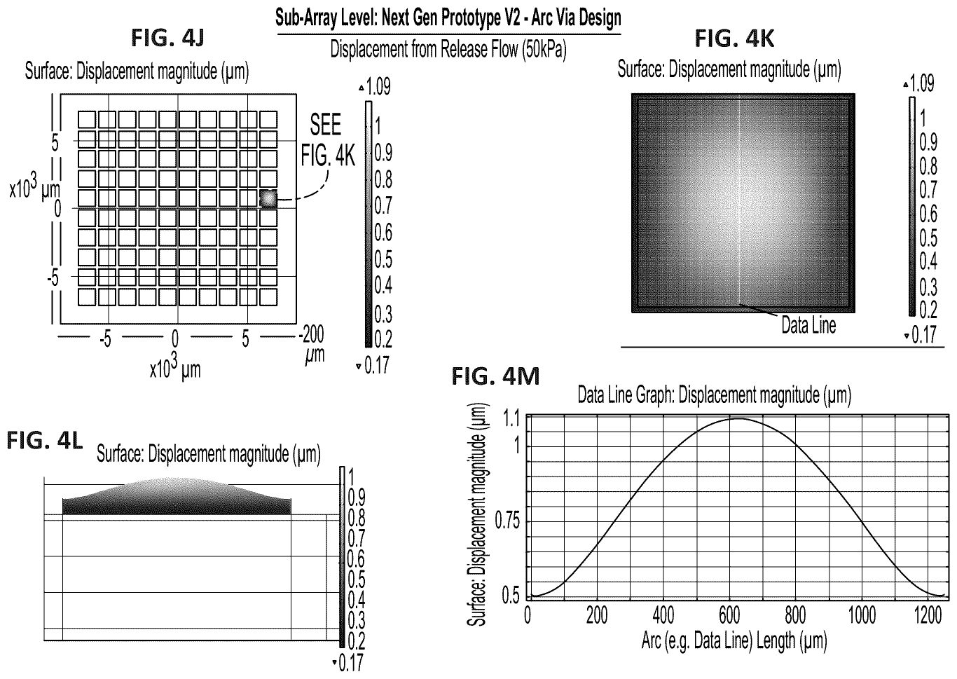

In this example, maximum principal stress and displacement magnitude values were calculated for a device exposed to 50 kPA of vacuum pressure to simulated release flow conditions as shown in FIGS. 4A-4M. FIGS. 4A-4B show a simulation for first principal stress across an entire device 200, in this case a device comprising one array of sub-arrays 201. FIG. 4A depicts a simulated surface of the array of sub-arrays, exhibiting first principal stress values (MPa) as shown in the heat map legend on the right. FIG. 4B depicts a line graph of first principal stress (MPa) versus arc length (e.g., distance across the array of sub-arrays) as called out in data line of FIG. 4A. From the plot in FIG. 4B, it was observed that the first principal stress was maximum at the center of the array of sub-arrays.

FIGS. 4C-4E show calculated displacement magnitude values across an array of sub-arrays 201, or chip, for the condition of 50 kPA of vacuum pressure applied to the device to simulate release flow. FIG. 4C depicts a top-down view of the array of sub-arrays, exhibiting displacement magnitude of the surface (m) as shown in the heat map legend on the right. FIG. 4D shows a cross section al view of the surface in FIG. 4C. FIG. 4E depicts a line graph of displacement magnitude (m) versus arc length (e.g., distance across the array of sub-arrays) as called out in the data line shown in FIG. 4C. From the plot in FIG. 4E, it was observed that the simulated displacement magnitude was maximum at the center of the array of sub-arrays.

FIGS. 4F-4I depict calculated maximum principal stress and displacement magnitude values results across a sub-array 204 of the array of sub-arrays 201. In this particular simulation, the device comprises the Next Gen Prototype V2—Arc Via capture site design. FIG. 4F shows a top-down view of a device 200 comprising an array of sub-arrays 201 and FIG. 4G shows a zoomed in sub-array 204. Calculated maximum, or first principal stress values are depicted in the legend to the right of FIG. 4G. FIG. 4H shows a zoomed in 3D oblique view of the device. FIG. 4I plots calculated first principal stress (MPa) along the data line called out in FIG. 4G.

FIGS. 4J-4M depict calculated displacement magnitude values results across a sub-array 204. In this simulation, the device comprises the Next Gen Prototype V2—Arc Via design. FIG. 4J shows a top-down view of a device 200 comprising an array of sub-arrays 201 and FIG. 4K shows a zoomed in sub-array 201. Calculated displacement magnitude (microns) of the surface when exposed to simulated vacuum pressure of 50 kPA is depicted in the legend to the right of FIG. 4K. FIG. 4L shows a cross section view of the sub-array 204. FIG. 4M plots calculated displacement magnitude (microns) along the data line as called out in FIG. 4K. From the plot in FIG. 4M, it was observed that the displacement magnitude was maximum at the center of the array of sub-arrays.

### Example 11: Simulation of Principle Stress at Center Edge of Device Sub-Array

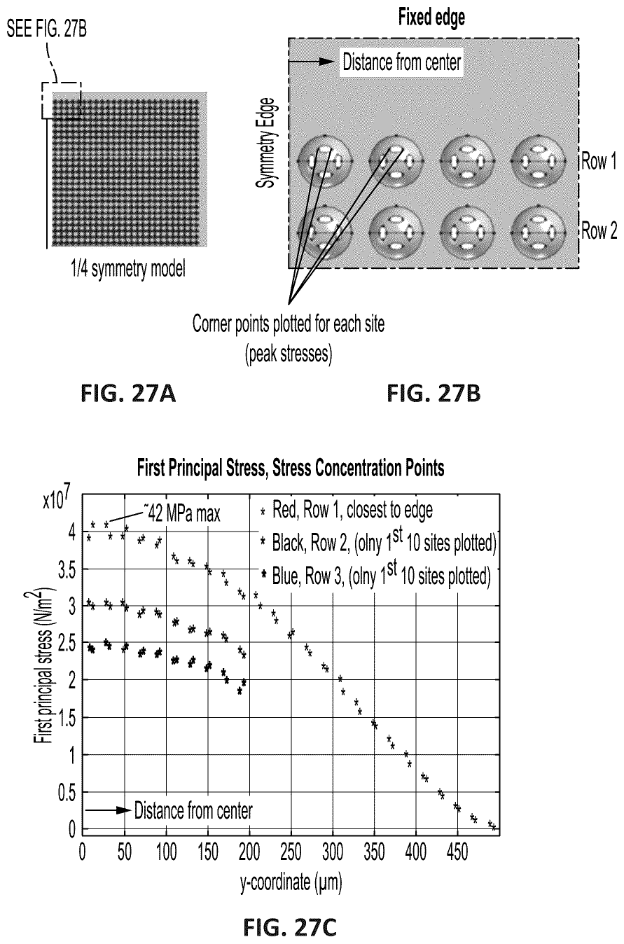

Application of pressure on the microfabricated structures (e.g., capture sites) of the device may cause mechanical stress on the substrate material, which in this example is silicon. The ability of the device to withstand pressures is especially critical given that large amounts of positive and negative pressures are exerted on the device layer of the substrate during operation of the device. Higher principal stresses due to structural elements fabricated into the substrate pose a higher chance of structural failure of the device. In this experiment, finite element analysis (FEA) design simulation using a 14 symmetry model was used to calculate maximum principal stresses at single sub-array level, as shown in FIGS. 27A-27C. FIG. 27A shows a schematic of a device sub-array 204. FIG. 27B shows a zoomed in region the device sub-array 204 outlined in a red box in FIG. 27A. Simulated peak stresses at aspiration via 208 apexes as indicated in in FIG. 27B are plotted in FIG. 27C. FIG. 27C plots first principal stress (N/m²) versus capture site 205 position in the sub-array 204 in terms of distance in microns from the center of the sub-array 204 edge. Three traces are shown in FIG. 27C: a red trace showing principal stress for aspiration via apexes in row 1 of FIG. 27B, closest to the edge; a black trace showing principal stress of each corner point of the first ten capture sites of row 2 of FIG. 27B; and a blue trace showing principal stress of each corner point of the first ten capture sites of row 3 (row 3 not shown FIG. 27B). From the plot of FIG. 27C, it is observed that principal stress at apexes of the elliptical vias of the capture sites drop as the distance from the center of the sub-array increases. Further, it was demonstrated that capture sites closest to the edge of the sub-array, in row 1, had the highest maximum stress at approximately 42 MPa as shown in FIG. 27C. Within row 1, the apexes were plotted as having the peak stresses. Rows 2 and 3 showed a significant amount of increasingly less stress, respectively. High stress values at these positions may explain why cleavage damage is observed along the edges of the sub-array.

### Example 12: Selective Removal of Capture Sites to Reduce Stress on the Device

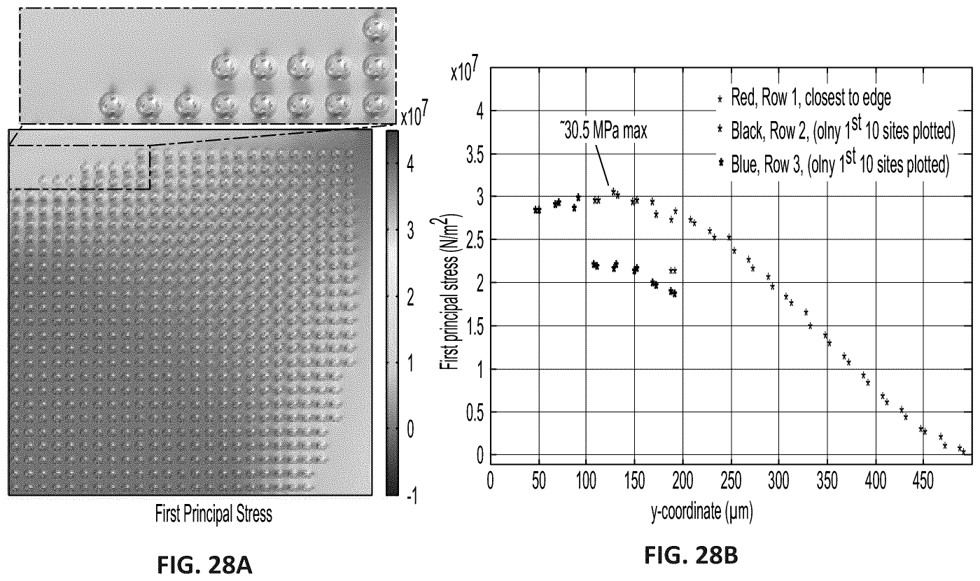

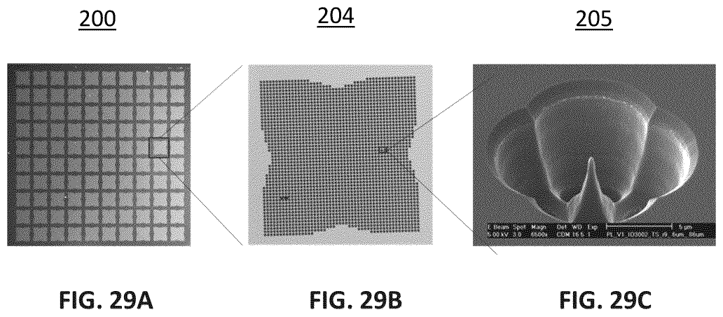

The purpose of the experiment described in this example was to demonstrate selective removal of capture sites from the sub-array to reduce principle mechanical stresses in the device. In this experiment, FEA design simulation was used to model the effects of the selective removal of capture sites, incorporating aspiration vias in the highest stress regions on principal stress of the device. FIG. 28A shows a sub-array 204 fabricated with selective removal of capture sites 205 in regions where the simulation in Example 10 showed high principal stress. Simulated principal stress for the capture sites outline in red in FIG. 28A is plotted in FIG. 28B. As in FIG. 27C, FIG. 28B plots first principal stress (N/m²) versus distance in microns of the capture site 205 from the center of the edge of the sub-array 204. Additionally, as in FIG. 27C, three traces are shown in FIG. 28B: a red trace showing principal stress for aspiration via apexes in row 1 of FIG. 28B, closest to the edge; a black trace showing principal stress of each corner point of the first ten capture sites of row 2 of FIG. 28B; and a blue trace showing principal stress of each corner point of the first ten capture sites of row 3. From the data plotted in FIG. 28B, it is seen that the simulated maximum principal stress was reduced from 42 MPa to 30.5 MPa, a 27% reduction. FIGS. 29A-29C show images of selective site removal approach implemented in fabricated device substrates. FIG. 29A shows a top-down bright-field microscope image of the full array of the device 200. FIG. 29B shows a zoomed in image of a single sub-array 204 of the device, with selective site removal of capture sites located at and around the center edges of the sub-array. FIG. 29C shows a scanning election microscope image of single capture site 205 fabricated in the silicon substrate 203 of the device 200. In this image, the projection 209 and at least one of the aspiration vias 208 is seen.

### Example 13—Optimization of Device and Handle Layer Thicknesses

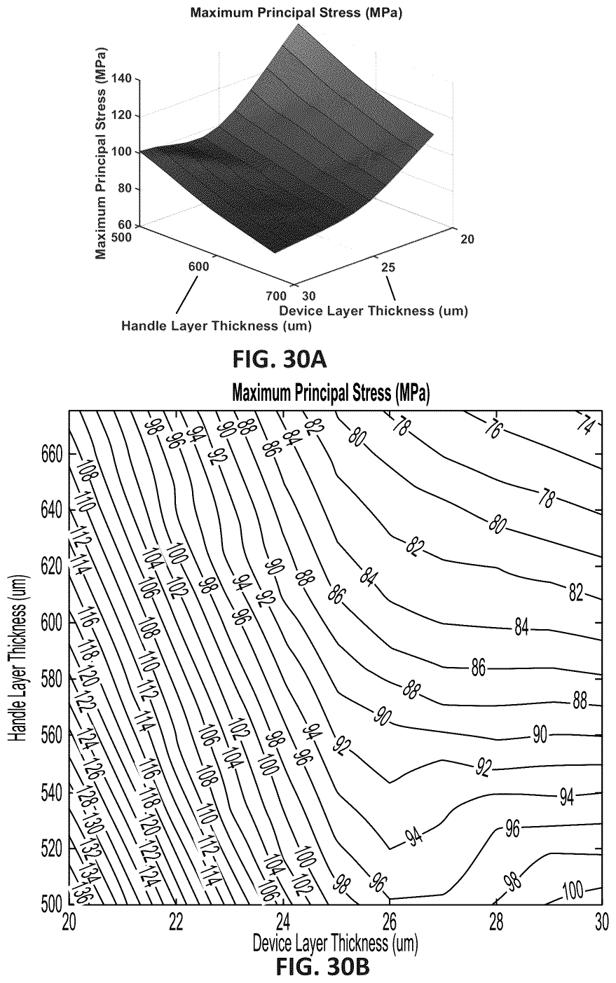

In this example, FEA design simulations (at full device level) are presented that show that increasing device and handle layer thicknesses reduce max principal stresses. FIG. 30A provides a three-dimensional plot of maximum principal stress (MPa) versus both the handle layer thickness (microns) and device layer thickness (microns). From the plot, one can observe that maximum principal stress decreases with both increasing handle layer thickness and with increasing device layer thickness. FIG. 30B provides a two-dimensional topological line plot of maximum principal stress (MPa) versus both handle layer thickness (microns) and device layer thickness (microns).

### Example 14—Constant Pressure System

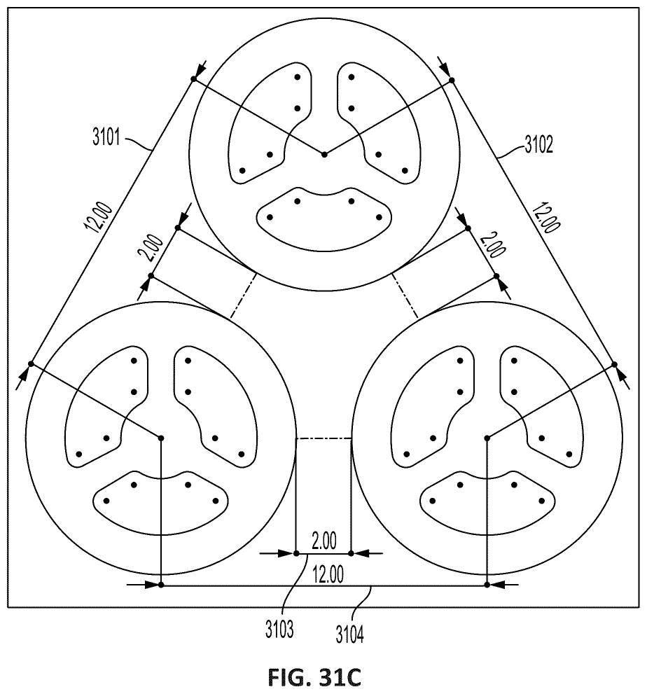

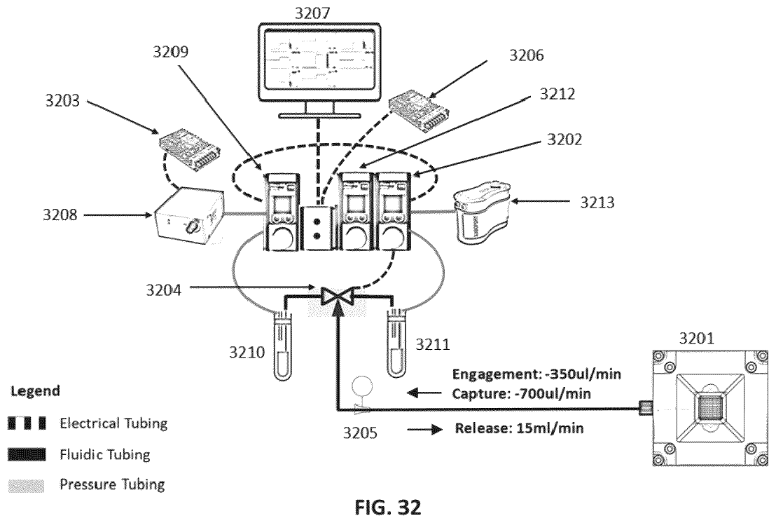

In this example a constant pressure system is described, as depicted in FIG. 32. The system includes a device 200 housed in a cartridge assembly 3201; buffer reservoirs; pressure controllers; a valve controller 3202, pumps; a pump power supply 3203; a 2-way valve 3204; a flow sensor 3205, a power supply (e.g., link module) 3206 and a computer with control software 3207. Linked to a pressure pump 3208 is a positive pressure controller 3209 that is fluidly connected to a first buffer reservoir 3210. The first buffer reservoir is fluidly connected to the cartridge 3201 containing the device. The cartridge is also fluidly connected to a second buffer reservoir 3211 that is fluidly connected to a negative pressure (e.g., vacuum) controller 3212 that is linked to the negative pressure (e.g., vacuum) pump 3213. To release cells from the device, the positive pressure controller actuates a pump 3208 to pressurize a gas acting on the buffer reservoir 3210 resulting in flow of buffer into the cartridge containing the device. In this case, pressurized gas acting on the buffer reservoir 3210 causes the buffer to flow into the cartridge (containing the device) at 15 milliliters per minute. The buffer flowing into the cartridge, flows through the aspiration vias 208 of each capture site 205 of the device 200 and pushes any captured cells out of the capture sites and into solution. To engage cells to the device, the negative pressure controller 3212 actuates a second pump (e.g., vacuum pump) 3213 to produce a negative pressure that acts on the second buffer reservoir 3211 resulting in a fluid flow out of the device. The buffer flowing out of, or into, the device flows through the aspiration vias of each well of each capture site, thereby pulling (e.g., engaging) the cells to the capture site. During engagement, the flow rate of the buffer is 350 microliters per minute out of, or into, the cartridge assembly. For capturing the cells to the device, the negative pressure controller 3212 produces additional negative pressure acting on the second buffer reservoir 3211 resulting in a flow rate (leaving the device) of 700 microliters per minute out of the device. Under the effect of this outward flow of buffer, individual cells are captured onto the projection 209 within each well of each capture site 205, allowing for poration of the cells by the projections. A 2-way flow valve 3204 is used to allow for buffer to flow into the cartridge 3201 at one time and to allow for buffer to flow out of the cartridge at another time. A flow sensor 3205 is included in the system to verify the flow rate of buffer flowing into or out of the cartridge.

### Example 15

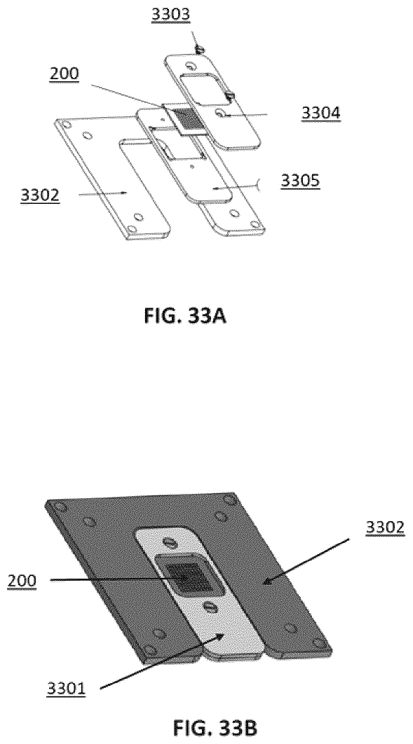

This example provides specifications and features for an exemplary device 200. FIG. 33A shows an exploded view diagram of the chip card that contains the device 200, a chip card with a top half 3304, a bottom half 3305, flat head screws 3303 to assemble the chip card top and bottom halves, and a spacer plate 3302. FIG. 33B shows an assembled view of an assembled chip card 3301, the device 200 contained within the chip card and the spacer plate that contains the chip card. The chip card houses the device 200 and minimizes direct user handling thus preventing damage. The thickness of the spacer plate controls the gap distance for a specific compression of the gasket. The compression of the gaskets from the reservoir and manifold plates provides for a robust, leak proof seal around the chip. In some examples, capture sites of the device were made to have a clover leaf well boundary shape. Both the apex of the lobe 3401 and the pinch point 3402 of the clover shape well boundary are labeled in FIG. 34A. Also, labeled in FIG. 34A are the well boundary shape perimeter (e.g., well perimeter) 207 and the ports (e.g., aspiration vias) 208. A tilt-view of the capture site is shown in FIG. 34B, where the capture site's relatively vertical side walls, saddles 3403, projection tip (e.g., needle tip) 3404, and projection base (e.g., needle base) 3405 are shown.

As shown in FIG. 35A, the well diameter was measured to be 12 microns. The verifiable acceptance criteria for the well diameter is 12+/−0.5 microns at the apex of the lobes of the clover leaf shaped well boundary perimeter (e.g., well perimeter) 3401. The well shape may also be a circle, but due to the etching process to make the aspiration vias, a clover leaf shaped well is formed. The well diameter is measured from the apex of one lobe of the clover leaf to the apex of the opposing lobe. For this example, the well is symmetric in both the horizontal and vertical directions as viewed in the image. As shown in FIG. 35B, the well depth of the capture site was measured to be 6 microns. The well depth was measured from the midpoint of the saddle 3403 to top surface of the device (e.g., plane of the well top surface). In FIG. 35B, the top surface of the device is identified by the axis line running between two pinch points 3402 of the clover leaf-shaped well boundary. The verifiable acceptance criteria for the well depth is 6 microns. The well depth is defined as the distance from the plane of the well top surface (e.g., device surface 202) down to the midpoint of a saddle. The plane of the top surface extends from the pinch point of the clover leaf to the pinch point of the opposing clover leaf. It is understood that there may be variances in the well depth due to the isotropic etch process. There is tolerance for variation in this specification as long as all other specs are met.

FIGS. 36A and 36B show top-down views and labeled description of the ports (e.g., aspiration via's) of a capture site. As shown in FIG. 36A the aspiration via gap 3605 was measured to be 1 micron. The verifiable acceptance criteria for the aspiration via gap is 1 micron +0.1, −0 microns. The port gap (e.g., aspiration via gap) is the closest distance between adjacent aspiration vias. This distance may not be less than 1 micron. The port via gap (e.g., aspiration gap) consistency was confirmed by inspecting FIB cross sections of capture sites throughout the device layer by SEM. As shown in FIG. 36B, the aspiration via size, as defined by the short axis (e.g., width) and long axis (e.g., length) of the elliptical aspiration via boundary, were measured to be 2 microns and 4 microns, respectively. The verifiable acceptance criteria for the elliptical shaped aspiration via long axis is 4 micron+0.1, −0.2 microns and for the short axis is 2 micron +0.1, −0.2 microns. There are 4 aspiration vias at the bottom of each well distributed concentrically around the well center point. It is presumed, due to the isotropic etch process that created the wells, that the bottom is relatively level. An aspiration via (e.g., port) is an elliptically shaped opening of a channel that is formed in the device layer and travels down to the level of the wafer occupied by the buried oxide layer, prior to manufacture of the device. The aspiration via is centered between the edge of the well and the center point of the well. The aspiration vias control the fluid transport and resulting pressure differential during device operation. FIG. 36C depicts a labeled cross section of a capture site featuring the aspiration via channel length 3601. Also, shown in the cross section are the locations of the device layer 3602, the buried oxide layer 3603 and the handle layer 3604 of the wafer from which the device was made. The verifiable acceptance criteria for the aspiration via channel length is 24 microns+/−0.5 microns. There are 4 aspiration via channels that are formed per well. The aspiration vias start at the bottom of the well and travel down completely through the device layer. The well depth impacts the aspiration via channel length. In this example, the aspiration via channel length was confirmed by SEM inspection of FIB cross sectioning capture sites throughout the device layer.

FIGS. 37A-37D show tilt-view SEM images of a capture site, focusing in on the projection (e.g., needle) 209. As shown in FIG. 37A, the projection length (e.g., needle length) 210 was measured by SEM from the projection base 3702 to the projection tip 3701, to be 3.5 microns. In some cases, the verifiable acceptance criteria is 3.5+/−0.5 microns. The projection is shown as being centered at the bottom of the well and projecting upward. The desired ultimate shape is a pointed pillar or conical needle. As shown in FIG. 37B, the projection base diameter (e.g., needle base diameter) 3703, defined to be 1 micron+0, −0.1 microns at the location shown. The projection base (e.g., needle base) refers to the point of the needle tip down to the point on the lower distal portion of the needle where there is no needle width greater than 1 micron. Ideally the projection base (e.g., needle base) is as close to the bottom of the well as possible. As shown in FIG. 37C the projection tip diameter was measured by SEM to be 150 nanometers. The verifiable acceptance criteria is for the projection tip diameter is 150+/−50 nanometers. The projection tip (e.g., needle tip) 3701 is defined as the top of the projection (e.g., needle, needle protrusion, or protrusion). The projection tip diameter (e.g., needle tip diameter) 3704 is measured from a point approximately 50 nanometers to 100 nanometers downward from the tip of the projection. As shown in FIG. 37D, the projection tip recession (e.g., needle tip recession) 3705 was measured by SEM to be 2.5 microns below the surface. The verifiable acceptance criteria is 2.5 microns to 3 microns. The projection tip recession can be greater than 2.5 microns as long as the projection length criteria is met. The projection tip recession is defined as the distance from the plane of the well top surface down to the tip of the needle. The plane of the top surface extends from the apex of one lobe of the clover leaf to the apex of the opposing lobe.

As shown in FIG. 38A, the overall chip size (e.g., device size) is defined by the device edge length by the device edge width, which for this example is 23.6 millimeters by 23.6 millimeters. The verifiable acceptance criteria is 23.6+/−0.5 millimeters for each of the four device edge lengths of the square device. As shown in FIG. 38B, the sub-array layout (e.g., array of sub-arrays) is composed of 10 rows and 10 columns of sub-arrays. The sub-array layout (e.g., array of sub-arrays) refers to the number of sub-array's aligned horizontally across the chip and the number of sub-arrays aligned vertically across the chip. As shown in FIG. 38C, the critical feature area of the sub-array layout is product of each critical feature edge length, which for this example is 15.53 millimeters by 15.53 millimeters. As shown in FIG. 38D, the border edge 3801 comprises a border edge width 3802 of 3.86 millimeters.

As shown in FIG. 39A, the gap between sub-arrays is 200 microns. The verifiable acceptance criteria is 200 microns+2, −0 microns. The gap between sub-arrays refers to the distance between the edge of a first sub-array to the edge of an adjacent, neighboring sub-array. As shown in FIG. 39B the sub-array size is the product of the edge lengths, which for this example is 1373 microns by 1373 microns. The verifiable acceptance criteria length is 1373 microns to 1383 microns. Where in in this example, the width is equal to the length. As shown in FIG. 39C the capture site layout, or equivalently, the well layout, is 108 capture site (e.g., wells) rows by 94 capture site (e.g., wells) columns.

As shown in FIG. 40A, the well center-to-center spacing for hexagonally packed (e.g., diamond packed) wells is 14 microns. The verifiable acceptance criteria for the well center-to-center spacing is 14+0/−0.5 microns. As shown in FIG. 40B, the well-to-well gap spacing refers to the distance between the well boundary perimeter of a first well to a well boundary perimeter of a second, neighboring well. The well-to-well gap spacing in this example is 2 microns. The verifiable acceptance criteria for the is 2+/−0.5 microns.

FIGS. 41A-41C show cross sectional diagrams of the wafer used to make the device. As shown in FIG. 41A, the device layer thickness of the device layer 3602 of the wafer used to fabricate the device described in this example was 30 microns thick. The verifiable acceptance criteria for the device layer thickness is 30+/−0.5 microns. The device layer thickness is defined as the distance from the device surface (e.g., top surface of the well) 202 to the top plane of the buried oxide layer 3603. The aspiration via channel that allows fluid transport and resulting pressure differential during device operation, runs through this device layer 3602. As shown in FIG. 41B, the buried oxide layer of the wafer is 2 microns thick. The verifiable acceptance criteria for the buried oxide layer thickness is 2+/−0.1 microns. The buried oxide layer thickness refers to the distance from top surface of the buried oxide layer to the bottom surface of the buried oxide layer. As shown in FIG. 41C, the handle layer 3604 of the wafer is 610 microns thick. The verifiable acceptance criteria for the handle layer thickness is 610+/−20 microns. The handle layer thickness refers to the distance from the bottom surface of the buried oxide layer to the backside surface of the wafer.

While preferred embodiments of the present disclosure have been shown and described herein, it will be obvious to those skilled in the art that such embodiments are provided by way of example only. Numerous variations, changes, and substitutions will now occur to those skilled in the rt without departing from the disclosure. It should be understood that various alternatives to the embodiments of the disclosure described herein may be employed in practicing the disclosure.

What is claimed is:

1. A system for single-cell mechanoporation comprising:
a) a static array of wells, wherein a well of the array of wells is configured to capture a single cell of a single cell suspension therein;
b) a static needle in the bottom surface of the well of the static array of wells configured to deliver or inject a cargo into the single cell captured in the well; and
c) one or more modules for generating a pressure gradient, wherein the pressure gradient is configured to induce a pressure-driven flow of the single cell suspension in the system, thereby leading the single cell to get captured in the well;
wherein capturing the single cell in the well is accomplished without centrifugation of the system or the static array.

2. The system of claim 1, wherein the single cell is captured in the well via sedimentation and flow focusing of the single cell in the well.

3. The system of claim 1, wherein the static needle is configured to deliver or inject the cargo into a section or an organelle of the single cell.

4. The system of claim 1, wherein the static needle is configured to deliver or inject the cargo into the nucleus of the single cell.

5. The system of claim 1, wherein the static needle is configured to deliver or inject the cargo into the single cell without back-and-forth movement.

6. The system of claim 1, wherein the length of the needle to the depth of the well comprises a ratio of about 30% to about 60%.

7. The system of claim 1, wherein the length of the needle to the depth of the well comprises a ratio of about 45% to about 65%.

8. The system of claim 1, wherein the well has a diameter of at most about 20 micrometers (microns).

9. The system of claim 1, wherein the well has a diameter of at most about 8 micrometers (microns).

10. The system of claim 1, wherein the static array of wells comprises at least 15,000 wells.

11. The system of claim 1, wherein the static array of wells comprises at least 200,000 wells.

12. The system of claim 1, wherein the system is capable of processing at least 1000 cells per cycle.

13. The system of claim 12, wherein the system is capable of processing at least about 100,000 cells per cycle.

14. The system of claim 13, wherein the system is capable of processing at least about 200,000 cells per cycle.

15. The system of claim 1, further comprising a computer software for controlling the performance of the system.

16. The system of claim 1, wherein the one or more modules for generating a pressure gradient comprise a reservoir containing the single cell suspension, a pressure controller, a pump, and a pump power supply.

17. The system of claim 16, further comprising a valve, a flow sensor, and a computer with control software.

18. The system of claim 16, wherein the pump is connected to the pressure controller, and wherein the pressure controller is pneumatically connected to the reservoir.

19. The system of claim 1, wherein the well comprises a depth of at most about 20 micrometers (microns).

20. The system of claim 1, wherein the static needle has a length of 1 micron to 8 microns.
